SEQUENCE LISTING (35 sequences)

SEQ ID NO: 1 - DNA, 4104 bp, Artificial Sequence
  Synthetic Polynucleotide
atggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgac

SEQ ID NO: 2 - DNA, 83 bp, Artificial Sequence
  Synthetic Polynucleotide
gttttagagctagaaatagcaagttaaaataaggctagtccgttatcaacttgaaaaagtggcaccgagtcggtgcttttttt

SEQ ID NO: 3 - DNA, 4104 bp, Artificial Sequence
  Synthetic Polynucleotide
atggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgaaagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgac

SEQ ID NO: 4 - DNA, 83 bp, Artificial Sequence; identical to SEQ ID NO: 2
  Synthetic Polynucleotide

SEQ ID NO: 5 - DNA, 4104 bp, Artificial Sequence
  Synthetic Polynucleotide
atggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgtgagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgcccgtgaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaagaatatcgttctacaaaagaagttttagatgccactcttatccatcaatccatcactggtctttatgaaacacgcattgatttgagtcagctaggaggtgac

SEQ ID NO: 6 - DNA, 83 bp, Artificial Sequence; identical to SEQ ID NO: 2
  Synthetic Polynucleotide

SEQ ID NO: 7 - DNA, 4104 bp, Artificial Sequence
  Synthetic Polynucleotide
atggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgtgagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacagtatcgttctacaaaagaagttttagatgccactcttatccatcaatccatcactggtctttatgaaacacgcattgatttgagtcagctaggaggtgac

SEQ ID NO: 8 - DNA, 83 bp, Artificial Sequence; identical to SEQ ID NO: 2
  Synthetic Polynucleotide

SEQ ID NO: 9 - DNA, 4104 bp, Artificial Sequence
  Synthetic Polynucleotide
atggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgaaagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacagtatcgttctacaaaagaagttttagatgccactcttatccatcaatccatcactggtctttatgaaacacgcattgatttgagtcagctaggaggtgac

SEQ ID NO: 10 - DNA, 83 bp, Artificial Sequence; identical to SEQ ID NO: 2
  Synthetic Polynucleotide

SEQ ID NO: 11 - DNA, 4104 bp, Artificial Sequence
  Synthetic Polynucleotide
atggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttgctgacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccagctcttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaagctcctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgac

SEQ ID NO: 12 - DNA, 83 bp, Artificial Sequence; identical to SEQ ID NO: 2
  Synthetic Polynucleotide

SEQ ID NO: 13 - DNA, 4104 bp, Artificial Sequence
  Synthetic Polynucleotide
atggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacagcttttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggagctttgtctcgaaaattgattaatggtattagggataagcaatctggcaaaacaatattagattttttgaaatcagatggttttgccaatcgcaattttatggctctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgcgctatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgac

SEQ ID NO: 14 - DNA, 83 bp, Artificial Sequence; identical to SEQ ID NO: 2
  Synthetic Polynucleotide

SEQ ID NO: 15 - DNA, 3159 bp, Artificial Sequence
  Synthetic Polynucleotide
atgaaacgtaattatatcttaggtctggcgattggtattactagtgttggctatggtattattgattacgagacgcgcgatgtcatcgacgctggggtgcgtctgtttaaggaagccaatgtggaaaacaatgagggtcgtcgtagtaaacgtggcgcccgtcgtctgaaacgccgtcgtcgtcatcgcattcagcgtgtgaaaaaactcctgtttgattataacctcctgaccgatcactcagaactctcgggcatcaacccgtatgaagcgcgtgttaaagggctgtctcagaaattatcagaggaagaattttcggccgcgctgctgcatttagccaaacgccgtggggtacacaacgtaaacgaagttgaagaagataccggaaacgaactgtccactaaagaacaaatctctcgcaactccaaagcactggaggaaaagtatgttgcggaattgcagctggagcgtttaaaaaaagacggtgaagtacgtggctccatcaatcgcttcaaaaccagcgattacgtgaaagaggcgaagcagctgctgaaagttcaaaaggcgtaccaccaactggatcagagctttattgacacctatatcgacctgctggaaacgcgccgtacttattatgaaggccctggggaaggatctccgttcggctggaaagacatcaaagaatggtatgaaatgctgatgggtcattgcacctatttcccggaggaacttcgtagtgttaagtacgcctataacgctgatctgtacaacgccctgaacgatttgaacaacctggtaattacccgcgatgagaacgaaaaactggagtattacgagaaattccagattatcgaaaatgttttcaaacaaaaaaagaaaccgacgcttaagcagattgcgaaagaaattttggttaacgaagaagatattaaagggtaccgtgttactagcacgggtaaaccggaattcactaacctgaaagtgtatcatgatatcaaagatattaccgcccgtaaggaaattattgaaaacgcggaattactggaccagatcgccaaaattctcaccatctatcaatcgtccgaggacatccaagaagagttgactaacctgaattcagagctgacacaggaagagatcgagcagattagcaacttaaaaggttataccggcactcacaatcttagtctgaaagctatcaacttgatccttgacgaactgtggcataccaacgataatcagatcgcgatctttaaccgtttaaaacttgtgccgaaaaaagtggatctgagccaacaaaaagagattccaaccaccttagtggatgattttattctcagcccggttgttaaacgtagctttattcaatcaatcaaagtgattaacgccattatcaaaaagtatgggctgccaaacgacattatcatcgaactggctcgtgaaaaaaattctaaggacgcgcaaaaaatgattaacgaaatgcagaaacgcaatcgccagacgaatgaacgcattgaagaaattatccgtactaccggaaaagaaaacgccaaatacctgatcgaaaagatcaaacttcacgacatgcaggaaggtaagtgtctgtactcccttgaagcaattcccctcgaggatttattgaacaatccgtttaattacgaggtggatcatattatcccacgctctgtgagctttgacaatagctttaacaataaagtcttggtgaaacaagaagaagcttctaaaaaagggaaccgcacccctttccagtacctgagcagttcggatagtaagatctcatatgaaactttcaaaaaacatattctgaacttggcaaagggcaaaggccgtatctcaaaaacaaagaaagaatatcttttggaagagcgtgatatcaatcgcttctcagttcaaaaagacttcatcaaccgcaacttagtggacacccgttatgccacacgtggcttaatgaacttactgcgcagctactttcgcgtgaacaatcttgacgtgaaggtgaaaagcatcaacggcggcttcacatctttcttgcgtcgtaaatggaagtttaaaaaagagcgcaataagggttacaagcatcacgccgaagatgcgctgatcatcgcgaacgcggattttatcttcaaggaatggaaaaaattggataaagccaaaaaagtgatggaaaaccaaatgttcgaagagaagcaggcagaaagtatgccggaaatcgaaacggaacaagagtacaaagaaatcttcattacgccacatcaaattaagcacattaaagatttcaaagactataaatactcacatcgtgttgacaaaaaaccgaatcgtgaattgattaacgatacgttatactcgacgcgcaaggatgataaagggaacacattaatcgtcaataatttgaacgggctgtacgataaagacaacgataaactcaaaaagctgatcaataagagtccggagaaactgttaatgtatcatcatgacccccagacttatcagaagctgaaattaatcatggaacaatacggggacgaaaaaaacccactttataaatattatgaggaaacaggcaactatctgacaaagtactctaaaaaggataatggtccggtcatcaaaaaaattaaatactatggtaacaaactcaatgcgcatctcgacatcaccgatgattacccaaattcccgtaacaaagtggttaaactttctctgaaaccttatcgctttgatgtatatctggacaatggcgtttacaaatttgttaccgttaaaaacttagatgttattaaaaaagaaaattactacgaagtcaactcaaaatgttacgaagaggcgaaaaaactgaaaaaaatctccaaccaagcggaattcattgcgtccttttataataatgacttaatcaagattaacggtgagttgtaccgcgtcattggagtgaataatgatctgctgaaccgtatcgaagtgaacatgatcgatattacttatcgcgaatatctggaaaatatgaacgacaaacgtccacctcgtatcattaaaaccatcgctagtaaaacccaaagcatcaaaaaatattccacggatattttaggtaatctctatgaagttaaatcgaagaaacatccccagatcattaaaaaaggc

SEQ ID NO: 16 - DNA, 83 bp, Artificial Sequence
  Synthetic Polynucleotide
gttttagtactctggaaacagaatctactaaaacaaggcaaaatgccgtgtttatctcgtcaacttgttggcgagattttttt

SEQ ID NO: 17 - DNA, 3159 bp, Artificial Sequence
  Synthetic Polynucleotide
atgaaacgtaattatatcttaggtctggcgattggtattactagtgttggctatggtattattgattacgagacgcgcgatgtcatcgacgctggggtgcgtctgtttaaggaagccaatgtggaaaacaatgagggtcgtcgtagtaaacgtggcgcccgtcgtctgaaacgccgtcgtcgtcatcgcattcagcgtgtgaaaaaactcctgtttgattataacctcctgaccgatcactcagaactctcgggcatcaacccgtatgaagcgcgtgttaaagggctgtctcagaaattatcagaggaagaattttcggccgcgctgctgcatttagccaaacgccgtggggtacacaacgtaaacgaagttgaagaagataccggaaacgaactgtccactaaagaacaaatctctcgcaactccaaagcactggaggaaaagtatgttgcggaattgcagctggagcgtttaaaaaaagacggtgaagtacgtggctccatcaatcgcttcaaaaccagcgattacgtgaaagaggcgaagcagctgctgaaagttcaaaaggcgtaccaccaactggatcagagctttattgacacctatatcgacctgctggaaacgcgccgtacttattatgaaggccctggggaaggatctccgttcggctggaaagacatcaaagaatggtatgaaatgctgatgggtcattgcacctatttcccggaggaacttcgtagtgttaagtacgcctataacgctgatctgtacaacgccctgaacgatttgaacaacctggtaattacccgcgatgagaacgaaaaactggagtattacgagaaattccagattatcgaaaatgttttcaaacaaaaaaagaaaccgacgcttaagcagattgcgaaagaaattttggttaacgaagaagatattaaagggtaccgtgttactagcacgggtaaaccggaattcactaacctgaaagtgtatcatgatatcaaagatattaccgcccgtaaggaaattattgaaaacgcggaattactggaccagatcgccaaaattctcaccatctatcaatcgtccgaggacatccaagaagagttgactaacctgaattcagagctgacacaggaagagatcgagcagattagcaacttaaaaggttataccggcactcacaatcttagtctgaaagctatcaacttgatccttgacgaactgtggcataccaacgataatcagatcgcgatctttaaccgtttaaaacttgtgccgaaaaaagtggatctgagccaacaaaaagagattccaaccaccttagtggatgattttattctcagcccggttgttaaacgtagctttattcaatcaatcaaagtgattaacgccattatcaaaaagtatgggctgccaaacgacattatcatcgaactggctcgtgaaaaaaattctaaggacgcgcaaaaaatgattaacgaaatgcagaaacgcaatgctgcgacgaatgaacgcattgaagaaattatccgtactaccggaaaagaaaacgccaaatacctgatcgaaaagatcaaacttcacgacatgcaggaaggtaagtgtctgtactcccttgaagcaattcccctcgaggatttattgaacaatccgtttaattacgaggtggatcatattatcccacgctctgtgagctttgacaatagctttaacaataaagtcttggtgaaacaagaagaagcttctaaaaaagggaaccgcacccctttccagtacctgagcagttcggatagtaagatctcatatgaaactttcaaaaaacatattctgaacttggcaaagggcaaaggccgtatctcaaaaacaaagaaagaatatcttttggaagagcgtgatatcaatcgcttctcagttcaaaaagacttcatcaaccgcaacttagtggacacccgttatgccacagctgcgttaatgaacttactgcgcagctactttcgcgtgaacaatcttgacgtgaaggtgaaaagcatcaacggcggcttcacatctttcttgcgtcgtaaatggaagtttaaaaaagagcgcaataagggttacaagcatcacgccgaagatgcgctgatcatcgcgaacgcggattttatcttcaaggaatggaaaaaattggataaagccaaaaaagtgatggaaaaccaaatgttcgaagagaagcaggcagaaagtatgccggaaatcgaaacggaacaagagtacaaagaaatcttcattacgccacatcaaattaagcacattaaagatttcaaagactataaatactcacatcgtgttgacaaaaaaccgaatcgtgaattgattaacgatacgttatactcgacgcgcaaggatgataaagggaacacattaatcgtcaataatttgaacgggctgtacgataaagacaacgataaactcaaaaagctgatcaataagagtccggagaaactgttaatgtatcatcatgacccccagacttatcagaagctgaaattaatcatggaacaatacggggacgaaaaaaacccactttataaatattatgaggaaacaggcaactatctgacaaagtactctaaaaaggataatggtccggtcatcaaaaaaattaaatactatggtaacaaactcaatgcgcatctcgacatcaccgatgattacccaaattcccgtaacaaagtggttaaactttctctgaaaccttatcgctttgatgtatatctggacaatggcgtttacaaatttgttaccgttaaaaacttagatgttattaaaaaagaaaattactacgaagtcaactcaaaatgttacgaagaggcgaaaaaactgaaaaaaatctccaaccaagcggaattcattgcgtccttttataataatgacttaatcaagattaacggtgagttgtaccgcgtcattggagtgaataatgatctgctgaaccgtatcgaagtgaacatgatcgatattacttatcgcgaatatctggaaaatatgaacgacaaacgtccacctcgtatcattaaaaccatcgctagtaaaacccaaagcatcaaaaaatattccacggatattttaggtaatctctatgaagttaaatcgaagaaacatccccagatcattaaaaaaggc

SEQ ID NO: 18 - DNA, 83 bp, Artificial Sequence; identical to SEQ ID NO: 16
  Synthetic Polynucleotide

SEQ ID NO: 19 - DNA, 3363 bp, Artificial Sequence
  Synthetic Polynucleotide
atgagtgaccttgtgttgggcctggccatcggcattggttcggtcggagttggaattctgaacaaggtcaccggggaaattattcataaaaattcacgtattttcccggcggcacaagcggaaaacaacttagtccgccgcactaaccgccagggccgccgtctggcgcgtcgtaaaaaacatcgccgcgtccgtctgaatcgcttgtttgaggagagcggtttgattactgactttacgaaaatctccattaatctcaacccgtatcagctccgtgtgaagggcctcacggacgaactgagcaacgaggaattattcatcgcactgaaaaacatggtgaaacatcgcggtattagttatctggacgatgccagcgacgatggcaacagtagcgttggtgactatgctcaaatcgtgaaagagaacagtaaacaactggaaactaaaaccccgggtcaaatccagttagaacgttatcaaacgtatggtcagctgcgtggcgatttcacagtggaaaaggatggtaagaagcatcgtctgattaatgtgttccctacctcggcatatcgttccgaggccttgcgtatcctgcagactcagcaagaatttaatccgcagatcaccgatgagttcatcaaccgctaccttgaaattcttaccggcaaacgcaagtattatcacggtccgggtaatgaaaaaagccgcacggactacggtcgttatcgcacatcaggcgaaactttggacaacatttttggcattttaattggtaaatgtacgttctacccggatgagttccgcgcggcgaaagcgtcctatacagcgcaagagtttaatctgttaaatgatttgaataatctgacagtaccgactgagacgaaaaaactttctaaagaacagaaaaatcagatcattaattacgtaaagaacgagaaagcgatgggcccggcgaaactgtttaaatacattgccaaacttctgtcatgcgatgtggcagatattaaaggctaccgcattgataaaagcggcaaagctgaaattcatacgtttgaggcctatcgcaaaatgaaaactttagaaacgcttgacattgaacagatggatcgcgaaacgctggataaattggcctacgtactcactctgaatacggaacgcgaaggcatccaggaagcgcttgagcatgagtttgcagatggctcgttttcccaaaaacaagttgacgaattggtccaattccgcaaagccaattcctccatttttggtaaaggttggcataactttagtgtgaagttaatgatggagttgatccccgaactctacgaaaccagtgaggaacaaatgaccatcttgacgcgcctgggtaaacagaaaacgacgagctcgagcaacaaaacgaaatatattgatgagaagctgttgacggaggagatttataacccggttgtggcaaagagcgtccgccaggccatcaaaatcgtcaacgctgccattaaagaatacggcgattttgataacatcgttattgaaatggcgcgcgagactaatgaggatgatgaaaaaaaagcgattcagaaaatccaaaaagccaacaaagatgaaaaggatgcggccatgctgaaagccgcaaaccaatacaatggtaaggccgagcttccgcattctgtgtttcatgggcataagcaattagcgacgaaaatccgtctgtggcatcagcagggcgaacgttgcctttacaccggtaaaacaatctcaattcatgatttaattaacaatagtaaccagtttgaggtcgatgcgattctgccgctgagtattacttttgatgactcccttgctaacaaagtcttggtttatgcgactgccaaccaggagaaaggtcagcgcaccccctatcaggcgctggattcgatggacgatgcatggtcatttcgtgaactgaaagcgttcgtgcgtgaaagtaaaacactgtcgaataaaaaaaaagaatatctgttgaccgaagaggatatttccaaatttgatgtccgcaaaaaatttattgaacgtaacctggtagatactcgctacgccagtcgcgttgtcctgaacgccctgcaagaacatttccgtgctcataagattgacactaaagtgtctgtagtgcgtgggcagtttacttcccagttacgccgccattgggggatcgaaaaaacccgcgatacctatcaccatcacgcggtcgacgcgttgattatcgctgcaagttcgcagctcaacctgtggaaaaaacagaaaaacacgctggtttcgtattcggaagatcagttgctggatattgaaactggtgagctgattagtgacgatgaatacaaagaaagcgtatttaaggcaccataccagcattttgtcgataccctgaagtccaaagaatttgaagattcaatcctcttttcctatcaggtggacagtaaatttaaccgtaaaattagtgatgcaacaatttatgcgacgcgtcaagctaaggtgggcaaagataaagcggacgaaacctacgtcctcggtaagatcaaagatatctatactcaggatggctatgatgccttcatgaaaatctacaaaaaagataaatctaagtttttgatgtatcgtcatgacccgcaaaccttcgaaaaagtcattgagccgatcttggagaactatccgaacaaacaaatcaacgaaaaaggcaaagaagttccgtgcaatcccttcttaaaatataaagaagaacatggttatatccgcaagtatagcaagaagggtaacgggccggaaattaagtctctgaaatactatgactcgaagttgggcaatcatattgacatcaccccgaaagattcaaataataaagttgtgctgcagagcgtaagtccgtggcgtgcggatgtctatttcaacaaaacaacaggaaaatatgaaatcctgggcctcaaatatgccgacttacaattcgaaaagggtaccggtacatacaaaatctcccaggaaaaatacaacgatattaaaaaaaaagaaggcgtagatagcgactctgagttcaaatttacgttatataagaacgatctgctgttagtaaaagataccgaaaccaaggaacagcagttatttcgtttcctgtcgcgtaccatgcccaaacagaaacactatgtggaacttaaaccatacgacaaacaaaaattcgagggtggtgaagcgctcattaaagttttaggtaatgtggcaaattccggtcagtgcaaaaaaggtttaggtaaaagtaatatttccatttataaagttcgcaccgatgtactggggaatcagcatattattaaaaatgaaggagacaagcccaaactggacttt

SEQ ID NO: 20 - DNA, 132 bp, Artificial Sequence
  Synthetic Polynucleotide
gtttttgtactctcaagatttaagtaactgtacaacgaaacttacacagttacttaaatcttgcagaagctacaaagataaggcttcatgccgaaatcaacaccctgtcattttatggcagggtgttttttt

SEQ ID NO: 21 - DNA, 3900 bp, Artificial Sequence
  Synthetic Polynucleotide
atgagcatctaccaggagttcgtcaacaagtattcactgagtaagacactgcggttcgagctgatcccacagggcaagacactggagaacatcaaggcccgaggcctgattctggacgatgagaagcgggcaaaagactataagaaagccaagcagatcattgataaataccaccagttctttatcgaggaaattctgagctccgtgtgcatcagtgaggatctgctgcagaattactcagacgtgtacttcaagctgaagaagagcgacgatgacaacctgcagaaggacttcaagtccgccaaggacaccatcaagaaacagattagcgagtacatcaaggactccgaaaagtttaaaaatctgttcaaccagaatctgatcgatgctaagaaaggccaggagtccgacctgatcctgtggctgaaacagtctaaggacaatgggattgaactgttcaaggctaactccgatatcactgatattgacgaggcactggaaatcatcaagagcttcaagggatggaccacatactttaaaggcttccacgagaaccgcaagaacgtgtactccagcaacgacattcctacctccatcatctaccgaatcgtcgatgacaatctgccaaagttcctggagaacaaggccaaatatgaatctctgaaggacaaagctcccgaggcaattaattacgaacagatcaagaaagatctggctgaggaactgacattcgatatcgactataagactagcgaggtgaaccagagggtcttttccctggacgaggtgtttgaaatcgccaatttcaacaattacctgaaccagtccggcattactaaattcaataccatcattggcgggaagtttgtgaacggggagaataccaagcgcaagggaattaacgaatacatcaatctgtatagccagcagatcaacgacaaaactctgaagaaatacaagatgtctgtgctgttcaaacagatcctgagtgataccgagtccaagtcttttgtcattgataaactggaagatgactcagacgtggtcactaccatgcagagcttttatgagcagatcgccgctttcaagacagtggaggaaaaatctattaaggaaactctgagtctgctgttcgatgacctgaaagcccagaagctggacctgagtaagatctacttcaaaaacgataagagtctgacagacctgtcacagcaggtgtttgatgactattccgtgattgggaccgccgtcctggagtacattacacagcagatcgctccaaagaacctggataatccctctaagaaagagcaggaactgatcgctaagaaaaccgagaaggcaaaatatctgagtctggaaacaattaagctggcactggaggagttcaacaagcacagggatattgacaaacagtgccgctttgaggaaatcctggccaacttcgcagccatccccatgatttttgatgagatcgcccagaacaaagacaatctggctcagatcagtattaagtaccagaaccagggcaagaaagacctgctgcaggcttcagcagaagatgacgtgaaagccatcaaggatctgctggaccagaccaacaatctgctgcacaagctgaaaatcttccatattagtcagtcagaggataaggctaatatcctggataaagacgaacacttctacctggtgttcgaggaatgttacttcgagctggcaaacattgtccccctgtataacaagattaggaactacatcacacagaagccttactctgacgagaagtttaaactgaacttcgaaaatagtaccctggccaacgggtgggataagaacaaggagcctgacaacacagctatcctgttcatcaaggatgacaagtactatctgggagtgatgaataagaaaaacaataagatcttcgatgacaaagccattaaggagaacaaaggggaaggatacaagaaaatcgtgtataagctgctgcccggcgcaaataagatgctgcctaaggtgttcttcagcgccaagagtatcaaattctacaacccatccgaggacatcctgcggattagaaatcactcaacacatactaagaacgggagcccccagaagggatatgagaaatttgagttcaacatcgaggattgcaggaagtttattgacttctacaagcagagcatctccaaacaccctgaatggaaggattttggcttccggttttccgacacacagagatataactctatcgacgagttctaccgcgaggtggaaaatcaggggtataagctgacttttgagaacatttctgaaagttacatcgacagcgtggtcaatcagggaaagctgtacctgttccagatctataacaaagatttttcagcatacagcaagggcagaccaaacctgcatacactgtactggaaggccctgttcgatgagaggaatctgcaggacgtggtctataaactgaacggagaggccgaactgttttaccggaagcagtctattcctaagaaaatcactcacccagctaaggaggccatcgctaacaagaacaaggacaatcctaagaaagagagcgtgttcgaatacgatctgattaaggacaagcggttcaccgaagataagttctttttccattgtccaatcaccattaacttcaagtcaagcggcgctaacaagttcaacgacgagatcaatctgctgctgaaggaaaaagcaaacgatgtgcacatcctgagcattgctcgaggagagcggcatctggcctactataccctggtggatggcaaagggaatatcattaagcaggatacattcaacatcattggcaatgaccggatgaaaaccaactaccacgataaactggctgcaatcgagaaggatagagactcagctaggaaggactggaagaaaatcaacaacattaaggagatgaaggaaggctatctgagccaggtggtccatgagattgcaaagctggtcatcgaatacaatgccattgtggtgttcgaggatctgaacttcggctttaagagggggcgctttaaggtggaaaaacaggtctatcagaagctggagaaaatgctgatcgaaaagctgaattacctggtgtttaaagataacgagttcgacaagaccggaggcgtcctgagagcctaccagctgacagctccctttgaaactttcaagaaaatgggaaaacagacaggcatcatctactatgtgccagccggattcacttccaagatctgccccgtgaccggctttgtcaaccagctgtaccctaaatatgagtcagtgagcaagtcccaggaatttttcagcaagttcgataagatctgttataatctggacaaggggtacttcgagttttccttcgattacaagaacttcggcgacaaggccgctaaggggaaatggaccattgcctccttcggatctcgcctgatcaactttcgaaattccgataaaaaccacaattgggacactagggaggtgtacccaaccaaggagctggaaaagctgctgaaagactactctatcgagtatggacatggcgaatgcatcaaggcagccatctgtggcgagagtgataagaaatttttcgccaagctgacctcagtgctgaatacaatcctgcagatgcggaactcaaagaccgggacagaactggactatctgattagccccgtggctgatgtcaacggaaacttcttcgacagcagacaggcacccaaaaatatgcctcaggatgcagacgccaacggggcctaccacatcgggctgaagggactgatgctgctgggccggatcaagaacaatcaggaggggaagaagctgaacctggtcattaagaacgaggaatacttcgagtttgtccagaatagaaataac

SEQ ID NO: 22 - DNA, 20 bp, Artificial Sequence
  Synthetic Polynucleotide
taatttctactgttgtagat

SEQ ID NO: 23 - DNA, 3921 bp, Artificial Sequence
  Synthetic Polynucleotide
atgacccaatttgaagggtttaccaacctttaccaagttagcaaaaccctgcgtttcgagttgatcccacaaggaaaaaccctgaaacacattcaggaacagggattcattgaagaggataaagctcgtaacgatcactataaggaacttaagccgattatcgatcgcatttacaaaacgtatgcggatcagtgcctgcagcttgtgcaactggactgggaaaacctgagcgccgccattgattcttatcgcaaagaaaagaccgaagaaacccgtaacgccttgattgaggaacaagccacgtatcgcaatgccattcatgattatttcatcggccgtacggacaatttaacggacgcaattaacaaacgccacgctgagatctacaagggcctgtttaaagctgaactgtttaatggcaaagtgctcaagcaactgggcaccgtcaccaccaccgaacatgagaacgctctcctgcgcagttttgataaattcaccacatatttttcggggttctatgaaaaccgcaaaaatgttttctcagcggaagatattagcaccgcgatccctcatcgtatcgtgcaggataacttccctaagttcaaagagaactgtcacatctttactcgcctgattaccgctgtaccctcgttgcgcgaacactttgaaaacgtcaaaaaagcaatcggcatttttgtatcgaccagtattgaagaggttttcagcttcccgttctataaccagctgctgacgcaaacgcaaatcgatctgtacaatcagctgttaggtggcatttcccgcgaagcgggcaccgaaaaaatcaaagggctgaatgaagtactgaacctggcgatccaaaaaaacgacgaaacggcgcacattatcgcctctctgccgcatcgctttattcccctgttcaagcagatcctgagcgatcgcaacactctgagtttcattctcgaagaattcaaatctgacgaagaagttattcagtctttctgtaaatataaaaccctgctgcgtaacgagaatgtactggaaaccgcggaagcactgtttaacgagctgaattccattgatctgacccatatttttatctctcacaaaaaactggaaaccattagctctgcgctctgcgatcattgggacacgcttcgcaacgcgctctacgagcgccgtatttccgagttgacggggaaaatcaccaaatccgctaaggaaaaagttcagcgtagcttgaaacatgaagatattaatctgcaggagatcattagtgcggctggtaaagaactttcagaagcgtttaaacagaaaacttcggaaatcttatcgcacgcacatgcggctcttgatcagccgcttccaacgaccctgaaaaaacaggaggaaaaagaaattctgaaaagtcaactggatagtttattgggcctgtaccacctgcttgactggtttgcggtggatgaatccaatgaagtcgatcccgagttttccgcacgcctgactgggatcaaactggaaatggaaccaagcctgtctttttacaacaaagcgcgtaactacgcgaccaaaaagccatacagtgttgaaaagtttaaattgaactttcagatgccaaccctggcgagtggctgggatgtgaataaagaaaaaaataatggtgcaatcctgtttgttaaaaatggactgtactatctgggtattatgccgaaacaaaaaggtcgctataaagcgctgtcatttgagccgacagaaaagacgagtgaaggctttgataaaatgtactatgattactttccagatgcagcgaaaatgattcctaagtgtagtacccagctgaaggcagtaacagcccacttccagacccacaccaccccgattttgttaagcaacaactttattgaaccgctggaaattacaaaagaaatctatgaccttaacaacccagagaaggaacccaagaagttccaaacggcgtatgcgaaaaagactggcgatcaaaaagggtatcgtgaggcgctgtgcaaatggattgattttacgcgcgattttctgagcaaatatacaaaaaccacctcgattgatctgagtagcctgcgtcctagtagtcagtataaggatttgggcgaatactatgcggaacttaatccgctgctgtaccatatttcgttccagcgtatcgcggaaaaagagattatggatgcggtagaaaccgggaaattgtatctgttccagatttacaataaagatttcgccaagggccatcacggtaagcccaacctgcatactttatattggacaggtttattcagcccagaaaatttagcgaaaacaagcatcaaactgaacggccaggctgaactgttctatcgcccgaaaagtcgcatgaaacgcatggcacatcgtttaggtgaaaagatgttgaacaaaaaacttaaagatcagaaaactccgattcctgacactctctaccaagaactgtacgattacgtcaaccatcgtctgagccatgatctgagtgatgaagcacgcgctctgctgccgaacgtgattaccaaggaagtcagtcacgaaattattaaagaccgccgcttcacatccgataaattcttcttccacgtgccaattacgcttaattaccaggcagcaaactctccttccaaatttaatcagcgcgttaatgcgtatctgaaagagcacccggaaacaccgatcattggtatcgcccgcggggaacgtaatctcatctatattactgttattgattcgaccggcaaaattctggagcagcgctctcttaatacgatccagcagtttgactatcaaaaaaagctggacaaccgcgagaaggaacgcgtcgcggcccgccaagcctggagcgttgttggcacaatcaaagatctgaagcagggttaccttagtcaagttatccacgaaattgttgatcttatgattcattatcaggccgttgtggtgctggaaaaccttaactttggctttaaaagcaaacgcacggggatcgctgaaaaagcggtataccagcagttcgaaaaaatgctgattgataaactcaattgcctcgtgctgaaagattacccggctgaaaaagtgggtggtgtgctgaatccatatcaactgacagatcaattcacctcttttgctaaaatgggcactcaaagcggttttttgttttacgtcccggcgccctatactagcaagatcgacccgctgacaggttttgtggatccgttcgtttggaaaaccatcaaaaatcatgaatcgcgtaaacactttctggagggctttgatttcttgcactatgatgtgaaaaccggcgattttatcctgcattttaagatgaaccgtaatttgagcttccagcgcggtctgccaggtttcatgcccgcgtgggacattgttttcgaaaagaatgagacacagttcgacgccaaaggaaccccgtttatcgcgggtaaacgcattgttccggtcatcgagaatcatcgttttaccggtcgttaccgtgatttgtatcctgcaaacgaactcatcgcattgttggaagaaaagggaatcgtgtttcgtgatggttcgaatatcctgcctaaactgctggagaacgatgactcacacgctattgataccatggtcgctctgatccgctcggtcttacagatgcgtaacagcaacgcggctacgggcgaggattacattaactcgccggttcgcgatctgaacggcgtctgctttgactcgcgctttcaaaatccagaatggccgatggatgcggacgcgaatggtgcgtaccacatcgctcttaaagggcagttactgttaaaccatttgaaagaatctaaagacctgaagctgcaaaacggtatcagcaaccaggattggctcgcgtatatccaagaactgcgtaac

SEQ ID NO: 24 - DNA, 20 bp, Artificial Sequence
  Synthetic Polynucleotide
taatttctactcttgtagat

SEQ ID NO: 25 - DNA, 3684 bp, Artificial Sequence
  Synthetic Polynucleotide
atgagcaagctggagaagtttacaaactgctactccctgtctaagaccctgaggttcaaggccatccctgtgggcaagacccaggagaacatcgacaataagcggctgctggtggaggacgagaagagagccgaggattataagggcgtgaagaagctgctggatcgctactatctgtcttttatcaacgacgtgctgcacagcatcaagctgaagaatctgaacaattacatcagcctgttccggaagaaaaccagaaccgagaaggagaataaggagctggagaacctggagatcaatctgcggaaggagatcgccaaggccttcaagggcaacgagggctacaagtccctgtttaagaaggatatcatcgagacaatcctgccagagttcctggacgataaggacgagatcgccctggtgaacagcttcaatggctttaccacagccttcaccggcttctttgataacagagagaatatgttttccgaggaggccaagagcacatccatcgccttcaggtgtatcaacgagaatctgacccgctacatctctaatatggacatcttcgagaaggtggacgccatctttgataagcacgaggtgcaggagatcaaggagaagatcctgaacagcgactatgatgtggaggatttctttgagggcgagttctttaactttgtgctgacacaggagggcatcgacgtgtataacgccatcatcggcggcttcgtgaccgagagcggcgagaagatcaagggcctgaacgagtacatcaacctgtataatcagaaaaccaagcagaagctgcctaagtttaagccactgtataagcaggtgctgagcgatcgggagtctctgagcttctacggcgagggctatacatccgatgaggaggtgctggaggtgtttagaaacaccctgaacaagaacagcgagatcttcagctccatcaagaagctggagaagctgttcaagaattttgacgagtactctagcgccggcatctttgtgaagaacggccccgccatcagcacaatctccaaggatatcttcggcgagtggaacgtgatccgggacaagtggaatgccgagtatgacgatatccacctgaagaagaaggccgtggtgaccgagaagtacgaggacgatcggagaaagtccttcaagaagatcggctccttttctctggagcagctgcaggagtacgccgacgccgatctgtctgtggtggagaagctgaaggagatcatcatccagaaggtggatgagatctacaaggtgtatggctcctctgagaagctgttcgacgccgattttgtgctggagaagagcctgaagaagaacgacgccgtggtggccatcatgaaggacctgctggattctgtgaagagcttcgagaattacatcaaggccttctttggcgagggcaaggagacaaacagggacgagtccttctatggcgattttgtgctggcctacgacatcctgctgaaggtggaccacatctacgatgccatccgcaattatgtgacccagaagccctactctaaggataagttcaagctgtattttcagaaccctcagttcatgggcggctgggacaaggataaggagacagactatcgggccaccatcctgagatacggctccaagtactatctggccatcatggataagaagtacgccaagtgcctgcagaagatcgacaaggacgatgtgaacggcaattacgagaagatcaactataagctgctgcccggccctaataagatgctgccaaaggtgttcttttctaagaagtggatggcctactataaccccagcgaggacatccagaagatctacaagaatggcacattcaagaagggcgatatgtttaacctgaatgactgtcacaagctgatcgacttctttaaggatagcatctcccggtatccaaagtggtccaatgcctacgatttcaacttttctgagacagagaagtataaggacatcgccggcttttacagagaggtggaggagcagggctataaggtgagcttcgagtctgccagcaagaaggaggtggataagctggtggaggagggcaagctgtatatgttccagatctataacaaggacttttccgataagtctcacggcacacccaatctgcacaccatgtacttcaagctgctgtttgacgagaacaatcacggacagatcaggctgagcggaggagcagagctgttcatgaggcgcgcctccctgaagaaggaggagctggtggtgcacccagccaactcccctatcgccaacaagaatccagataatcccaagaaaaccacaaccctgtcctacgacgtgtataaggataagaggttttctgaggaccagtacgagctgcacatcccaatcgccatcaataagtgccccaagaacatcttcaagatcaatacagaggtgcgcgtgctgctgaagcacgacgataacccctatgtgatcggcatcgctaggggcgagcgcaatctgctgtatatcgtggtggtggacggcaagggcaacatcgtggagcagtattccctgaacgagatcatcaacaacttcaacggcatcaggatcaagacagattaccactctctgctggacaagaaggagaaggagaggttcgaggcccgccagaactggacctccatcgagaatatcaaggagctgaaggccggctatatctctcaggtggtgcacaagatctgcgagctggtggagaagtacgatgccgtgatcgccctggaggacctgaactctggctttaagaatagccgcgtgaaggtggagaagcaggtgtatcagaagttcgagaagatgctgatcgataagctgaactacatggtggacaagaagtctaatccttgtgcaacaggcggcgccctgaagggctatcagatcaccaataagttcgagagctttaagtccatgtctacccagaacggcttcatcttttacatccctgcctggctgacatccaagatcgatccatctaccggctttgtgaacctgctgaaaaccaagtataccagcatcgccgattccaagaagttcatcagctcctttgacaggatcatgtacgtgcccgaggaggatctgttcgagtttgccctggactataagaacttctctcgcacagacgccgattacatcaagaagtggaagctgtactcctacggcaaccggatcagaatcttccggaatcctaagaagaacaacgtgttcgactgggaggaggtgtgcctgaccagcgcctataaggagctgttcaacaagtacggcatcaattatcagcagggcgatatcagagccctgctgtgcgagcagtccgacaaggccttctactctagctttatggccctgatgagcctgatgctgcagatgcggaacagcatcacaggccgcaccgacgtggattttctgatcagccctgtgaagaactccgacggcatcttctacgatagccggaactatgaggcccaggagaatgccatcctgccaaagaacgccgacgccaatggcgcctataacatcgccagaaaggtgctgtgggccatcggccagttcaagaaggccgaggacgagaagctggataaggtgaagatcgccatctctaacaaggagtggctggagtacgcccagaccagcgtgaagcac

SEQ ID NO: 26 - DNA, 21 bp, Artificial Sequence
  Synthetic Polynucleotide
taatttctactaagtgtagat

SEQ ID NO: 27 - DNA, 18 bp, Artificial Sequence
  Synthetic Polynucleotide
taatacgactcactatag

SEQ ID NO: 28 - DNA, 143 bp, Artificial Sequence
  Synthetic Polynucleotide
gcttcctcgctcactgactcgctgcacgaggcagacctcagcgctagcggagtgtatactggcttactatgttggcactgatgagggtgtcagtgaagtgcttcatgtggcaggagaaaaaaggctgcaccggtgcgtcagca

SEQ ID NO: 29 - DNA, 33 bp, Artificial Sequence
  Synthetic Polynucleotide
aacgatcgttggctgtgttgacaattaatcatc

SEQ ID NO: 30 - DNA, 33 bp, Artificial Sequence
  Synthetic Polynucleotide
tactccaccgttggcttttttccctatcagtga

SEQ ID NO: 31 - DNA, 33 bp, Artificial Sequence
  Synthetic Polynucleotide
acttttcatactcccgccattcagagaagaaac

SEQ ID NO: 32 - RNA, 20 nt, Artificial Sequence
  Synthetic Polynucleotide
uaauuucuacucuuguagau

SEQ ID NO: 33 - RNA, 83 nt, Artificial Sequence
  Synthetic Polynucleotide
guuuuagagcuagaaauagcaaguuaaaauaaggcuaguccguuaucaacuugaaaaaguggcaccgagucggugcuuuuuuu

SEQ ID NO: 34 - RNA, 83 nt, Artificial Sequence
  Synthetic Polynucleotide
guuuuaguacucuggaaacagaaucuacuaaaacaaggcaaaaugccguguuuaucucgucaacuuguuggcgagauuuuuuu

SEQ ID NO: 35 - RNA, 130 nt, Artificial Sequence
  Synthetic Polynucleotide
guuuuuguacucucaagauuuaaguaacuguacaacgaaacuuacacaguuacuuaaaucuugcagaagcuacaaagauaaggcuucaugccgaaaucaacacccugucauuuuauggcaggguguuuuu

(19) United States
(12) Patent Application Publication
(10) Pub. No.: US 2019/0345501 A1
(43) Pub. Date: Nov. 14, 2019

(54) METHODS AND COMPOSITIONS FOR RNA-GUIDED GENETIC CIRCUITS

(71) Applicant: Massachusetts Institute of Technology, Cambridge, MA (US)

(72) Inventors: Alec Andrew Nielsen, Cambridge, MA (US); Christopher A. Voigt, Belmont, MA (US)

(73) Assignee: Massachusetts Institute of Technology, Cambridge, MA (US)

(21) Appl. No.: 16/080,707

(22) PCT Filed: Feb. 7, 2018
(86) PCT No.: PCT/US2018/017169

§ 371 Date: Aug. 29, 2018

Related U.S. Application Data
(60) Provisional application No. 62/455,719, filed on Feb. 7, 2017.

(51) Int. Cl. C12N 15/63; C12N 15/11; C12N 9/22; C12N 15/70

(52) U.S. Cl. CPC C12N 15/635; C12N 15/11; C12N 2800/80; C12N 15/70; C12N 2310/20; C12N 9/22

(57) ABSTRACT

Some aspects provide genetic circuits to engineer complex and adaptive cellular behaviors. Methods of controlling expression of output sequence(s) are also provided herein using genetic circuits that employ catalytically inactive endonucleases.

### RELATED APPLICATIONS

This application is a national stage filing under 35 U.S.C. § 371 of International Patent Application Serial No. PCT/US2018/017169, filed Feb. 7, 2018, entitled “METHODS AND COMPOSITIONS FOR RNA-GUIDED GENETIC CIRCUITS,” which claims the benefit under 35 U.S.C. § 119(e) of U.S. Provisional Application No. 62/455,719, filed Feb. 7, 2017, entitled “METHODS AND COMPOSITIONS FOR RNA-GUIDED GENETIC CIRCUITS,” the contents of each of which is incorporated by reference herein in its entirety.

### GOVERNMENT SUPPORT

This invention was made with Government support under Grant No. P50 GM098792 awarded by the National Institutes of Health, and Grant No. N00014-13-1-0074 awarded by the Office of Naval Research. The Government has certain rights in the invention.

### BACKGROUND

Since its adaptation for site-specific DNA cleavage, the CRISPR-endonuclease system has been widely used for genome editing in a variety of organisms, from prokaryotes to eukaryotes (Hsu et al. (2014) Cell 157(6):1262-78; Sander et al. (2014) Nat Biotechnol 32(4):347-55). Researchers have generated catalytically inactive endonucleases that retain the ability to bind DNA but lack endonucleolytic activity to repress expression of target genes (Qi et al. (2013) Cell 152(5):1173-83).

### SUMMARY

The present disclosure relates to the rational design of complex cellular behaviors using RNA-guided genetic circuits. Living cells naturally sense and respond to environmental signals using complex genetically-encoded “circuits” that result in complex and adaptive behaviors (FIG. 1). As is disclosed herein, catalytically inactive RNA-guided endonuclease systems (e.g., dCas9, dCpf1, etc.) provide a potentially scalable method to engineer cellular behaviors. In these systems, an RNA-guided endonuclease (e.g., Cas9, Cpf1, etc.) is mutated so that it no longer cleaves DNA. A specific guide RNA targets the catalytically inactive endonuclease to a specific genetic locus in response to one or more input signals. The catalytically inactive endonuclease binds to the target locus and reduces or blocks transcription of one or more output sequences.

In some embodiments, if an output sequence is transcribed, the output sequence in turn becomes one or more input signals that control the transcription of one or more downstream output sequences, thereby allowing the formation of layered genetic circuits that control increasingly complex cellular behaviors.

The genetic circuits disclosed herein comprise a heterologous polymerase (e.g., T7 RNA polymerase) that is able to activate transcription of promoter/operators across different species. The use of heterologous polymerases instead of the sole reliance on the host RNA polymerase to transcribe components of the genetic circuit overcomes organism-dependent functionality of the genetic circuit and provides the additional advantage of increased versatility.

According to one aspect, a method of controlling expression of a first output sequence is provided herein. In some embodiments, the method of controlling expression of a first output sequence includes introducing into a cell a genetic circuit that includes one or more polynucleotide sequences, wherein the genetic circuit includes: (a) a first output sequence; (b) a first promoter/operator controlling transcription of the first output sequence; (c) a first guide RNA targeting the first promoter/operator; (d) a second promoter/operator controlling transcription of the first guide RNA, wherein the second promoter/operator is input-sensitive such that a first input signal is required for induction of transcription of the first guide RNA; (e) a first catalytically inactive endonuclease that in combination with the first guide RNA binds to a sequence targeted by the first guide RNA and prevents transcription of the first output sequence; (f) a third promoter/operator controlling transcription of the first catalytically inactive endonuclease, wherein the third promoter/operator is input-sensitive such that a second input signal is required for induction of transcription of the first catalytically inactive endonuclease; and (g) one or more heterologous polymerases that specifically bind one or more of the first, second and/or third promoter/operator.

In some embodiments, the genetic circuit further includes a fourth promoter/operator controlling transcription of one or more second output sequence, wherein the fourth promoter/operator is input-sensitive such that a third input signal is required for induction of transcription of the one or more second output sequence.

In some embodiments, the genetic circuit further includes one or more endogenous polymerases that bind one or more of the first, second, third and/or fourth promoter/operator.

In some embodiments, the third input signal is one or more second guide RNA encoded by the first output sequence and wherein the one or more second output sequence is one or more third guide RNA.

In some embodiments, at least two of the first, second and third input signals are the same. In some embodiments, any of the first, second, third and/or fourth promoter/operator includes a T7 promoter and an operator.

In some embodiments, the heterologous polymerase is a viral polymerase. In certain embodiments, the heterologous polymerase is a T7 RNA polymerase.

In some embodiments, the genetic circuit further includes polynucleotide sequences encoding one or more decoy operators having the same, or substantially the same, sequence as one or more of the operator sequences of the first, second, third and/or fourth promoter/operators.

In some embodiments, the genetic circuit further includes one or more polynucleotide sequences encoding one or more small RNAs (sRNAs) that binds to and sequesters the guide RNA.

In some embodiments, the first, second and/or third guide RNA is a nested guide RNA including two or more sequences that target two or more target sequences in the first, second, third and/or fourth promoter/operator, thereby causing promoter looping of the first, second, third and/or fourth promoter/operator.

In some embodiments, the first, second and/or third input signal is a chemical, light, a polypeptide or a mechanical force. In certain embodiments, the first, second and/or third input signal is isopropyl β-D-1-thiogalactopyranoside (IPTG), anhydrotetracycline (aTc), or 2,4-diacetylphloroglucinol (DAPG).

In some embodiments, the first and/or second output sequence encode one or more first output molecule and the one or more first output molecule in turn becomes a fourth input signal required for controlling transcription of one or more third output sequence. In certain embodiments, the first second and/or third output sequence is a fourth guide RNA.

In some embodiments, two or more input signals control transcription in a single promoter/operator.

In some embodiments, the first catalytically inactive endonuclease is a RNA-guided DNA endonuclease. In some embodiments, the first catalytically inactive endonuclease is a catalytically inactive clustered regularly interspaced short palindromic repeat (CRISPR) endonuclease. In some embodiments, the first catalytically inactive endonuclease is catalytically inactive Cas9 or catalytically inactive Cpf1. In certain embodiments, the first catalytically inactive endonuclease is selected from the group consisting of dSpCas9, dSpCas9(E), dSpCas9(VRER), dSpCas9(VQR), dSpCas9(EQR), desSpCas9, dSpCas9-HF1, dSaCas9, desSaCas9, dSt1Cas9, dFnCpf1, dAsCpf1, and dLbCpf1.

In some embodiments, the one or more nucleotide of the first, second, third and/or fourth guide RNA is mutated and the mutation of the first, second, third and/or fourth guide RNA does not decrease prevention of transcription of the first, second and/or third output sequence.

In some embodiments, the cell is a prokaryotic cell. In some embodiments, the cell is a bacterial cell. In some embodiments, the genetic circuit in the bacterial cell is optimized for bioreactor growth. In some embodiments, the cell is part of a microbiome. In certain embodiments, the cell is a BL21(DE3) cell.

In some embodiments, the cell is a eukaryotic cell. In certain embodiments, the cell is a plant cell. In certain embodiments, the cell is a human cell.

In some embodiments, the first, second, third and/or fourth promoter/operator is sensitive to a guide RNA. In some embodiments, the first, second, third and/or fourth promoter/operator is sensitive to a chemical input.

In some embodiments, the first, second, third and/or fourth promoter/operator includes a polynucleotide sequence that encodes a T7 promoter and a polynucleotide sequence that encodes a PhlF operator.

In some embodiments, the T7 promoter and the PhlF operator control the transcription of guide RNA A2NT. In some embodiments, the guide RNA A2NT controls the transcription of the A2NT operator.

In some embodiments, the first, second and/or third output sequence is a DNA sequence. In some embodiments, the first, second and/or third output sequence encodes one or more second output molecule.

In some embodiments, the first and/or second output molecule controls a fifth heterologous promoter/operator controlling transcription of one or more fourth output sequence. In some embodiments, the first and/or second output molecule is a protein.

In some embodiments, the methods described herein further include culturing the cell under conditions that allow expression of the genetic circuit in the cell.

According to another aspect, a genetic circuit is provided herein. In some embodiments, the genetic circuit includes: (a) a first output sequence; (b) a first promoter/operator controlling transcription of the first output sequence; (c) a first guide RNA targeting the first promoter/operator; (d) a second promoter/operator controlling transcription of the first guide RNA, wherein the second promoter/operator is input-sensitive such that a first input signal is required for induction of transcription of the first guide RNA; (e) a first catalytically inactive endonuclease that in combination with the first guide RNA binds to a sequence targeted by the first guide RNA and prevents transcription of the first output sequence; (f) a third promoter/operator controlling transcription of the first catalytically inactive endonuclease, wherein the third promoter/operator is input-sensitive such that a second input signal is required for induction of transcription of the first catalytically inactive endonuclease; and (g) one or more heterologous polymerases that specifically bind one or more of the first, second and/or third promoter/operator.

In some embodiments, the genetic circuit further includes a fourth promoter/operator controlling transcription of one or more second output sequence, wherein the fourth promoter/operator is input-sensitive such that a third input signal is required for induction of transcription of the one or more second output sequence.

In some embodiments, the genetic circuit described herein further includes one or more endogenous polymerases that bind one or more of the first, second, third and/or fourth promoter/operator.

In some embodiments, the third input signal is one or more second guide RNA encoded by the first output sequence and wherein the one or more second output sequence is one or more third guide RNA. In some embodiments, at least two of the first, second and third input signals are the same. In some embodiments, any of the first, second, third and/or fourth promoter/operators includes a T7 promoter and an operator.

In some embodiments, the heterologous polymerase is a viral polymerase. In certain embodiments, the heterologous polymerase is a T7 RNA polymerase.

In some embodiments, the genetic circuit further includes polynucleotide sequences encoding one or more decoy operators having the same, or substantially the same, sequence as one or more of the operator sequences of the first, second, third and/or fourth promoter/operators.

In some embodiments, the genetic circuit further includes one or more polynucleotide sequences encoding one or more small RNAs (sRNAs) that binds to and sequesters the guide RNA.

In some embodiments, the first, second, and/or third guide RNA is a nested guide RNA comprising two or more sequences that target two or more target sequences in the first, second, third and/or fourth promoter/operator, thereby causing promoter looping of the first, second, third and/or fourth promoter/operator.

In some embodiments, the first, second and/or third input signal is a chemical, light, a polypeptide or a mechanical force. In certain embodiments, the first, second and/or third input signal is IPTG, aTc, or DAPG.

In some embodiments, the first and/or second output sequence encode one or more first output molecule and the one or more first output molecule in turn becomes a fourth input signal required for controlling transcription of one or more third output sequences. In certain embodiments, the first, second and/or third output sequence is a fourth guide RNA.

In some embodiments, two or more input signals control transcription in a single promoter/operator.

In some embodiments, the first catalytically inactive endonuclease is a RNA-guided DNA endonuclease.

In some embodiments, the first catalytically inactive endonuclease is a catalytically inactive CRISPR endonuclease. In some embodiments, the first catalytically inactive endonuclease is catalytically inactive Cas9 or catalytically inactive Cpf1. In certain embodiments, the first catalytically inactive endonuclease is selected from the group consisting of dSpCas9, dSpCas9(E), dSpCas9(VRER), dSpCas9(VQR), dSpCas9(EQR), desSpCas9, dSpCas9-HF1, dSaCas9, desSaCas9, dSt1Cas9, dFnCpf1, dAsCpf1, and dLbCpf1.

In some embodiments, one or more nucleotide of the first, second, third and/or fourth guide RNA is mutated and the mutation of the first, second, third and/or fourth guide RNA does not decrease prevention of transcription of the first, second and/or third output sequence.

In some embodiments, the first, second, third and/or fourth promoter/operator is sensitive to a guide RNA. In some embodiments, the first, second, third and/or fourth promoter/operator is sensitive to a chemical input.

In some embodiments, the first, second, third and/or fourth promoter/operator includes a polynucleotide sequence that encodes a T7 promoter and a polynucleotide sequence that encodes a PhlF operator. In some embodiments, the T7 promoter and the PhIF operator control the transcription of guide RNA A2NT. In certain embodiments, the guide RNA A2NT controls the transcription of the A2NT operator.

In some embodiments, one or more of the first, second and/or third output sequence is a DNA sequence. In some embodiments, the first, second and/or third output sequence encodes a second output molecule.

In some embodiments, the first and/or second output molecule controls a fifth heterologous promoter/operator controlling transcription of one or more fourth output sequences. In some embodiments, the first and/or second output molecule is a protein.

The subject matter of this application may involve, in some cases, interrelated products, alternative solutions to a particular problem, and/or a plurality of different uses of a single system or article. Other advantages, features, and uses will be apparent from the detailed description of certain non-limiting embodiments, the drawings, and the claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1 is a schematic showing user-designed cellular behavior using a programming language and software. An RNA-guided genetic circuit that can sense signals, process that information, and drive complex output behaviors is automatically designed by the software. Circuits are composed of gates (symbols in box), which are automatically compiled to a genetic layout and DNA sequence. This figure depicts SEQ ID NO:28.

FIGS. 2A-2B show that different dCas9 and dCpf1 proteins vary in their ability to repress transcription, and also their toxicity. These parameters were measured for 14 Cas9/Cpf1 variants (FIG. 2A), and are shown on a plot (FIG. 2B). The upper left quadrant of FIG. 2B contains variants with useful properties for genetic circuits.

FIGS. 3A-3B show that guide RNA gates are normally non-cooperative, eliciting “analog” input-output response curves (light gray line). The addition of increasing numbers of decoy operator sites increases the non-linearity of the response curve (the results from progressively higher numbers of decoy operators are shown from lighter to darker gray lines in FIG. 3A). Similarly, degradation of guide RNA by a constitutively produced small RNA (sRNA) also introduces non-linearity into the system (dark gray line, FIG. 3B).

FIGS. 4A-4B show a proof-of-concept for organism independent RNA guided genetic circuits. Traditional approaches to implementing RNA guided genetic circuits use the transcriptional machinery of the host organism to drive each gate. This requires that a given circuit must be redesigned for each organism, and quantitative information about the strength of transcription may not be available. By driving each gate with a heterologous T7 RNA polymerase (RNAP), the entire transcriptional cascade of a genetic circuit can be processed using non-native machinery (FIG. 4A). This simplifies the design of circuits across organisms. Proof-of-concept data is shown in FIG. 4A-4B and for repressing a T7 RNAP-driven gate in a strain that produces T7 RNAP.

FIG. 5 shows software that automates the design of RNA-guided genetic circuits. A user inputs a behavioral specification using a high-level hardware description language along with sensor input information (left panel). The left panel depicts SEQ ID NOs: 29 through 31 from top to bottom, respectively. Algorithms synthesize a circuit diagram, assign biochemical gates, and generate one or more genetic layouts for the circuit (middle panel). The DNA sequence is output in a specified genetic context, and simulations of circuit performance are provided (right panel).

FIGS. 6A-6B show gRNA-gate engineering and measurement.

FIG. 7 shows mutational scanning of guide RNAs that reveals sites for diversification: AsCpf1. The nucleotide sequence on the top heatmap of this figure is SEQ ID NO:32.

FIG. 8 shows mutational scanning of guide RNAs that reveals sites for diversification: SpCas9. The nucleotide sequence on the top heatmap of this figure is SEQ ID NO:33. The top and bottom nucleotide sequences on the bottom heatmap correspond to the following portions of SEQ ID NO: 33: 1 and 1′, 2 and 2′, 3 and 3′, 4 and 4′, and 5 and 5′.

FIG. 9 shows mutational scanning of guide RNAs that reveals sites for diversification: SaCas9. The nucleotide sequence on the top heatmap of this figure is SEQ ID NO:34. The top and bottom nucleotide sequences on the bottom heatmap correspond to the following portions of SEQ ID NO: 34: 1 and 1′, 2 and 2′, 3 and 3′, 4 and 4′.

FIG. 10 shows mutational scanning of guide RNAs that reveals sites for diversification: St1Cas9. The nucleotide sequence on the top heatmap of this figure is SEQ ID NO:35. The top and bottom nucleotide sequences on the bottom heatmap correspond to the following portions of SEQ ID NO: 35: 1 and 1′, 2 and 2′, 3 and 3′, 4 and 4′.

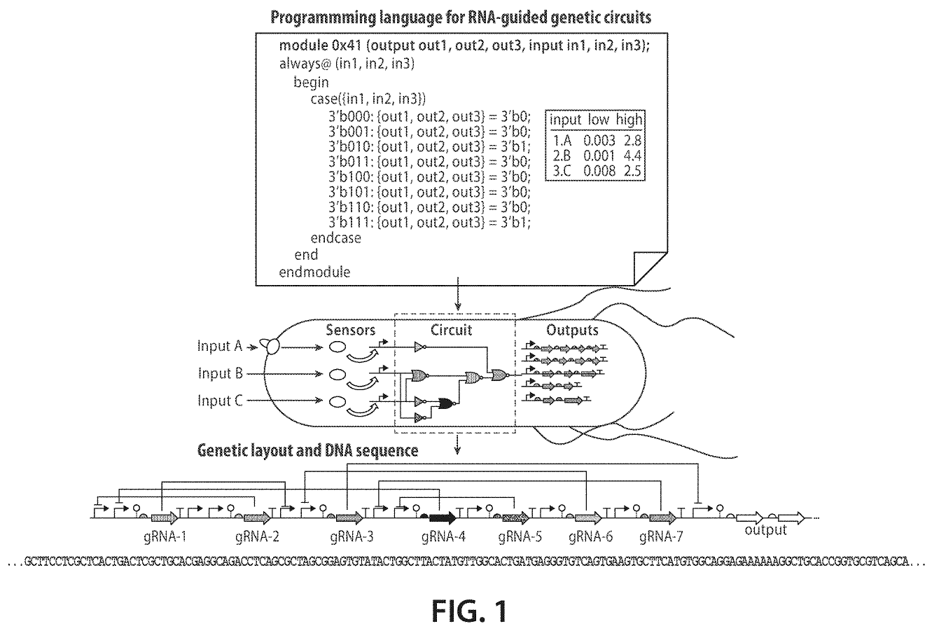

### DETAILED DESCRIPTION

CRISPR technology has been widely applied for genome editing and modulation. The ease of engineering of the guide RNA of the CRISPR system makes it an attractive platform for generating synthetic genetic circuits. Provided herein are novel genetic circuits engineered to express one or more output sequences, which may be employed, for example, for controlling complex cellular behaviors. As disclosed herein, the genetic circuits employ catalytically-inactive endonuclease systems (e.g., RNA-guided endonuclease systems, including but not limited to dCas9, dCpf1, etc.) to provide a scalable methods to engineer cellular behaviors. In these systems, an RNA-guided endonuclease (e.g., Cas9, Cpf1, etc.) is mutated so that it no longer cleaves DNA (e.g., dCas9, dCpf1, etc.) and a specific guide RNA targets the catalytically-inactive endonuclease to a specific genetic locus, such as a promoter/operator, which reduces or blocks transcription of a nucleic acid sequence operably linked to the genetic locus. Transcription of the catalytically inactive endonuclease and, in some embodiments, the RNA guide are under the control of a promoter/operator sequence recognized by a heterologous polymerase. In some embodiments, the promoter is a T7 promoter that is recognized by the bacteriophage T7 RNA polymerase.

The genetic circuits provided herein can connect a sensory input signal to the transcription of a guide RNA, and use the guide RNA to target the catalytically inactive endonuclease to sequence controlling expression of a specific output gene to cause transcriptional repression (the input-output unit is, in some cases, termed a “gate”). Provided herein are increasingly complex circuits constructed by, for example, connecting the output of one gate to the input of another gate (e.g., layering), by driving a gate using multiple inputs instead of one, and by splitting an output to drive multiple downstream gates. The genetic circuits provided herein have several applications in biotechnology, including but not limited to, control of therapeutic molecule synthesis and administration (e.g., production of non-ribosome peptides, modulation of T-cell activity, pancreatic cell molecule production, etc.), use in agriculture (e.g., expression of drought- or cold-resistance genes, expression of herbicide or pesticide genes, expression of genes that affect the nutritional profile, etc.), and production of materials (e.g., spider silk, magnetic nanoparticles, silica structures, etc.).

Several challenges exist for RNA-guided genetic circuits, including toxicity of biomolecular components, linear (or “non-cooperative”) gate responses that are highly-sensitive to noise and perturbations, repeated use of DNA sequences that can cause genetic deletions, and organism-dependent circuit functionality. The genetic circuits disclosed herein comprise a heterologous polymerase that is able to activate transcription of promoter/operators across different species. The use of heterologous polymerase system, instead of the host RNA polymerase, overcomes organism-dependent functionality of the genetic circuit and provides the additional advantage of increased versatility.

Aspects of the present disclosure relate to genetic circuits and methods of using said genetic circuits for controlling the expression of one or more output sequences and complex cellular behaviors.

For clarity and ease of explanation, input signals, output sequences, output molecules or promoter/operators may be referred to as first, second, third or fourth input signal, output sequence, output molecule or promoter/operator (and so on) so as to distinguish one input signal, output sequence, output molecule or promoter/operator from another input signal, output sequence, output molecule or promoter/operator.

In some embodiments, the genetic circuit described herein includes a first promoter/operator sequence operably linked to a nucleotide sequence that controls transcription of a first output sequence. The activity of the first promoter/operator is controlled by a first guide RNA, which binds to a first catalytically inactive endonuclease and guides the first catalytically inactive endonuclease to the first promoter/operator sequence to block transcription of the output sequence. The transcription of the first guide RNA is controlled by a second promoter/operator sequence operably linked to a nucleotide sequence that encodes the first guide RNA. A first input signal activates the second promoter/operator, resulting in production of the first guide RNA. The transcription of the first catalytically inactive endonuclease is controlled by a third promoter/operator sequence that is operably linked to the nucleotide sequence that encodes the first catalytically inactive endonuclease. A second input signal activates the third promoter/operator, resulting in production of the first catalytically inactive endonuclease. Therefore, one layer of regulation by the genetic circuit contemplated herein includes one or more input signals that activate the second and third promoter/operator sequences, which, in turn, transcribe the first guide RNA and the first catalytically inactive endonuclease, respectively. The first guide RNA binds to the first catalytically inactive endonuclease, targets the catalytically inactive endonuclease to a specific sequence in the first promoter/operator sequence, and reduces, or in some embodiments, blocks transcription of the first output sequence.

Two or more layers of regulation by the genetic circuit are also contemplated herein. In some embodiments, if the first output sequence is transcribed, the first output sequence encoded by the first promoter/operator is a second guide RNA. The second guide RNA binds to and guides the first and/or a second catalytically inactive endonuclease to a specific sequence in a fourth promoter/operator operably linked to a nucleotide sequence that encodes one or more second output sequences. The first and/or second catalytically inactive endonuclease then binds to a specific sequence in the fourth promoter/operator sequence and reduces or, in some embodiments, blocks transcription of the one or more second output sequences. In certain embodiments, if the one or more second output sequence is transcribed, the one or more second output sequences are one or more third guide RNAs that control transcription of one or more additional output sequences (e.g., third, fourth, fifth, etc.) under the control of one or more additional promoter/operators (e.g., fifth, sixth, seventh, etc.). Therefore, the first output sequence is, for instance, a guide RNA that binds to and guides the first and/or second catalytically inactive endonuclease to a fourth promoter/operator and blocks transcription of one or more second output sequences. In certain embodiments, the first and/or second output sequences are coding RNAs that are translated into one or more output molecules (e.g., proteins, such as therapeutic proteins). In other embodiments, the first output sequence and the one or more second output sequence is a non-coding RNA (e.g., rRNA, tRNA, small nuclear RNA (snRNA), small nucleolar RNA (snoRNA), miRNA, small RNA that sequesters the guide RNA, guide RNA, etc.). Additional layers of regulation are also contemplated herein.

In certain embodiments, the catalytically inactive endonuclease guided by the guide RNA is targeted to a region downstream of the promoter/operator. In certain embodiments, the catalytically inactive endonuclease guided by the guide RNA is targeted to the coding sequence of the one or more output sequences.

At a given time, a first input signal activates a first input-sensitive promoter/operator that transcribes a first guide RNA, while a second input signal activates a second input-sensitive promoter/operator that expresses a first catalytically inactive endonuclease. Therefore, in the presence of the first and second input signals, the first guide RNA binds to and guides the first catalytically inactive endonuclease to a target third promoter/operator sequence and reduces or, in some embodiments, blocks transcription of a first output sequence (e.g., second guide RNA, etc.) and/or first output molecule (e.g., protein, etc.). Therefore, if the first output sequence is a second guide RNA, transcription of the second guide RNA is reduced or blocked. Reduced or blocked transcription of the second guide RNA reduces or abolishes its availability to bind to and guide the first and/or a second catalytically inactive endonuclease to a target fourth promoter/operator and reduce or block transcription. In this case, the fourth promoter/operator is able to transcribe one or more output sequences. Conversely, in the absence of the first input signal, the first guide RNA is not transcribed. Similarly, in the absence of the second input signal the catalytically inactive endonuclease is not transcribed and expressed. Therefore, in the absence of the first and/or second input signals, the activity of the first promoter/operator is not reduced or blocked and the first output sequence (e.g., second guide RNA, etc.) and/or first output molecule (e.g., proteins, etc.) is transcribed. A similar effect would result from the presence of the first input signal and the absence of the second input signal, and vice versa.

As observed from the example above, additional layers of control can be readily added to the genetic circuits contemplated herein. Therefore, the versatility of the genetic circuit allows for the control of complex networks and cellular behaviors.

The genetic circuits described herein for controlling the expression of one or more output sequences, which, in turn, can be used to control one or more cellular behaviors, involve several components, as described below.

Promoter

As used herein, a “promoter” is a control region of a nucleic acid at which initiation and rate of transcription of the remainder of a nucleic acid are controlled. As used herein, the term “promoter/operator” is used to describe a promoter in proximity or next to an operator sequence. A promoter and/or promoter/operator may also contain sub-regions at which regulatory proteins and molecules may bind, such as RNA polymerase and other transcription factors. The promoter and/or promoter/operator and/or any additional regulatory sequences said to be “operably” joined to another nucleic acid sequence when they are covalently linked in such a way as to place the expression or transcription of the coding sequence under the influence or control of the regulatory sequences. If it is desired that the coding sequences be transcribed into an output sequence (e.g., guide RNA) and/or output molecule (e.g., protein), two DNA sequences are said to be “operably linked” if induction of a promoter and/or promoter/operator in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences.

Promoters and/or promoter/operators may be constitutive, inducible, activatable, repressible, tissue-specific, cell-specific, cell-state specific, or any combination thereof. Any promoter and/or promoter/operators known in the art may be used to control the expression of the output sequence(s). In some embodiments, the promoter is recognized by the T7 RNA polymerase. A promoter and/or promoter/operator may be one naturally associated with a gene or sequence, as may be obtained by isolating the 5′ non-coding sequences located upstream of the coding segment of a given gene or sequence.

As used herein, a “recombinant promoter,” “recombinant promoter/operator,” “heterologous promoter,” or “heterologous promoter/operator” is a promoter or promoter/operator that is not naturally or normally associated with or that does not naturally or normally control transcription of a DNA sequence to which it is operably linked. In some embodiments, a nucleotide sequence (e.g., an output sequence that encodes a guide RNA) may be positioned under the control of a recombinant promoter, recombinant promoter/operator, heterologous promoter or heterologous promoter/operator. Such promoters or promoter/operators may include promoters or promoter/operators of other genes; promoters or promoter/operators isolated from any prokaryotic cell; and synthetic promoters or promoter/operators that are not “naturally occurring”, such as those that contain different elements of different transcriptional regulatory regions and/or mutations that alter expression as compared to non-mutated forms of the regulatory regions.

In some embodiments, the promoter is a prokaryotic promoter. In some embodiments, the prokaryotic promoter in the promoter/operator is a T7 promoter. In other embodiments, the promoter in the promoter/operator is a eukaryotic promoter. Non-limiting examples of prokaryotic (Table 1) and eukaryotic promoters (Table 2) known to those of ordinary skill in the art (see, e.g., Addgene website: blog.addgene.org/plasmids-101-the-promoter-region) are shown below.

TABLE 1
Exemplary Prokaryotic Promoters

| Promoter | Primary Use | Description | Expression |
|---|---|---|---|
| T7 | In vitro transcription/ | Promoter from T7 | Constitutive, but requires T7 |
| | general expression | bacteriophage | RNA polymerase. |
| T7lac | High levels of gene | Promoter from T7 | Negligible basal expression |
| | expression | bacteriophage plus | when not induced. Requires T7 |
| | | lac operators | RNA polymerase, which is also |
| | | | controlled by lac operator. Can |
| | | | be induced by IPTG. |
| Sp6 | in vitro transcription/ | Promoter from Sp6 | Constitutive, but requires SP6 |
| | general expression | bacteriophage | RNA polymerase. |
| araBAD | General expression | Promoter of the | Inducible by arabinose and |
| | | arabinose metabolic | repressed catabolite repression |
| | | operon | in the presence of glucose or by |
| | | | competitive binding of the anti- |
| | | | inducer fucose |
| trp | High levels of gene | Promoter from E. coli | Repressible |
| | expression | tryptophan operon | |
| lac | General expression | Promoter from lac | Constitutive in the absense of |
| | | operon | lac repressor (lacI or lacIq). Can |
| | | | be induced by IPTG or lactose. |
| Ptac | General expression | Hybrid promoter of | Regulated like the lac promoter |
| | | lac and trp | |
| pL | High levels of gene | Promoter from | Can be temperature regulatable |
| | expression | bacteriophage lambda | |

TABLE 2
Exemplary Eukaryotic Promoters

| | Primary | RNA | | |
|---|---|---|---|---|
| Promoter | Use | Transcript | Description | Expression |
| CMV | General | mRNA | Strong mammalian expression | Constitutive |
| | expression | | promoter from the human | |
| | | | cytomegalovirus | |
| EF1a | General | mRNA | Strong mammalian expression from | Constitutive |
| | expression | | human elongation factor 1 alpha | |
| SV40 | General | mRNA | Mammalian expression promoter | Constitutive |
| | expression | | from the simian vacuolating virus 40 | |
| PGK1 | General | mRNA | Mammalian promoter | Constitutive |
| (human or | expression | | from phosphoglycerate kinase gene. | |
| mouse) | | | | |
| Ubc | General | mRNA | Mammalian promoter from the | Constitutive |
| | expression | | human ubiquitin C gene | |
| human | General | mRNA | Mammalian promoter from beta | Constitutive |
| beta actin | expression | | actin gene | |
| CAG | General | mRNA | Strong hybrid mammalian promoter | Constitutive |
| | expression | | | |
| TRE | General | mRNA | Tetracycline response element | Inducible with |
| | expression | | promoter | Tetracyline/derivatives |
| UAS | General | mRNA | Drosophila promoter conaining Gal4 | Specific |
| | expression | | binding sites | |
| Ac5 | General | mRNA | Strong insect promoter from | Constitutive |
| | expression | | Drosophila Actin 5c gene | |
| Polyhedrin | General | mRNA | Strong insect promoter from | Constitutive |
| | expression | | baculovirus | |
| CaMKIIa | Gene | mRNA | Ca2+/calmodulin-dependent protein | Specific |
| | expression | | kinase II promoter | |
| | for | | | |
| | optogenetics | | | |
| GAL1, 10 | General | mRNA | Yeast adjacent, divergently | Inducible with |
| | expression | | transcribed promoters | galactose; |
| | | | | repressible with |
| | | | | glucose |
| TEF1 | General | mRNA | Yeast transcription elongation factor | Constitutive |
| | expression | | promoter | |
| GDS | General | mRNA | Strong yeast expression promoter | Constitutive |
| | expression | | from glyceraldehyde 3-phosphage | |
| | | | dehydrogenase | |
| ADH1 | General | mRNA | Yeast promoter for alcohol | Repressed |
| | expression | | dehydrogenase I | by ethanol |
| CaMV35S | General | mRNA | Strong plant promoter from the | Constitutive |
| | expression | | Cauliflower Mosaic Virus | |
| Ubi | General | mRNA | Plant promoter from maize ubiquitin | Constitutive |
| | expression | | gene | |
| H1 | Small RNA | shRNA | From the human polymerase III | Constitutive |
| | expression | | RNA promoter | |
| U6 | Small RNA | shRNA | From the human U6 small nuclear | Constitutive |
| | expression | | promoter | |

Additional non-limiting examples of promoters or promoter/operators known to those of ordinary skill in the art include, without limitation, bacteriophage promoters (e.g. P1s1con, T3, T7, SP6, PL), preferably the T7 promoter, and bacterial promoters (e.g. Pbad, PmgrB, Ptrc2, Plac/ara, Ptac, Pm), or hybrids thereof (e.g. PLlacO, PLtetO). Examples of bacterial promoters for use in accordance with the present disclosure include, without limitation, positively regulated E. coli promoters such as positively regulated σ70 promoters (e.g., inducible pBad/araC promoter, Lux cassette right promoter, modified lamdba Prm promote, plac Or2-62 (positive), pBad/AraC with extra REN sites, pBad, P(Las) TetO, P(Las) CIO, P(Rhl), Pu, FecA, pRE, cadC, hns, pLas, pLux), σS promoters (e.g., Pdps), σ32 promoters (e.g., heat shock) and σ54 promoters (e.g., glnAp2); negatively regulated E. coli promoters such as negatively regulated σ70 promoters (e.g., Promoter (PRM+), modified lamdba Prm promoter, TetR-TetR-4C P(Las) TetO, P(Las) CIO, P(Lac) IQ, RecA_DlexO_DLacO1, dapAp, FecA, Pspac-hy, pcI, plux-cI, plux-lac, CinR, CinL, glucose controlled, modified Pr, modified Prm+, FecA, Pcya, rec A (SOS), Rec A (SOS), EmrR_regulated, BetI_regulated, pLac_lux, pTet_Lac, pLac/Mnt, pTet/Mnt, LsrA/cI, pLux/cI, LacI, LacIQ, pLacIQ1, pLas/cI, pLas/Lux, pLux/Las, pRecA with LexA binding site, reverse BBa_R0011, pLacI/ara-1, pLacIq, rrnB P1, cadC, hns, PfhuA, pBad/araC, nhaA, OmpF, RcnR), σS promoters (e.g., Lutz-Bujard LacO with alternative sigma factor σ38), σ32 promoters (e.g., Lutz-Bujard LacO with alternative sigma factor σ32), and σ54 promoters (e.g., glnAp2); negatively regulated B. subtilis promoters such as repressible B. subtilis σA promoters (e.g., Gram-positive IPTG-inducible, Xyl, hyper-spank) and σB promoters. Other inducible promoters known in the art may be used in accordance with the present disclosure.

A promoter or promoter/operator is considered “input-sensitive” to an input signal if the input signal modulates the function of the promoter, directly or indirectly. In some aspects, the input signal initiates or enhances transcriptional activity of the promoter when the promoter is in the presence of, influenced by, or contacted by the input signal. In some embodiments, an input signal may positively modulate a promoter such that the promoter activates, or increases (e.g., by a certain percentage or degree), transcription of a nucleic acid to which it is operably linked. In some embodiments, by contrast, an input signal may negatively modulate a promoter such that the promoter or promoter/operator is prevented from activating or inhibits, or decreases, transcription of a nucleic acid to which it is operably linked. An input signal may modulate the function of the promoter or promoter/operator directly by binding to the promoter or promoter/operator or by acting on the promoter or promoter/operator without an intermediate signal.

For clarity and ease of explanation, promoter/operators responsive to an input signal, output sequence or output molecule, may be referred to as first, second, third or fourth promoter/operators (and so on) so as to distinguish one promoter/operator from another. It should be understood that reference to a first promoter/operator and a second promoter/operator may encompass two different promoter/operators (e.g., P_{T7}/PhIH v. P_{T7}/A2NT). However, in some embodiments, the first promoter/operators and second promoter/operators are the same but can be rendered differentially responsive by other regulatory element(s) used in combination with the promoter/operators.

In accordance with the present disclosure, an input-sensitive promoter includes, without limitation, chemically/biochemically-regulated and physically-regulated promoters, such as alcohol-regulated, 2,4-diacetylphloroglucinol (DAPG)-regulated, isopropyl β-D-1-thiogalactopyranoside (IPTG)-regulated, tetracycline-regulated, steroid-regulated, metal-regulated, pathogenesis-regulated, temperature/heat-inducible and light-regulated promoters.

Tetracycline-regulated promoters include, without limitation, anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA).

Steroid-regulated promoters include, without limitation, promoters based on the rat glucocorticoid receptor, human estrogen receptor, moth ecdysone receptors, and promoters from the steroid/retinoid/thyroid receptor superfamily.

Metal-regulated promoters include, without limitation, promoters derived from metallothionein (proteins that bind and sequester metal ions) genes from yeast, mouse and human.

Pathogenesis-regulated promoters include, without limitation, promoters induced by salicylic acid, ethylene or benzothiadiazole (BTH).

Temperature/heat-inducible promoters include, without limitation, heat shock promoters.

Light-regulated promoters include, without limitation, light responsive promoters from plant cells.

An input-sensitive promoter for use in accordance with the present disclosure may be induced by (or repressed by) one or more physiological condition(s), such as changes in pH, temperature, radiation, osmotic pressure, saline gradients, cell surface binding, and the concentration of one or more extrinsic or intrinsic inducing agent(s). The extrinsic inducer or inducing agent may comprise, without limitation, amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or combinations thereof.

Input Signal

An “input signal” may be endogenous or a normally exogenous condition, compound or protein that contacts a programmable endonuclease circuit in such a way as to change transcriptional activity from the input-sensitive promoter. As used herein an input signal refers to any chemical (e.g., signals extrinsic or intrinsic to a cell, such as amino acids and amino acid analogs, saccharides and polysaccharides, nucleic acids, protein transcriptional activators and repressors, cytokines, toxins, petroleum-based compounds, metal containing compounds, salts, ions, enzymes, enzyme substrates, enzyme substrate analogs, hormones, quorum-sensing molecules, proteins, small molecules (e.g., IPTG, DAPG, aTc), catalytically-inactive endonucleases and guide RNAs) or non-chemical (e.g., pH, light, temperature, heat, radiation, osmotic pressure, saline gradients, and mechanical force) signal in a cell, or to which the cell is exposed, that modulates, directly or indirectly, a component (e.g., a promoter) of a genetic circuit. In some embodiments, an input signal is a biomolecule that modulates the function of a promoter (referred to as direct modulation), or is a signal that modulates a biomolecule, which then modulates the function of the promoter (referred to as indirect modulation). In some embodiments, an output signal is an output sequence or output molecule (e.g. guide RNA, protein, etc.) For clarity and ease of explanation, an input signal may be referred to as first, second, third or fourth input signal (and so on) so as to distinguish one input signal from another input signal.

As used herein, the input-output unit is termed a “gate”. In certain aspects, the genetic circuit is designed to detect and to generate a response to one or more input signals. For example, a gate may detect and generate a response to 2, 3, 4, 5, 6, 7, 8, 9 or 10 or more input signals.

According to certain aspects, the genetic circuit, output sequences or output molecules expressed by the genetic circuit, is delivered to a subject (e.g., a human subject) using, for example, in bacteriophage or phagemid vehicles, or other delivery vehicle that is capable of delivering nucleic acids to a cell in vivo. In some embodiments, a genetic circuit may be introduced into cells ex vivo, which cells are then delivered to a subject via injection, oral delivery, or other delivery route or vehicle. Other uses of genetic circuits are contemplated by the present disclosure. For example, the present disclosure provides the rational design of cells with programmed cellular behaviors to control microbiome therapeutics to sense and respond to disease, bacterial strains optimized for bioreactor growth and chemical production, “smart” plants that can modulate gene expression in drought conditions, and materials which can grow and heal themselves.

RNA Polymerase

The genetic circuits contemplated herein employ RNA polymerases for transcription of target sequences (e.g., guide RNA or output sequences). In some embodiments, the genetic circuits employ the polymerase in the native cellular transcription machinery. In some embodiments, the genetic circuits contemplated herein employ a “heterologous polymerase.” As used herein, the term “heterologous polymerase” refers to a polymerase that is not part of the native cellular transcription machinery or is not “naturally occurring”. In some embodiments, the heterologous polymerase is of viral origin. In certain embodiments, the heterologous polymerase is a bacteriophage T7 RNA polymerase. In certain embodiments, the genetic circuits described herein comprises a T7 RNA polymerase and a T7 promoter/operator controlling transcription of one or more output sequences, wherein the T7 RNA induces transcription of the one or more output sequences without the use of the native cellular transcription machinery. In some embodiments, the genetic circuits described herein use the native cellular transcription machinery in addition to a heterologous transcription machinery.

The RNA polymerases specified by viruses, such as the bacteriophages T7, which is in some embodiments part of the genetic circuits described herein, and related family members T3 and SP6 are single-subunit enzymes that are highly specific for their cognate promoters and terminators and are able to maintain rapid rates of transcription in vitro (McClure (1985) Annu Rev Biochem 54, 171-204). The family is also related to the mitochondrial RNA polymerase. The elongation rate for T7 RNA polymerase (RNAP), for example, has been measured at ˜230 nucleotides per second, while the elongation rate for E. coli RNAP has been measured at 40-50 nucleotides per second (Sastry (1997) J Biol Chem 272:8644-52). The T7 family of RNA polymerases is structurally distinct from the multi-subunit family of RNA polymerases (including bacterial and eukaryotic sub-families). In contrast to bacterial RNA polymerases, T7 polymerase is not inhibited by the antibiotic rifampicin. Nevertheless, many common functional features are shared with these more complex enzymes.

Bacteriophage T7 RNA Polymerase is a DNA-dependent RNA polymerase that is highly specific for the T7 phage promoters. The 99 kD enzyme catalyzes in vitro RNA synthesis from a cloned DNA sequence under the T7 promoters. T7 polymerase is extremely promoter-specific and transcribes only DNA downstream of a T7 promoter (TAATACGACTCACTATAG (SEQ ID NO: 27), transcription beginning with the 3′ G). The T7 RNA polymerase also requires a double stranded DNA template and Mg²⁺ ion as cofactor for the synthesis of RNA. It has a very low error rate. The three-dimensional structure of T7 RNA polymerase shows high α-helicity with a deep cleft (Sastry (1996) Biochemistry 35:13519-30). In vitro, T7 RNA polymerase transcribes DNA without additional protein factors.

In some embodiments, the heterologous polymerase is an RNA polymerase of bacterial origin. In other embodiments, the RNA polymerase is of archeal origin.

In some embodiments, the heterologous polymerase used in the genetic circuits described herein is of eukaryotic origin. In certain embodiments, the heterologous polymerase is a RNA polymerase I, RNA polymerase II, RNA polymerase III, RNA polymerase IV, RNA polymerase V.

In certain embodiments, the genetic circuits described herein comprise any of the heterologous polymerases (e.g., RNA polymerases of viral, bacterial, eukaryotic, archeal origin, etc.) described herein and a suitable promoter/operator controlling the transcription of one or more output sequences (e.g., first, second, third, etc.), wherein the heterologous polymerase induces the transcription of the one or more output sequences (e.g., first, second, third, etc.) without the use of the native cellular transcriptional machinery.

In certain embodiments, the genetic circuits described herein comprise any of the heterologous polymerases (e.g., RNA polymerases of viral, bacterial, eukaryotic, archeal origin, etc.) described herein, one or more endogenous RNA polymerases part of the native cellular transcriptional machinery, and a suitable promoter/operator to control the transcription of one or more output sequences (e.g., first, second, third, etc.)

Several other polymerases known to those of ordinary skill in the art are also contemplated herein.

Nuclease

The term “nuclease,” as used herein, refers to an agent, for example, a protein, capable of cleaving a phosphodiester bond connecting two nucleotide residues in a nucleic acid molecule. A nuclease may be an endonuclease, cleaving a phosphodiester bond within a polynucleotide chain. Some nucleases, such as endonucleases, are site-specific nucleases, binding and/or cleaving a specific phosphodiester bond within a specific nucleotide sequence. The endonuclease described herein is a catalytically inactive endonuclease (e.g., lacks endonuclease activity) but retains the ability to bind to a specific nucleotide sequence. The terms “recognition sequence,” “recognition site,” “nuclease target site,” or “target site” are used herein to refer to a location within a nucleic acid sequence where a nuclease interacts with a specific nucleotide sequence.

In some embodiments, a nuclease is an RNA-guided (i.e., RNA-programmable) endonuclease, which is associated with (e.g., binds to) an RNA (e.g., a “guide RNA” or “gRNA,”) having a sequence that complements a recognition site, thereby providing sequence specificity to the endonuclease.

In the context of RNA-guided nucleases, a recognition site typically comprises a nucleotide sequence that is complementary to the guide RNA(s) of the RNA-guided endonuclease, and a protospacer adjacent motif (PAM) at the 3′ end adjacent to the guide RNA-complementary sequence(s). In some embodiments, a recognition site can encompass the particular sequences to which the catalytically inactive endonuclease (e.g., dCas9, dCpf1, etc.) binds.

For the RNA-guided endonuclease dCpf1 (or fusion proteins containing the guide RNA-binding domain thereof), the recognition site may be, in some embodiments, 17-25 base pairs in length plus an additional PAM sequence. PAM sequences vary in length. In some embodiments, the PAM has a length of 3-7 base pairs. In some embodiments, the PAM has a length of 3 base pairs (e.g., NNN, wherein N independently represents any nucleotide). In some embodiments (e.g., where the RNA guided nuclease has a Cpf1 binding domain), the last nucleotide of a 3 base pair PAM can be any nucleotide, while the other two nucleotides can be either C or T, but preferably T (e.g., TTN). Additional PAM sequences for Cpf1 include, but are not limited to TTTN and TTTN. In other embodiments (e.g., where the RNA-guided nuclease has a Cas1, Cas3, Cas4, Cas7, Cas9, or Cas10 binding domain), the nucleotide sequence of the PAM depends on the specific Cas protein and its species of origin (e.g., Exemplary PAM sequences for Cas9 include NRG, NGG, NGCG, NGAG, NGAG, NGG, NGG, NNGRRT, NNGRRT, NNARAA, etc.). Additional PAM sequences are exemplified in Table 3 from Braff et al. ((2016) Cold Spring Harb Protoc., the contents of which are entirely incorporated by reference).

TABLE 3
Characterized PAMs for Cas9 orthologs (Braff
et al. (2016) Cold Spring Harb Protoc.)

| Cas9 System | PAM | References | Other Notes |
|---|---|---|---|
| Streptococcus | NNAGAAW | Horvath et al. 2008; | NNAAAAW cleaved |
| thermophilus CRISPR1 | | Esvelt et al. 2013 | more efficiently |
| | | | (Fonfara et al. 2013) |
| Streptococcus | NGGNG | Horvath et al. 2008 | |
| thermophilus CRISPR3 | | | |
| Streptococcus pyogenes | NGG | Mojica et al. 2009 | |
| Streptococcus agalactiae | NGG | Mojica et al. 2009 | |
| Listeria monocytogenes | NGG | Mojica et al. 2009 | |
| Streptococcus mutans | NGG | Van der Ploeg 2009 | |
| Neisseria meningitidis | NNNNGATT | Zhang et al. 2013; | |
| | | Esvelt et al. 2013 | |
| Campylobacter jejuni | NNNNACA | Fonfara et al. 2013 | |
| Francisella novicida | NG | Fonfara et al. 2013 | |
| Streptococcus | NNGYAAA | Chen et al. 2014 | NNNGYAAA seems |
| thermophilus LMG18311 | | | to also work |
| Treponema denticola | NAAAAN | Esvelt et al. 2013 | |

Several other PAM sequences are known to one of ordinary skill in the art.

CRISPR

The Clustered Regularly Interspaced Short Palindromic Repeats (CRISPR)/endonuclease system was initially discovered in bacterial and archaeal species as a defense mechanism against foreign genetic material (e.g., plasmids and bacteriophages). For instance, the naturally occurring CRISPR/Cas systems rely on expression of three components: a guide RNA sequence that is complementary to a target sequence, scaffold RNA that aids in recruiting the third component, an endonuclease, to the site. Though in many bacterial and archaeal species CRISPR/Cas systems are used to degrade foreign genetic material, the system has been adapted for use in a wide variety of prokaryotic and eukaryotic organisms and have been used for many methods including gene knockout, mutagenesis, and expression activation or repression (Hsu, et al. (2014) Cell 157(6):1262-78).

The genetic circuit contemplated herein comprises one or more CRISPR components, such as a catalytically inactive endonuclease. In some embodiments, the catalytically inactive endonuclease is a CRISPR-associated protein (Cas) nuclease. Examples of the catalytically inactive Cas endonuclease include, but are not limited to, Cas1, Cas3, Cas4, Cas7, Cas9, or Cas10. In some embodiments, the catalytically inactive endonuclease (e.g., lacks endonuclease activity) is a catalytically inactive Cas9 “CRISPR/dCas9”. The terms “CRISPR/dCas9,” “dCas9,” “dCas9 nuclease,” or “dCas9 endonuclease” are used interchangeably to refer to an RNA-guided catalytically inactive endonuclease comprising a dCas9 protein, or a fragment thereof (e.g., a protein comprising an inactive DNA cleavage domain of Cas9).

Wildtype Cas9 is an RNA-guided DNA endonuclease associated with the CRISPR type II adaptive immunity system in Streptococcus pyogenes, among other bacteria. S. pyogenes utilizes Cas9 to interrogate and cleave foreign DNA, such as invading bacteriophage DNA or plasmid DNA (Heler et al. (2015) Nature 519(7542):199-202; Jinek et al. (2012) Science 337(6096):816-21). Native Cas9 assists in all three CRISPR steps: it participates in adaptation, participates in crRNA processing and it cleaves the target DNA assisted by crRNA and an additional RNA called tracrRNA. Native Cas9 requires a guide RNA composed of two disparate RNAs that associate to make the guide—the CRISPR RNA (crRNA), and the trans-activating RNA (tracrRNA) (Jinek et al. (2012) Science 337(6096):816-21; Rath (2015) Biochimie 117:119-28).

Cas9 orthologs have been described in various species, including, but not limited to, Streptococcus pyogenes (SpCas9), Streptococcus thermophilus (StCas9), Neisseria meningitides (NmCas9), Staphylococcus aureus (SaCas9), or Treponema denticola (TdCas9).

The catalytically inactive endonuclease described herein (e.g., dCas9) does not cleave the target dsDNA. Instead, the guide RNA guides the catalytically inactive endonuclease (e.g., dCas9) to a specific locus (e.g., promoter/operator) and blocks transcription of the downstream nucleotide sequence (e.g., output sequence). Examples of dCas9 contemplated herein include, but are not limited to, catalytically inactive variants of any of the Cas9 orthologues (e.g., dSpCas9, dspCas9^{AA}, dSpCas9^{E}, dSpCas9^{EQR}, dSpCas9^{VQR}, dSpCas9^{VRER}, desSpCas9, dSpCas9-HF1, dSaCas9, desSaCas9, dSt1Cas9, etc.), including variants or fusion proteins thereof, or other suitable dCas9 endonucleases that are catalytically inactive and sequences that are apparent to those of ordinary skill in the art.

In some embodiments, the term “dCas9” includes dCas9 variants which are at least about 70% identical, at least about 80% identical, at least about 90% identical, at least about 95% identical, at least about 98% identical, at least about 99% identical, at least about 99.5% identical, or at least about 99.9% to the native amino acid sequence of a Cas9 protein.

In some embodiments, the term “dCas9” includes dCas9 variants which are shorter or longer than the native amino acid sequence of a Cas9 protein by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids or more.

Alternatively or in addition, the endonuclease described in the present disclosure is a catalytically inactive CRISPR from Prevotella and Francisella 1 “CRISPR/dCpf1.” The terms “CRISPR/dCpf1,” “dCpf1,” “dCpf1 nuclease,” or “dCpf1 endonuclease” are used interchangeably to refer to an RNA-guided catalytically inactive endonuclease comprising a dCpf1 protein, or a fragment thereof (e.g., a protein comprising an inactive DNA cleavage domain of Cpf1) (see, e.g., Zetsche et al. (2015) Cell 163, 759-71, the entire contents of which are incorporated herein by reference).

Wildtype Cpf1 cleaves double stranded DNA (dsDNA) when targeted to a specific locus with a complementary guide RNACpf1-containing CRISPR systems have at least three unique features: (1) Cpf1-associated CRISPR arrays are processed into crRNAs without the requirement of a trans-acting crRNA; (2) Cpf1-crRNA complexes cleave target DNA proceeded by a short T-rich protospacer-adjacent motif (PAM); and (3) DNA cleavage by Cpf1 generates a double strand break with a 4-5 nucleotide 5′ overhang (Zetsche et al. (2015) Cell 163, 759-71).

Cpf1 orthologs have been described in various species, including, but not limited to, Parcubacteria bacterium GWC2011_GWC2_44_17 (PbCpf1), Lachnospiraceae bacterium ND2006 (LbCpf1), Butyrivibrio proteoclasticus (BpCpf1), Peregrinibacteria bacterium GW2011_GWA_33_10 (PeCpf1), Acidaminococcus sp. BV3L6 (AsCPF1), Porphyromonas macacae (PmCpf1), Lachnospiraceae bacterium ND2006 (LbCpf1), Porphyromonas crevioricanis (PcCpf1), Prevotella disiens (PdCpf1), Moraxella bovoculis 237 (MbCpf1), Smithella sp. SC_K08D17 (SsCpf1), Leptospira inadai (LiCpf1), Lachnospiraceae bacterium MA2020 (Lb2Cpf1), Francisella novicida (FnCpf1), Candidatus Methanoplasma termitum (CMtCpf1), and Eubacterium eligens (EeCpf1) (see, e.g., Zetsche et al. (2015) Cell 163, 759-71).

The catalytically inactive endonuclease described herein (e.g., dCpf1) does not cleave the target dsDNA. Instead, the guide RNA guides the catalytically inactive endonuclease to a specific locus (e.g., promoter/operator) and blocks transcription of the downstream nucleotide sequence (e.g., output sequence). Examples dCpf1 contemplated herein include, but are not limited to, catalytically inactive variants of any of the Cpf1 orthologs described herein (e.g., dAsCpf1, dFnCpf1, dLbCpf1, etc.), including variants or fusion proteins thereof, or other suitable dCpf1 endonucleases that are catalytically inactive and sequences that are apparent to those of ordinary skill in the art.

In some embodiments, the term “dCpf1” includes dCpf1 variants which are at least about 70% identical, at least about 80% identical, at least about 90% identical, at least about 95% identical, at least about 98% identical, at least about 99% identical, at least about 99.5% identical, or at least about 99.9% to the native amino acid sequence of a Cpf1 protein. In some embodiments, the nucleotide sequence encoding the dCpf1 nuclease may be codon optimized for expression in a host cell or organism.

In some embodiments, the dCpf1 variants are shorter or longer than the native amino acid sequence of a Cpf1 protein by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids or more. Methods for cloning, generating, and purifying a dCas9 and/or a dCpf1 sequence/protein (or a fragment thereof) are known and apparent to those of skill in the art (see, e.g., Zetsche et al. (2015) Cell 163, 759-71).

Guide RNA

In genetically engineered CRISPR systems, the requirement for three independent components can be circumvented by expression of a small guide RNA (sgRNA) that contains both the CRISPR guide RNA sequence for binding a target sequence and the scaffold RNA that together mimics the structure formed by the individual guide RNA sequence and scaffold sequence and is sufficient to recruit the endonuclease to the appropriate target site (Jinek, et al. (2012) Science 337(6096):816-21).

The terms “guide RNA,” “gRNA,” and “CRISPR guide sequence” may be used interchangeably throughout and refer to a nucleic acid comprising a sequence that determines the specificity of an endonuclease, such as a catalytically inactive endonuclease binding protein of a CRISPR system (e.g., dCas9, dCpf1, etc.). The guide RNA sequence targets the catalytically inactive endonuclease (e.g., dCas9, dCpf1, etc.) to a target nucleic acid sequence, also referred to as a “target site.” For clarity and ease of explanation, a guide RNA may be referred to as first, second, third or fourth guide RNA (and so on) so as to distinguish one guide RNA from another guide RNA.

The term “RNA-guided endonuclease” refers to a nuclease that complexes with (e.g., binds or associates with) one or more RNA(s) that is not a target for cleavage. Generally, the bound RNA is referred to as a “guide RNA.” Guide RNAs can exist as a complex of two or more RNAs, or as a single RNA molecule. The guide RNA comprises a nucleotide sequence that complements a recognition site, which mediates binding of the nuclease/RNA complex to the recognition site, providing the sequence specificity of the nuclease:RNA complex. Typically, guide RNAs that exist as single RNA species comprise two domains: (1) a “guide” domain that shares homology to a target nucleic acid (e.g., and directs binding of a dCpf1 complex to the target); and (2) a “direct repeat” domain that binds an RNA-guided endonuclease. In this way, the sequence and length of the guide RNA may vary depending on the specific recognition site sought and/or the specific RNA-guided endonuclease utilized (see e.g., Zetsche et al. (2015) Cell 163, 759-71). Indeed, all RNA guided endonucleases are able to bind guide RNAs of various sequences. Because RNA-guided endonucleases use RNA:DNA hybridization to determine target DNA cleavage sites, these proteins are able to bind to and cleave any sequence specified by the guide RNA. In the case of catalytically inactive RNA-guided endonucleases, such as dCas9 and dCpf1, the proteins bind to the sequence specified by the guide RNA, but do not cleave the sequence. In some embodiments, the direct repeat domain may be 16-22 base pairs in length. In some embodiments, the entire length of the guide RNA is 33-47 base pairs in length.

The guide RNAs may be produced by any method or obtained from any source known to one of ordinary skill in the art. For example, the guide RNA sequence may be any nucleic acid sequence of the indicated length present in the nucleic acid of a host cell (e.g., genomic nucleic acid and/or extra-genomic nucleic acid). In some embodiments, guide RNA sequences may be designed and synthesized to target desired nucleic acids (e.g., nucleic acids encoding transcription factors, signaling proteins, transporters, etc.).

In some embodiments, the guide RNAs of the present disclosure have a length of 10 to 500 nucleotides. In some embodiments, a guide RNA has a length of 10 to 20 nucleotides, 10 to 30 nucleotides, 10 to 40 nucleotides, 10 to 50 nucleotides, 10 to 60 nucleotides, 10 to 70 nucleotides, 10 to 80 nucleotides, 10 to 90 nucleotides, 10 to 100 nucleotides, 20 to 30 nucleotides, 20 to 40 nucleotides, 20 to 50 nucleotides, 20 to 60 nucleotides, 20 to 70 nucleotides, 20 to 80 nucleotides, 20 to 90 nucleotides, 20 to 100 nucleotides, 30 to 40 nucleotides, 30 to 50 nucleotides, 30 to 60 nucleotides, 30 to 70 nucleotides, 30 to 80 nucleotides, 30 to 90 nucleotides, 30 to 100 nucleotides, 40 to 50 nucleotides, 40 to 60 nucleotides, 40 to 70 nucleotides, 40 to 80 nucleotides, 40 to 90 nucleotides, 40 to 100 nucleotides, 50 to 60 nucleotides, 50 to 70 nucleotides, 50 to 80 nucleotides, 50 to 90 nucleotides or 50 to 100 nucleotides. In some embodiments, a guide RNA has a length of 10 to 200 nucleotides, 10 to 250 nucleotides, 10 to 300 nucleotides, 10 to 350 nucleotides, 10 to 400 nucleotides or 10 to 450 nucleotides. In some embodiments, the guide RNA has a length of 17 to 25 nucleotides. In some embodiments, a guide RNA has a length of more than 500 nucleotides. In some embodiments, a guide RNA has a length of 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500 or more nucleotides.

It will be appreciated that a guide RNA sequence, or portion thereof, is complementary to a target nucleic acid in a host cell if the guide RNA sequence is capable of hybridizing to the target nucleic acid. In some embodiments, the guide RNA sequence is at least 50%, 55%, 60%, 65%, 70%, 75%, 80%, 85%, 90%, 95%, 96%, 97%, 98%, 99%, or at least 100% complementary to a target nucleic acid (see also U.S. Pat. No. 8,697,359, which is incorporated by reference for its teaching of complementarity of a guide RNA sequence with a target polynucleotide sequence).

It has been demonstrated that mismatches between a guide RNA and the target nucleic acid near the 3′ end of the target nucleic acid may abolish nuclease cleavage activity (Upadhyay et al. (2013) G3 3(12):2233-8). In some embodiments, the CRISPR guide sequence is at least 50%, 55%, 60%, 65%, 70%, 75%, 80%, 85%, 90%, 95%, 96%, 97%, 98%, 99%, or at least 100% complementary to the 3′ end of the target nucleic acid (e.g., the last 5, 6, 7, 8, 9, or 10 nucleotides of the 3′ end of the target nucleic acid).

Output

As described herein, an “output sequence” refers to any sequence or resulting molecule under the control of (e.g., produced in response to) an input signal. In some embodiments, the one or more output sequences are non-coding RNA (e.g., rRNAs, tRNAs, small nuclear RNAs (snRNAs), small nucleolar RNAs (snoRNAs), miRNAs, guide RNAs, small RNAs that sequester the guide RNA, etc.). In some embodiments, the one or more output sequences are coding RNAs that encode one or more output molecules (e.g., 2 or more different proteins, such as fluorescent proteins (e.g., GFP, RFP, etc.) and/or therapeutic proteins). As described herein, an “output molecule” refers to the molecule transcribed from an output sequence, when the output sequence is a coding RNA. In some embodiments, one or more of the output molecule is one or more therapeutic proteins. As described herein, the term “therapeutic protein” refers to a protein used in the treatment of a subject having a disorder, pathological condition or disease.

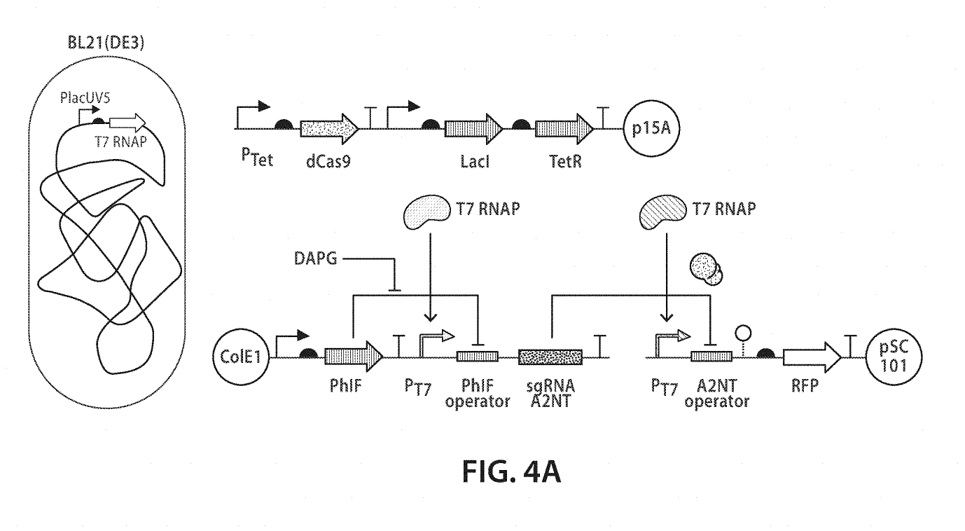

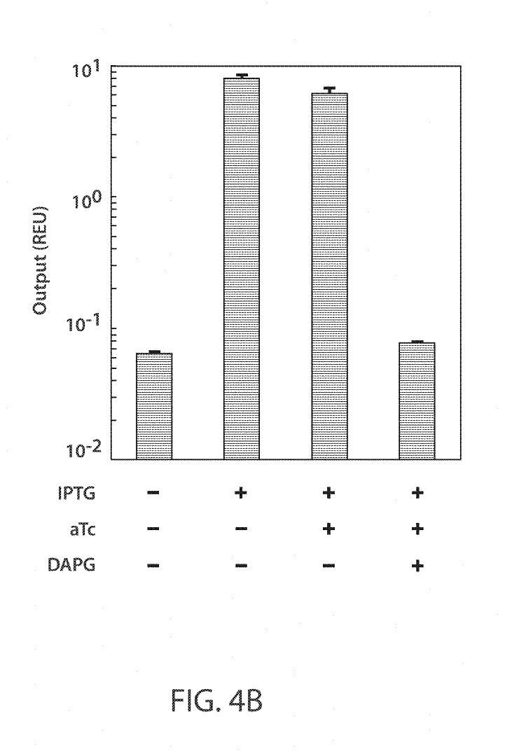

Genetic circuits of the present disclosure, in some embodiments, generate a response in the form of an output sequence. For example, as shown in FIG. 4A, RFP is produced from transcription of an output sequence produced in response to inhibition of the Tet promoter (TetP) by IPTG. The genetic circuits described herein may contain one or multiple (e.g., 2, 3, 4 or more) copies of an output sequence. In some embodiments, a genetic circuit contains two or more copies of the same output sequence. In some embodiments, a genetic circuit contains two or more (e.g., 2, 3, 4, 5, 6, 7, 8, 9, 10 or more) different output sequences. In some embodiments, the two or more different output sequences are non-coding RNA (e.g., rRNAs, tRNAs, small nuclear RNAs (snRNAs), small nucleolar RNAs (snoRNAs), miRNAs, guide RNAs. small RNAs that sequester the guide RNA, etc.). In some embodiments, the two or more output sequences are coding RNAs that encode two or more output molecules (e.g., 2 or more different proteins, such as fluorescent proteins (e.g., GFP, RFP, etc.) and/or therapeutic proteins (e.g., antibodies, immunomodulatory peptides, etc.)).

In some embodiments, a first output sequence controls transcription of a second output sequence that encodes an output molecule (e.g., a first guide RNA binds to and guides dCas9 or dCpf1 to bind to a promoter/operator sequence to reduce or block transcription of a mRNA that encodes red fluorescent protein (RFP). For example, with reference to FIGS. 4A-4B, IPTG activates the lac operon, which in turn induces the transcription of the Tet repressor (TetR). TetR subsequently binds to the Tet promoter and decreases the expression of a catalytically-inactive endonuclease (e.g., dCas9, dCpf1, etc.) which binds to a nucleotide sequence specified by the guide RNA. Thus, IPTG is considered an input signal that modulates the Tet promoter and, in turn, expression of one or more output sequence(s), such as RFP. In this example, due to reduced expression of the a catalytically-inactive endonuclease, the expression of RFP is increased. A similar effect is observed in the absence of a second input signal, DAPG, which controls expression of the guide RNA that targets the catalytically-inactive endonuclease to the operator upstream of the RFP-encoding sequence. Likewise, a guide RNA can also be considered an input signal. In some embodiments, the guide RNA binds to and guides the catalytically inactive endonuclease (e.g., dCas9, dCpf1, etc.) to a specific sequence in a target promoter/operator and reduces, or in some embodiments, blocks downstream transcription of one or more output sequence(s).

For clarity and ease of explanation, output sequences and/or output molecules may be referred to as a first, second, third or fourth output sequence and/or output molecules (and so on) so as to distinguish one output sequence and/or output molecule from another. In some embodiments, reference to a first output sequence and/or output molecule and a second output sequence and/or output molecule, encompasses two different output sequences and/or output molecules (e.g., first guide RNA v. second guide RNA; first protein v. second protein). In some embodiments, the first and second output sequences and/or output molecules may be the same, for example, in order to provide an extra copy of a first guide RNA for the purpose of increased expression of the first guide RNA.

Genetic Circuits

The genetic circuits of the present disclosure may be controlled by input signals to generate one or more output sequences and/or output molecules in a cell. Thus, in some embodiments, a genetic circuit comprises (a) a first promoter/operator regulated by a catalytically inactive endonuclease and guide RNA controlling transcription of an output sequence; (b) a second promoter/operator responsive to a first input signal that encodes a guide RNA; and, (c) a third promoter/operator responsive to a second input signal that encodes the catalytically inactive endonuclease.

In some embodiments, the genetic circuit further comprises: (d) a fourth promoter/operator responsive to a third input signal that controls the transcription of one or more output sequences and/or output molecules.

In some embodiments, the genetic circuit further comprises: (e) a fifth promoter/operator responsive to a fourth input signal that controls the transcription of one or more output sequences and/or output molecules.

Tuning and control of a genetic circuit may also be achieved, for example, by controlling the level of nucleic acid expression of particular components of the circuit. This control can be achieved, for example, by controlling copy number of the nucleic acids (e.g., using low, medium and/or high copy plasmids, and/or constitutively-active promoters).

The temporal and sequential activation of the one or more components of the genetic circuit may be evaluated by assessing the levels of one or more output sequence(s) and/or one or more output molecule(s). In some aspects, the method comprises detecting an expression level of the output sequence and/or output molecule and, optionally, quantifying levels of the output sequence and/or output molecule. In some embodiments, the output of the promoter/operator(s) involves assessing the expression or activity of the gene promoter/operator(s). Methods of assessing expression of one or more components of the genetic circuit described herein will be evident to one of skill in the art and includes, for example, qRT-PCR, microarray analysis, Northern blotting, and RNA-Seq. In some embodiments, the output sequence is a gene, guide RNA, etc. and expression of the output sequence is evaluated by quantifying the amount of the product of the output sequence (e.g., protein levels). In some embodiments, the product of the output sequence is a protein. Methods of assessing protein expression of one or more components of the genetic circuit described herein will be evident to one of skill in the art and includes, for example, Western blotting, enzyme-linked immunosorbent assay (ELISA), cell-Based ELISA, intracellular flow cytometry, immunocytochemistry, mass spectrometry, activity assays, etc.

Non-Linearity of Genetic Circuits

The genetic circuits provided herein can be implemented by connecting a sensory input signal to the transcription of a guide RNA, and using the guide RNA to target the catalytically inactive endonuclease to a specific output gene to cause transcriptional repression. As used herein, the term “gate” refers to an input-output unit. Guide RNA gates are normally non-cooperative, eliciting linear input-output response curves. Described herein are several strategies to introduce non-linearity into the otherwise linear response of a gate in a genetic circuit.

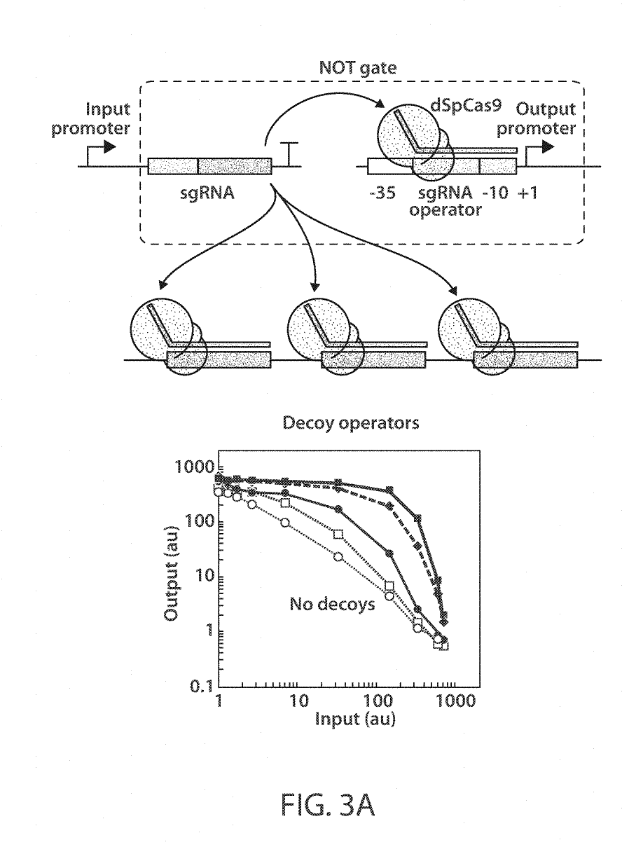

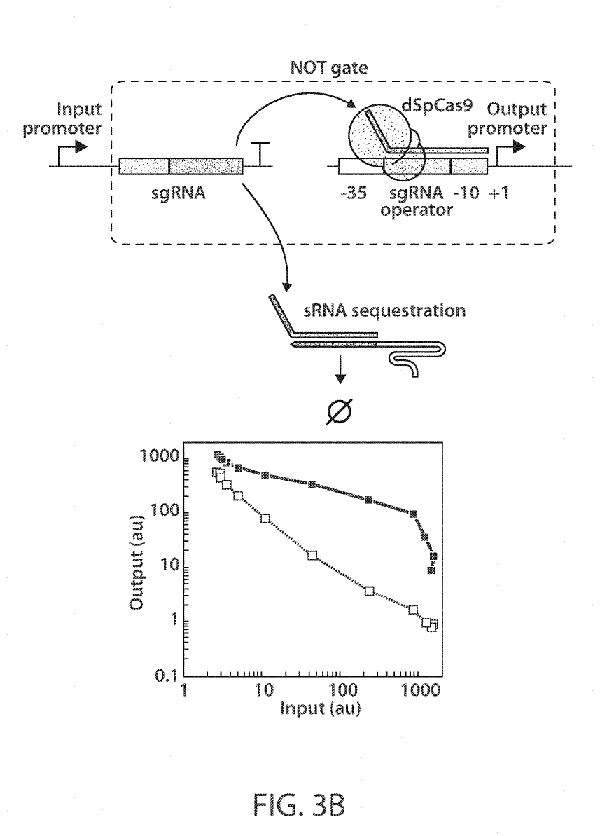

In some embodiments, non-linearity can be introduced using a decoy DNA site or small RNA (sRNA). A decoy DNA site attracts a guide RNA and catalytically inactive endonuclease to an off-target site and reduces the interaction of the guide RNA and catalytically inactive endonuclease with a target promoter/operator sequence (e.g., first, second, third, fourth, fifth promoter/operators, etc.). In some embodiments, non-linearity can be introduced using sRNAs. sRNAs introduce non-linearity by degrading guide RNAs, as shown for example in FIG. 3B.

Other examples of ways to introduce non-linearity and tune the response function of a guide RNA provided herein include, without limitation, the use and/or manipulation of ribozyme processing, guide RNA operon, guide mutation, operator location, termination heterologous (e.g., T7)-based circuit, promoter looping, multiple promoter/operators, and/or split guide RNAs, as described below.

Ribozyme Processing:

Ribozymes known in the art (e.g., hammerhead, HDV ribozymes, etc.) are genetically fused upstream or downstream from a guide RNA to catalytically cleave the RNA once the one or more ribozymes is transcribed, resulting in a guide RNA with fewer non-essential flanking RNA sequences. In some embodiments, the one or more ribozymes remove an upstream RNA region contributed from a promoter region, a downstream terminator region, etc.

Guide RNA Operon:

Multiple guide RNAs within a guide RNA operon allow multiple targeting guide sequences to be transcribed from a single promoter, which increases the resulting amount of downstream gene regulation. In some embodiments, the multiple guide RNAs in an operon each target different regions, in which case a single transcriptional input results in “fan-out” by regulating multiple output sequences. In some embodiments, the multiple guide RNA regions in an operon may all target the same locus, in which case the strength of gene regulation is increased for the same amount of transcriptional input. In some embodiments, the guide RNAs are covalently connected. In some embodiments, the guide RNAs are cleaved into multiple non-covalently linked guide RNAs.

Guide Mutation:

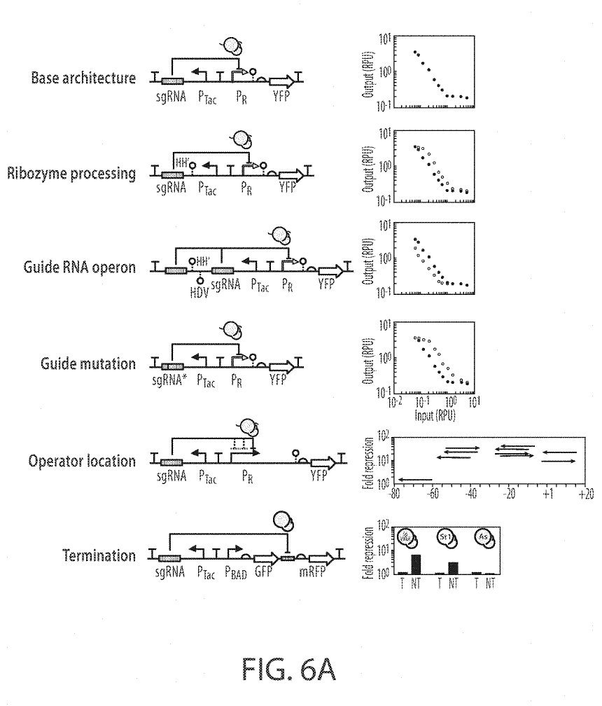

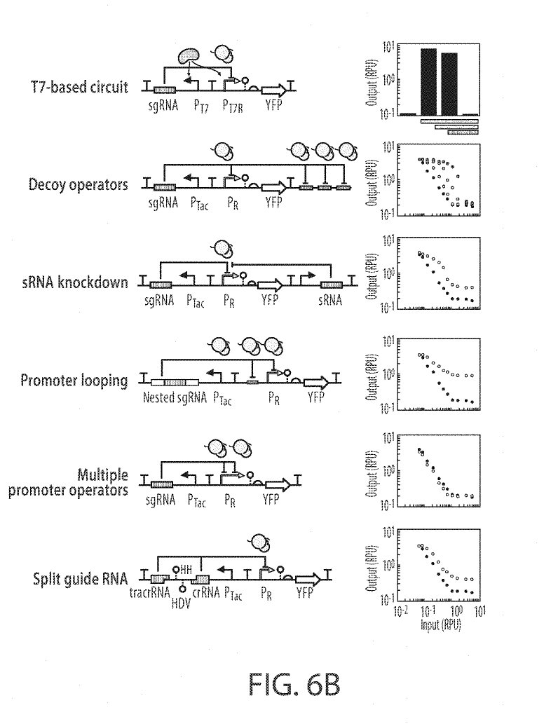

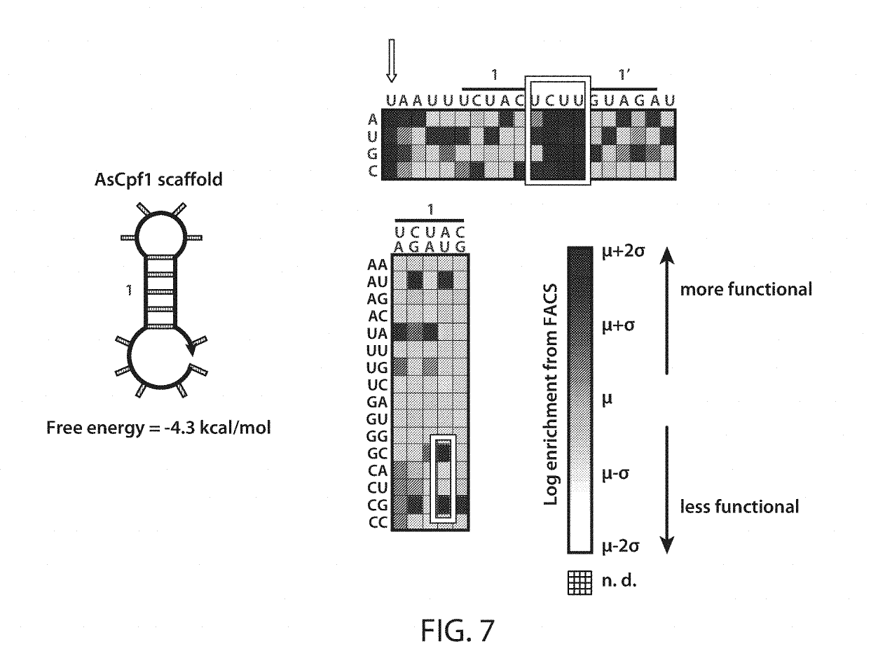

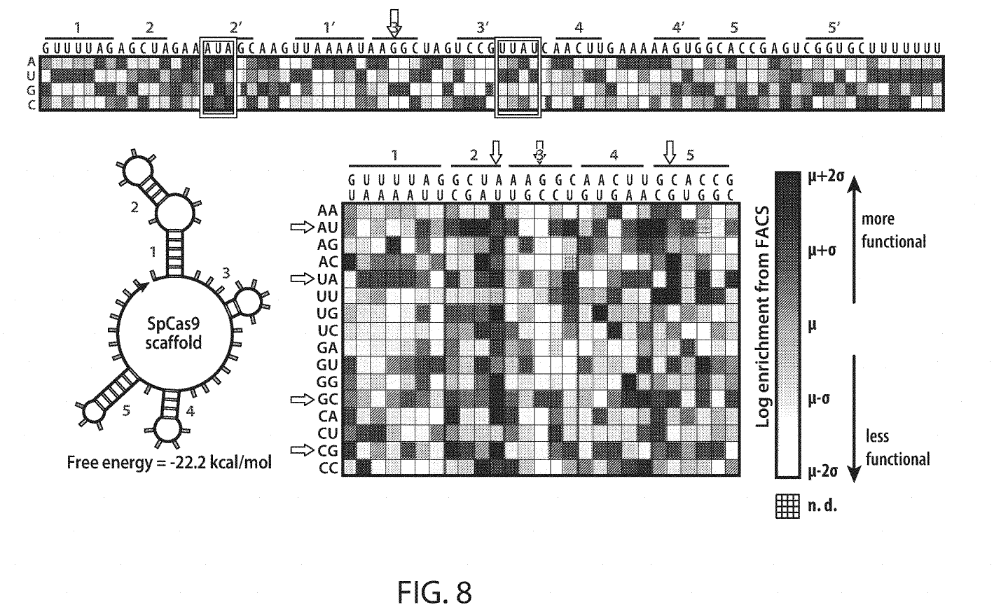

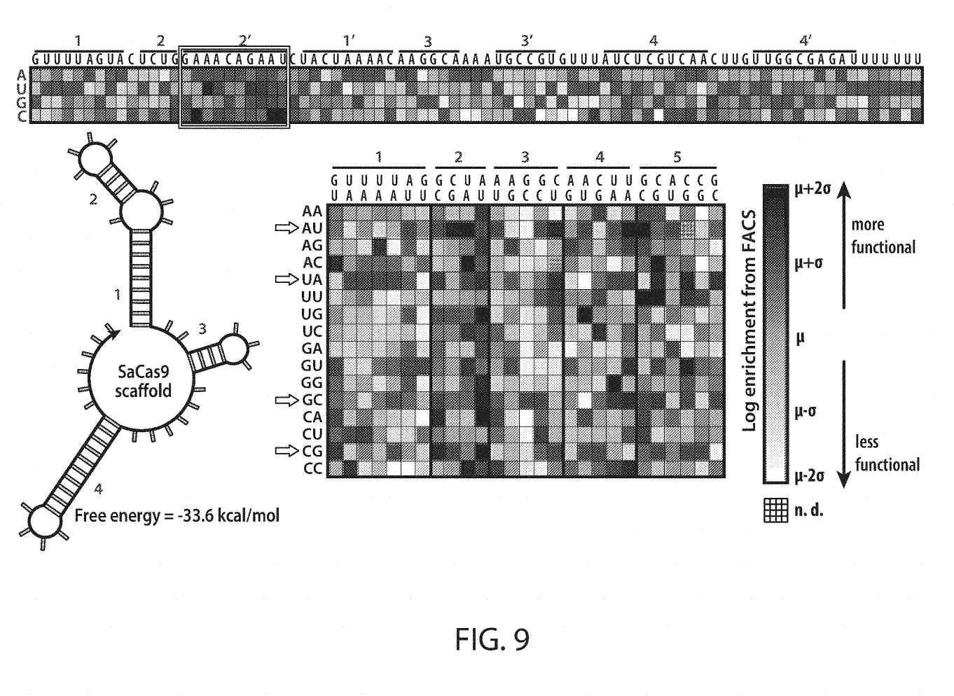

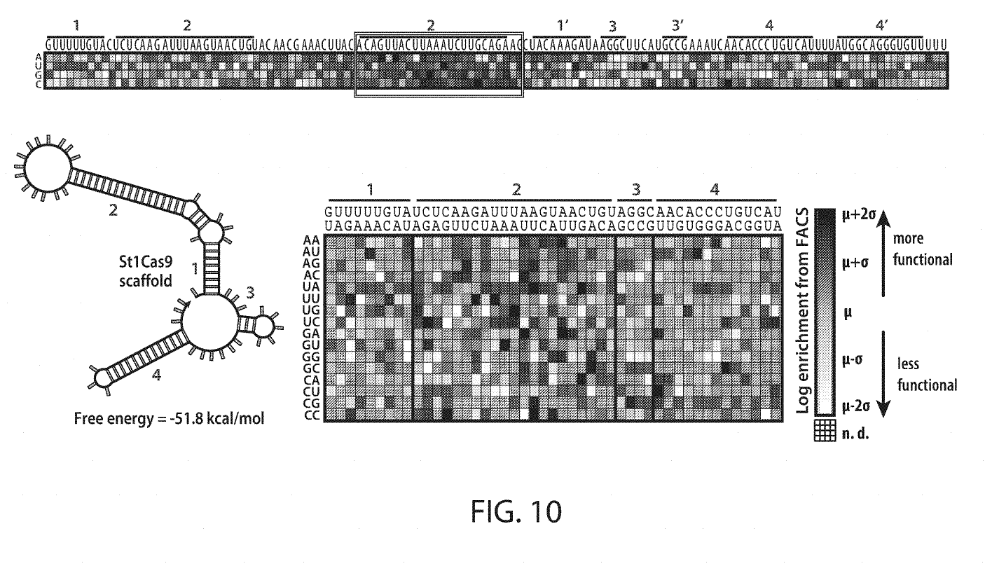

In some embodiments, mutations in guide RNAs, or changes to their length, is used to change the dissociation constant of the guide RNA-inactive endonuclease-DNA complex in order to modulate the characteristics of the guide RNA response function. Non-limiting examples include the use of a guide RNA that is shorter or has one or more mismatches with the target locus results in a greater dissociation constant, and shifts the response function “to the right” (FIG. 6A).

Operator Location:

In some embodiments, the position of the small guide RNA (sgRNA)-targeted operator sequence is moved to increase or decrease the strength of the sgRNA targeting. In some embodiments, the operator is upstream in relation to the transcription start site, downstream in relation to the transcription start site, shortened, lengthened, or placed on either the sense or antisense DNA strand. In some embodiments, multiple operator sequences are positioned within the same promoter to achieve cooperativity.

Termination:

In some embodiments, guide RNA-inactive endonuclease targeting is used to regulate not only transcription initiation, but also transcription termination. In some embodiments, the “operator” is placed downstream from a promoter, either within or between genes, resulting in transcriptional termination. In other embodiments, quantitatively different levels of transcription termination are engineered using a different DNA strand for the operator, different guide RNA mutants, or different inactive endonuclease mutants.

Heterologous-Based Circuit:

In some embodiments, a heterologous synthetic circuit or transcription machinery is used to create a transcription machinery that is independent from the host transcription system. In some embodiments, T7 RNA polymerase is used as a heterologous transcription machinery. Heterologous synthetic circuits, such as the T7-based circuits can operate in a “synthetic layer” without the regulation that goes into natural transcriptional processes. In addition, the heterologous synthetic circuits, such as the T7 circuits, are portable and can be transferred between organisms with relative ease so that promoter sequences do not have to be re-engineered to interface with the endogenous host polymerase.

Split Guide RNA:

Split guide RNAs physically separate regions of the guide RNA into multiple fragments, and tether each fragment to an RNA association-domain. In some embodiments, the association of these fragments is used to increase the molecularity and cooperativity of the target riboprotein inactive endonuclease complex.

Promoter Looping:

Promoter looping occurs by physically bending the DNA around the promoter region to obtain steric cooperativity. In some embodiments, promoter looping is achieved by targeting multiple guide RNA-inactive endonuclease complexes to separate regions of the promoter and having these complexes bind each other through protein affinity domains fused to the inactive endonuclease or through RNA affinity domains fused to the guide RNAs. In other embodiments, rather than using multiple guide RNAs, a single “multi-targeting” guide RNA can be introduced which contains multiple targeting regions and inactive endonuclease scaffold regions. The multi-targeting guide RNA associates with multiple inactive endonucleases in the promoter region, and the length of the multi-targeting guide RNA imposes a spatial constraint to the geometry of the bound promoter. Non-limiting examples of engineering multi-targeting guide RNAs include genetically concatenating multiple guide RNAs in one RNA transcript without intervening transcriptional terminators, making a genetic fusion by “nesting” one guide RNA region in an RNA loop in the middle of another guide RNA region, etc.

Nucleic Acid

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides).

In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, gRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides.

Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate (e.g., in the case of chemically synthesized molecules), nucleic acids can comprise nucleoside analogs such as analogs having chemically modified bases or sugars, and backbone modifications. These modification may alter a chemical property of the molecules, such as its degradation or binding kinetics. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Nucleic acids (e.g., components, or portions, of the nucleic acids) of the present disclosure may be naturally occurring or engineered. Engineered nucleic acids include recombinant nucleic acids and synthetic nucleic acids. “Recombinant nucleic acids” may refer to molecules that are constructed by joining nucleic acid molecules and, in some embodiments, can replicate in a living cell. “Synthetic nucleic acids” may refer to molecules that are chemically or by other means synthesized or amplified, including those that are chemically or otherwise modified but can base pair with naturally occurring nucleic acid molecules. Recombinant and synthetic nucleic acids also include those molecules that result from the replication of either of the foregoing.

The nucleic acids may be single-stranded (ss) or double-stranded (ds), as specified, or may contain portions of both single-stranded and double-stranded sequence. The nucleic acid may be DNA, both genomic and cDNA, RNA or a hybrid/chimeric, where the nucleic acid contains any combination of deoxyribonucleotides and ribonucleotides, and any combination of bases, including uracil, adenine, thymine, cytosine, guanine, inosine, xanthine, hypoxanthine, isocytosine, and isoguanine.

The nucleic acids of the present disclosure may be engineered using, for example, standard molecular cloning methods (see, e.g., Current Protocols in Molecular Biology, Ausubel, F. M., et al., New York: John Wiley & Sons, 2006; Molecular Cloning: A Laboratory Manual, Green, M. R. and Sambrook J., New York: Cold Spring Harbor Laboratory Press, 2012; Gibson, D. G., et al., Nature Methods 6(5):343-345 (2009), the teachings of which relating to molecular cloning are herein incorporated by reference).

Some aspects of the present disclosure provide methods and systems that use multiple components, such as gene(s) encoding catalytically inactive endonuclease and gene(s) encoding guide RNAs of a biosynthetic pathway. It should be understood that components may be encoded by a single nucleic acid (e.g., on the same plasmid or other vector) or by multiple different (e.g., independently-replicating) nucleic acids.

Some engineered nucleic acids may include nucleotide sequences homologous to a chromosomal locus of a host cell of interest. Such sequences facilitate integration of an engineered nucleic acid into a chromosomal locus of a host cell. It should be understood, however, that chromosomal integration of an engineered nucleic acid is optional.

In some embodiments, an engineered nucleic acid also comprises an antibiotic resistance gene (see, e.g., online database: card.mcmaster.ca) to facilitate cloning and selection of the nucleic acid. Thus, in some embodiments, an engineered nucleic acid comprises a kanamycin resistance gene, spectinomycin resistance gene, streptomycin resistance gene, ampicillin resistance gene, carbenicillin resistance gene, bleomycin resistance gene, erythromycin resistance gene, polymyxin B resistance gene, tetracycline resistance gene, chloramphenicol resistance gene, hygromycin resistance gene and/or a ts-resistance gene resistance gene.

Vectors

In some embodiments, one or more components of the genetic circuits described herein are provided on a recombinant expression vector. As used herein, a “vector” may be any of a number of nucleic acids into which a desired sequence or sequences may be inserted, for example, by restriction digestion and ligation or by recombination for transport between different genetic environments or for expression in a host cell. Vectors are typically composed of DNA, although RNA vectors are also available. Vectors include, but are not limited to: plasmids, fosmids, phagemids, virus genomes, and artificial chromosomes. In some embodiments, the vector is a lentiviral vector. In some embodiments, a gene encoding a catalytically inactive endonuclease, such a catalytically inactive CRISPR endonuclease (e.g., dCas9, dCpf1, etc.) is provided on a vector. In some embodiments, a gene encoding the catalytically inactive endonuclease (e.g., dCas9, dCpf1, etc.) is provided on the same vector as other one or more components (e.g., heterologous polymerase) of the genetic circuits described herein. In some embodiments, a vector comprising a gene encoding the catalytically inactive endonuclease (e.g., dCas9, dCpf1, etc.) is provided on a different vector than a vector containing one or more components (e.g., heterologous polymerase) of the genetic circuits described herein.

Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., galactosidase, fluorescence, luciferase or alkaline phosphatase), and genes which visibly affect the phenotype of transformed or transfected cells, hosts, colonies or plaques (e.g., green fluorescent protein, red fluorescent protein). Preferred vectors are those capable of autonomous replication and expression of the structural gene products present in the DNA segments to which they are operably joined.

Methods of delivering vectors are well known in the art and may include DNA, RNA, or transposon electroporation, transfection reagents such as liposomes or nanoparticles to delivery DNA, RNA, or transposons; delivery of DNA, RNA, or transposons or protein by mechanical deformation (see, e.g., Sharei et al. (2013) Proc Natl Acad Sci USA 110(6):2082-2087); or viral transduction.

In some embodiments, the vectors are administered to a cell or a subject, and thereby to the cells of the subject, by viral transduction. Exemplary viral methods for delivery include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors, and adeno-associated virus (AAV) vectors (see, e.g., PCT Publication Nos. WO 94/12649, WO 93/03769; WO 93/19191; WO 94/28938; WO 95/11984 and WO 95/00655).

In some embodiments, the vectors for expression of one or more components (e.g., catalytically inactive endonuclease, guide RNA, heterologous polymerase, etc.) of the genetic circuits described herein are retroviruses, lentiviruses or adeno-associated viruses.

In examples in which the vectors encoding one or more components of the genetic circuit are administered to a subject using a viral vector, viral particles that are capable of infecting cells of a subject and carry the vector may be produced by any method known in the art and can be found, for example in PCT Application No. WO 1991/002805A2, WO 1998/009271 A1, and U.S. Pat. No. 6,194,191. The viral particles are harvested from the cell culture supernatant and may be isolated and/or purified prior to administration of the viral particles.

A “subject” shall mean a human or a mammal including, but not limited to, a dog, cat, horse, cow, pig, sheep, goat, chicken, rodent, e.g., rats and mice, and primate, e.g., monkey. Preferred subjects are human subjects. The human subject may be a pediatric, adult or a geriatric subject.

The term “effective amount,” as used herein, refers to an amount of one or more genetic circuit components that is sufficient to elicit a desired biological response. For example, in some embodiments, an effective amount of a catalytically inactive endonuclease may refer to the amount of the endonuclease that is sufficient to reduce, or in some embodiments, block transcription of a target site (e.g., an effective amount of vector encoding dCas9 and vector encoding a guide RNA to specifically bind and reduce or block activity of the promoter/operator).

Thus, the disclosure involves, in some aspects, administering an effective amount of a genetic circuit component to produce an effective amount of one or more output molecules (e.g., therapeutic proteins, etc.) to treat the subject.

The term “treatment” or “treat” is intended to include prophylaxis, amelioration, prevention or cure of a condition. Treatment after a condition has stated aims to reduce, ameliorate or altogether eliminate the condition, and/or one or more of its associated symptoms, or prevent it from becoming worse. Treatment of subjects before a condition has started (i.e., prophylactic treatment) aims to reduce the risk of developing the condition and/or lessen its severity if the condition later develops.

As used herein, the term “prevent” refers to the prophylactic treatment of subjects who are at risk of developing a condition which treatment results in a decrease in the probability that the subject will develop the condition, or results in an increase in the probability that the condition is less severe than it would have been absent the treatment.

Treatments may reduce mortality, or extend life expectancy, of subjects having the condition as compared to subjects not treated with the genetic circuits described herein. For example, the desirable response may be inhibiting the progression of the disorder, condition or disease (e.g., autoimmune disorder, cancer, infection, aging, etc.). This may involve only slowing the progression of the disorder, condition or disease temporarily, although more preferably, it involves halting the progression of the disorder, condition or disease permanently. This can be monitored by routine diagnostic methods known to those of ordinary skill in the art.

It should be understood that, in some embodiments, the genetic circuits described herein and/or the output molecules produced by the genetic circuits described herein are used to treat or prevent the disorder, condition or disease, that is, they may be used prophylactically in subjects at risk of developing the disorder, condition or disease. Thus, an effective amount is that amount which can lower the risk of, lessen the severity of, or perhaps prevent altogether the development of the disorder, condition or disease.

The factors involved in determining an effective amount are well known to those of ordinary skill in the art and can be addressed with no more than routine experimentation. It is generally preferred that a maximum dose of the therapeutic agents (e.g., therapeutic proteins) produced by the genetic circuits disclosed herein (alone or in combination with other therapeutic agents) be used, that is, the highest safe dose according to sound medical judgment.

In some embodiments, one or more of the therapeutic agents (e.g., therapeutic proteins) expressed by the genetic circuit may be administered alone, in a pharmaceutical composition or combined with other therapeutic agent(s) or regimens. Optionally other therapeutic agent(s) may be administered simultaneously or sequentially. When the other therapeutic agent(s) are administered simultaneously they can be administered in the same or separate formulations, but are administered at the same time. The separation in time between the administration of these therapeutic agents may be a matter of minutes or it may be longer, such as 1, 2, 3, 4, 5, 6, 7, 8, 9, 10, 11 or 12 hours, or more, including 1, 2, 3, 4, 5, 6, 7 days or more.

The pharmaceutical compositions used herein are preferably sterile and contain effective amounts of the genetic circuit components and/or therapeutic agents (e.g., therapeutic proteins) produced by the genetic circuits disclosed herein for producing the desired response in a unit of weight or volume suitable for administration to a subject. The doses of pharmacological agent(s) administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits. The dosage of a pharmacological agent may be adjusted by the individual physician or veterinarian, particularly in the event of any complication.

A therapeutically effective amount typically varies from 0.01 mg/kg to about 1000 mg/kg, preferably from about 0.1 mg/kg to about 200 mg/kg, and most preferably from about 0.2 mg/kg to about 20 mg/kg, in one or more dose administrations daily, for one or more days.

Homology

The terms “homology” or “homologous,” as used herein are understood in the art to refer to nucleic acids or polypeptides that are highly related at the level of nucleotide and/or amino acid sequence. Nucleic acids or polypeptides that are homologous to each other are termed “homologues.” Homology between two sequences can be determined by sequence alignment methods known to those of ordinary skill in the art. In accordance with the present disclosure, two sequences are considered to be homologous if they are at least about 50-60% identical, e.g., share identical residues (e.g., amino acid residues) in at least about 50-60% of all residues comprised in one or the other sequence, at least about 70% identical, at least about 80% identical, at least about 90% identical, at least about 95% identical, at least about 98% identical, at least about 99% identical, at least about 99.5% identical, or at least about 99.9% identical, for at least one stretch of at least 20, at least 30, at least 40, at least 50, at least 60, at least 70, at least 80, at least 90, at least 100, at least 120, at least 150, or at least 200 amino acids. Also, as used herein, the term “substantially the same” refers to a polynucleotide sequence at least about 90% identical, at least about 95% identical, at least about 98% identical, at least about 99% identical, at least about 99.5% identical, or at least about 99.9% identical to another polynucleotide sequence.

Cell

Also provided herein are recombinant cells comprising any of the genetic constructs described herein. In some embodiments, one or more components of the genetic circuits described herein are expressed in a cell. In some embodiments, the one or more components of the genetic circuit (e.g., the nucleotide sequence encoding the catalytically inactive endonuclease, for example, dCas9 or dCpf1) may be codon optimized for expression in a cell or organism.

In certain embodiments, the cell or recombinant cell may be a prokaryotic cell. In certain embodiments, the cell is a eukaryotic cell. Examples of cells include, without limitation, bacterial cells, algal cells, plant cells, insect cells, fungal cells, yeast cells, mammalian cells, non-human mammalian cells, and human cells. In some embodiments, the cell is a bacterial cell In some embodiments, the cell is a plant cell. In some embodiments, the cell is a human cell.

In other embodiments, the cell is a fungal cell such as a yeast cell, e.g., Saccharomyces spp., Schizosaccharomyces spp., Pichia spp., Phaffia spp., Kluyveromyces spp., Candida spp., Talaromyces spp., Brettanomyces spp., Pachysolen spp., Debaryomyces spp., Yarrowia spp. and industrial polyploid yeast strains. Preferably the yeast strain is a S. cerevisiae strain. Other examples of fungi include Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp.

In some embodiments the cell is a bacterial cell and includes, without limitation, cells classified as Escherichia spp., Streptomyces spp., Zymonas spp., Acetobacter spp., Citrobacter spp., Synechocystis spp., Rhizobium spp., Clostridium spp., Corynebacterium spp., Streptococcus spp., Xanthomonas spp., Lactobacillus spp., Lactococcus spp., Bacillus spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Thermus spp., Stenotrophomonas spp., Chromobacterium spp., Sinorhizobium spp., Saccharopolyspora spp., Agrobacterium spp., and Pantoea spp. The bacterial cell can be a Gram-negative cell such as an Escherichia coli (E. coli) cell, or a Gram-positive cell such as a species of Bacillus. In certain embodiments, the cell is a BL21(DE3) cell. “Endogenous” bacterial cells refer to non-pathogenic bacteria that are part of a normal internal ecosystem such as bacterial flora. In some embodiments, bacterial cells of the present disclosure are anaerobic bacterial cells (e.g., cells that do not require oxygen for growth). Anaerobic bacterial cells include facultative anaerobic cells such as, for example, Escherichia coli, Shewanella oneidensis and Listeria monocytogenes. Anaerobic bacterial cells also include obligate anaerobic cells such as, for example, Bacteroides and Clostridium species. In humans, for example, anaerobic bacterial cells are most commonly found in the gastrointestinal tract.

In some embodiments, the cell is a mammalian cell. For example, in some embodiments, the genetic circuits are expressed in human cells, primate cells (e.g., VERO cells), rat cells (e.g., GH3 cells, OC23 cells) or mouse cells (e.g., MC3T3 cells). There are a variety of human cell lines, including, without limitation, human embryonic kidney (HEK) cells, HeLa cells, cancer cells from the National Cancer Institute's 60 cancer cell lines (NCI60), DU145 (prostate cancer) cells, LNCaP (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells, SHSYSY human neuroblastoma cells (cloned from a myeloma) and Saos-2 (bone cancer) cells. In some embodiments, engineered constructs are expressed in human embryonic kidney (HEK) cells (e.g., HEK 293 or HEK 293T cells).

In some embodiments, the cell is a stem cell (e.g., human stem cells) such as, for example, pluripotent stem cells (e.g., human pluripotent stem cells including human induced pluripotent stem cells (hiPSCs)). A “stem cell” refers to a cell with the ability to divide for indefinite periods in culture and to give rise to specialized cells. A “pluripotent stem cell” refers to a type of stem cell that is capable of differentiating into all tissues of an organism, but not alone capable of sustaining full organismal development. A “human induced pluripotent stem cell” refers to a somatic (e.g., mature or adult) cell that has been reprogrammed to an embryonic stem cell-like state by being forced to express genes and factors important for maintaining the defining properties of embryonic stem cells (see, e.g., Takahashi et al. (2006) Cell 126(4):663-76, incorporated herein by reference). Human induced pluripotent stem cell cells express stem cell markers and are capable of generating cells characteristic of all three germ layers (ectoderm, endoderm, mesoderm).

Also within the scope of the present disclosure are transgenic organisms comprising cells containing the genetic circuits described herein. Routine methods known in the art may be used to generate transgenic organisms comprising cells containing the genetic circuits described herein. In some embodiments, the cell is in a multicellular organism, for example a plant or a mammal. In some embodiments, the mammal is a rodent, such as a mouse or a rat.

Cells of the present disclosure, in some embodiments, are modified and may be referred to as recombinant cells. As used herein, a “recombinant cell” is a cell that contains an exogenous nucleic acid or a nucleic acid that does not occur in nature. In some embodiments, a cell contains an exogenous independently replicating nucleic acid (e.g., an engineered nucleic acid located on an episomal vector). In some embodiments, a cell is produced by introducing a foreign or exogenous nucleic acid (e.g., engineered nucleic acid) into a cell. Thus, provided herein are methods of introducing engineered nucleic acid into a cell. A nucleic acid may be introduced into a cell by conventional methods, such as, for example, electroporation (see, e.g., Heiser W. C. Transcription Factor Protocols: Methods in Molecular Biology™ (2000); 130: 117-134), chemical (e.g., calcium phosphate or lipid) transfection (see, e.g., Lewis W. H., et al., Somatic Cell Genet. (1980) 6(3):333-47; Chen C., et al., Mol Cell Biol. (1987) 7(8):2745-52), fusion with bacterial protoplasts containing recombinant plasmids (see, e.g., Schaffner W. Proc Natl Acad Sci USA. (1980) 77(4):2163-7), transduction (e.g., viral or phage transduction), conjugation, or microinjection (see, e.g., Capecchi M. R. Cell. (1980) 22(2 Pt 2):479-88). In some embodiments, the genetic constructs are introduced into a cell using plasmids or vectors. In some embodiments, the genetic constructs are introduced into a cell by a virus, such as lentiviruses or adenoviruses. Expressing the nucleic acid molecule may also be accomplished by integrating the nucleic acid molecule into the genome.

Engineered nucleic acids of the present disclosure may be transiently expressed or stably expressed. “Transient cell expression” refers to expression by a cell of a nucleic acid that is not integrated into the nuclear genome of the cell. By comparison, “stable cell expression” refers to expression by a cell of a nucleic acid that remains in the nuclear genome of the cell and its daughter cells. Typically, to achieve stable cell expression, a cell is co-transfected with a marker gene and an exogenous nucleic acid that is intended for stable expression in the cell. The marker gene gives the cell some selectable advantage (e.g., resistance to a toxin, antibiotic, or other factor). Few transfected cells will, by chance, have integrated the exogenous nucleic acid into their genome. If a toxin, for example, is then added to the cell culture, only those few cells with a toxin-resistant marker gene integrated into their genomes will be able to proliferate, while other cells will die. After applying this selective pressure for a period of time, only the cells with a stable transfection remain and can be cultured further. Expression of nucleic acids in transiently-transfected and/or stably-transfected cells may be constitutive or inducible. Inducible promoters for use as provided herein are described above.

Culture

Cells expressing the genetic constructs described herein may be cultured (e.g., maintained in cell culture) using conventional cell culture methods. For example, cells may be grown and maintained at an appropriate temperature and gas mixture (e.g., 37° C., 5% CO₂ for mammalian cells) in a cell incubator. In some embodiments, the cells may be incubated under specific conditions to induce a desired state of the cell, such as a development state, activation or disease state. Culture conditions may vary for each cell type. For example, cell growth media may vary in pH, glucose concentration, growth factors, and the presence of other nutrients. Growth factors used to supplement media are often derived from the serum of animal blood, such as fetal bovine serum (FBS), bovine calf serum, equine serum and/or porcine serum. In some embodiments, culture media used as provided herein may be commercially available and/or well-described (see, e.g., Birch J. R., R. G. Spier (Ed.) Encyclopedia of Cell Technology, Wiley. 411-424, 2000; Keen M. J. Cytotechnology (1995) 17:125-132; Zang, et al. Bio/Technology (1995) 13:389-392). In some embodiments, chemically defined media is used.

In some embodiments, fermentation processes for large-scale production of microbes expressing the genetic circuits described herein may be carried out in bioreactors. As used herein, the terms “bioreactor” and “fermentor”, which are interchangeably used, refer to an enclosure, or partial enclosure, in which a biological and/or chemical reaction takes place, at least part of which involves a living organism or part of a living organism. A “large-scale bioreactor” or “industrial-scale bioreactor” is a bioreactor that is used to generate a product on a commercial or quasi-commercial scale. Large scale bioreactors typically have volumes in the range of liters, hundreds of liters, thousands of liters, or more.

A bioreactor in accordance with aspects of the present disclosure may comprise a microbe or a microbe culture. In some embodiments, a bioreactor may comprise a spore and/or any kind of dormant cell type of any isolated microbe provided by aspects of the present disclosure, for example, in a dry state. In some embodiments, addition of a suitable carbohydrate source to such bioreactors may lead to activation of the dormant cell.

Some bioreactors according to aspects of this the present disclosure may include cell culture systems where microbes are in contact with moving liquids and/or gas bubbles. Microbes or microbe cultures in accordance with aspects of this the present disclosure may be grown in suspension or attached to solid phase carriers. Non-limiting examples of carrier systems include microcarriers (e.g., polymer spheres, microbeads, and microdisks that can be porous or non-porous), cross-linked beads (e.g., dextran) charged with specific chemical groups (e.g., tertiary amine groups), 2D microcarriers including cells trapped in nonporous polymer fibers, 3D carriers (e.g., carrier fibers, hollow fibers, multicartridge reactors, and semi-permeable membranes that can comprising porous fibers), microcarriers having reduced ion exchange capacity, encapsulation cells, capillaries, and aggregates. Carriers can be fabricated from materials such as dextran, gelatin, glass, and cellulose.

Industrial-scale processes in accordance with the present disclosure may be operated in continuous, semi-continuous or non-continuous modes. Non-limiting examples of operation modes in accordance with the present disclosure are batch, fed batch, extended batch, repetitive batch, draw/fill, rotating-wall, spinning flask, and/or perfusion mode of operation.

In some embodiments, bioreactors may be used that allow continuous or semi-continuous replenishment of the substrate stock, for example a carbohydrate source and/or continuous or semi-continuous separation of the product.

Non-limiting examples of bioreactors in accordance with the present disclosure are: stirred tank fermentors, bioreactors agitated by rotating mixing devices, chemostats, bioreactors agitated by shaking devices, airlift fermentors, packed-bed reactors, fixed-bed reactors, fluidized bed bioreactors, bioreactors employing wave induced agitation, centrifugal bioreactors, roller bottles, and hollow fiber bioreactors, roller apparatuses (for example benchtop, cart-mounted, and/or automated varieties), vertically-stacked plates, spinner flasks, stirring or rocking flasks, shaken multiwell plates, MD bottles, T-flasks, Roux bottles, multiple-surface tissue culture propagators, modified fermentors, and coated beads (e.g., beads coated with serum proteins, nitrocellulose, or carboxymethyl cellulose to prevent cell attachment).

Bioreactors and fermentors according to aspects of the present disclosure may, optionally, comprise a sensor and/or a control system to measure and/or adjust reaction parameters. Non-limiting examples of reaction parameters are: biological parameters, for example growth rate, cell size, cell number, cell density, cell type, or cell state, chemical parameters, for example pH, redox-potential, concentration of reaction substrate and/or product, concentration of dissolved gases, such as oxygen concentration and CO₂ concentration, nutrient concentrations, metabolite concentrations, glucose concentration, glutamine concentration, pyruvate concentration, apatite concentration, concentration of an oligopeptide, concentration of an amino acid, concentration of a vitamin, concentration of a hormone, concentration of an additive, serum concentration, ionic strength, concentration of an ion, relative humidity, molarity, osmolarity, concentration of other chemicals, for example buffering agents, adjuvants, or reaction by-products, physical/mechanical parameters, for example density, conductivity, degree of agitation, pressure, and flow rate, shear stress, shear rate, viscosity, color, turbidity, light absorption, mixing rate, conversion rate, as well as thermodynamic parameters, such as temperature, light intensity/quality etc.

Sensors able to measure parameters as described herein are well known to those of skill in the relevant mechanical and electronic arts. Control systems able to adjust the parameters in a bioreactor based on the inputs from a sensor as described herein are well known to those of skill in the art of bioreactor engineering.

The function and advantage of these and other embodiments will be more fully understood from the examples below. The following examples are intended to illustrate the benefits of the present invention, but do not exemplify the full scope of the invention.

Accordingly, it will be understood that the example section is not meant to limit the scope of the invention.

### EXAMPLES

### Example 1. Methods and Compositions for Designing RNA-Guided Genetic Circuits

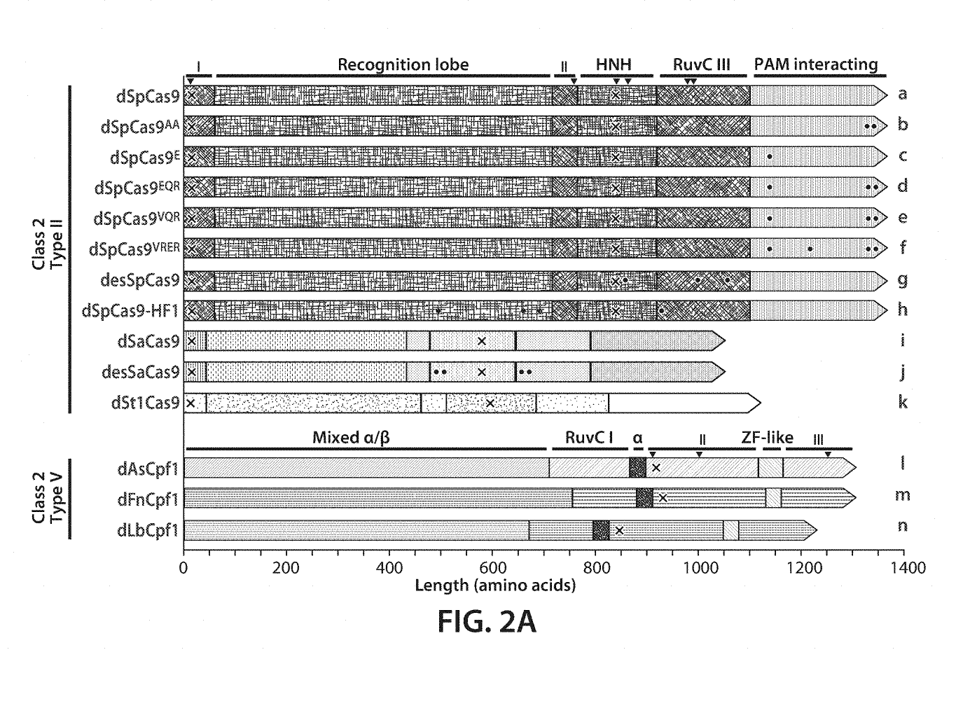

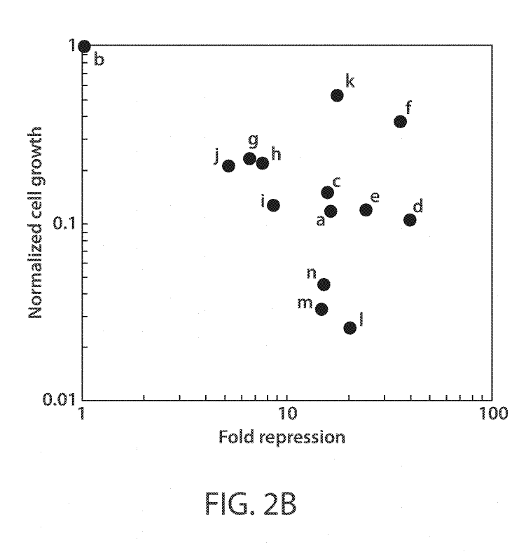

The technology described herein helps surmount the issues associated with RNA-guided genetic circuits. To overcome the poor cellular growth associated with catalytically-inactive RNA-guided nucleases, 14 mutants and orthologues of different RNA-guided endonucleases were screened for their ability to elicit high transcriptional repression but low cellular toxicity (FIGS. 2A-2B). Next, several strategies were tested to introduce non-linearity into the otherwise linear responses of the gates. Of these strategies, the use of “decoy” DNA sites to attract the guide RNA, and the use of sRNAs to degrade guide RNAs were successful (FIGS. 3A-3B).

To reduce the repeated DNA sequences that arise in each guide RNA, the guide RNAs were systematically mutated and tested for functionality. The result gave a map of every nucleotide that can be changed without reducing function, in order to increase the diversity of guide RNA sequences. This is broadly useful for any system which requires multiple guide RNAs to reduce recombination.

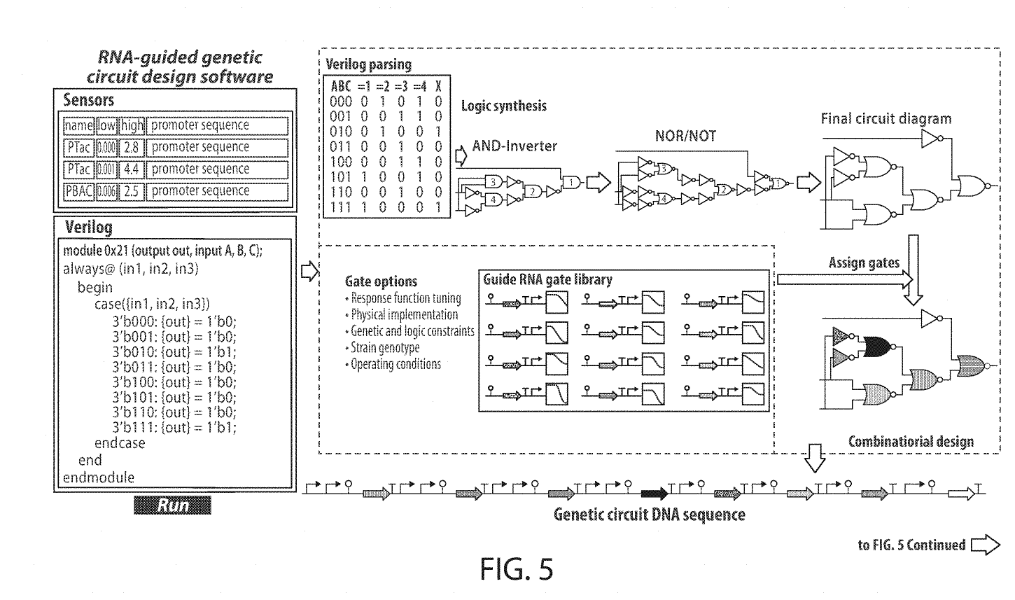

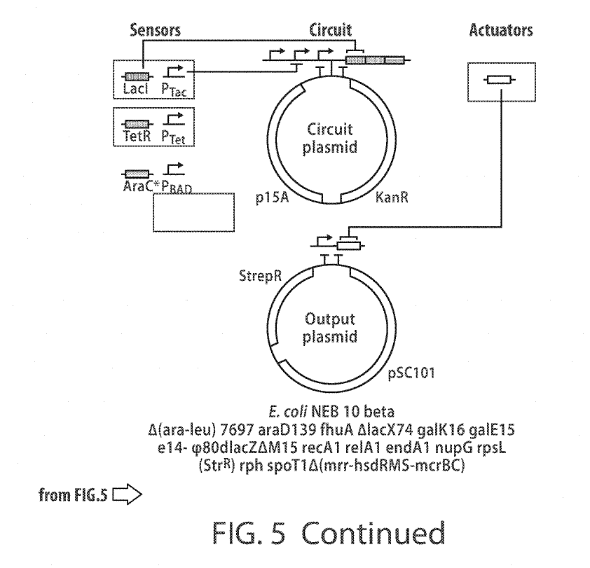

A solution for the host-dependent function of guide RNA genetic circuits was to design a strategy that used the heterologous phage T7 RNA polymerase instead of the host RNA polymerase for transcription of the guide RNAs (FIG. 4A). A software that automates the design of RNA-guided genetic circuits from a high-level programming language input by a user was developed (FIG. 5). The software takes user-defined code as input for the specification of the RNA-guided genetic circuit, and the input levels of the “sensors”. Using this information, the software first synthesizes an abstract wiring diagram for the circuit, and then assigns guide RNA gates to the circuit based on their response function tuning. Next, one or more 1D layouts of the nucleotide sequence for the circuit is generated using combinatorial design algorithms. The final DNA sequence is output along with predictions of the performance of the circuit.

Advantages and Improvements Over Existing Methods, Devices, or Materials

The current generation of RNA-guided genetic circuits are limited by several factors. First, the toxicity of the biomolecules can cause poor to negligible growth in some organisms and strains. Healthy cells are a prerequisite to many biotechnology applications, and the protein variants described here reduce toxicity. Second, the non-cooperative response of each gate causes the circuit's signal to deteriorate as the signal propagates through multiple gate layers until it is completely degraded. The response-function tuning techniques described here allow the signal to be maintained across gate layers. Third, the use of many guide RNAs in a circuit necessitates repeated use of certain DNA sequences, and this can drive homologous-recombination-mediated deletion of DNA. The guide RNA-diversification hotspots described here allow the similarity of guide RNAs to be minimized so that more of them can be used in a circuit.

The ability to rationally program cellular behaviors using the genetic circuits described herein has the potential to revolutionize several sectors of biotechnology. Precision microbiome therapeutics that can sense and respond to disease, bacterial strains optimized for bioreactor growth and chemical production, “smart” plants that can modulate gene expression in drought conditions, and materials which can grow and heal themselves are only a few examples of biotechnologies that would be enabled by synthetic genetic circuits, as the genetic circuits contemplated herein.

As engineered biological systems become more sophisticated, genetic circuits will play an increasingly important role and the technology described overcomes many of the challenges associated with the engineered biological systems currently available in the art. It is believed that this technology is analogous to the transistor, upon which the computer revolution was based.

Exemplary, representative sequences for catalytically inactive variants of dCas9 and dCpf1, PAM and guide RNA scaffolds contemplated herein include:

1. Variant Name: dSpCas9
Variant DNA:
Nucleotide Sequence
(SEQ ID NO: 1)
ATGGATAAGAAATACTCAATAGGCTTAGCTATCGGCACAAATAGCGTCGGAT
GGGCGGTGATCACTGATGAATATAAGGTTCCGTCTAAAAAGTTCAAGGTTCTGGGAA
ATACAGACCGCCACAGTATCAAAAAAAATCTTATAGGGGCTCTTTTATTTGACAGTG
GAGAGACAGCGGAAGCGACTCGTCTCAAACGGACAGCTCGTAGAAGGTATACACGTC
GGAAGAATCGTATTTGTTATCTACAGGAGATTTTTTCAAATGAGATGGCGAAAGTAG
ATGATAGTTTCTTTCATCGACTTGAAGAGTCTTTTTTGGTGGAAGAAGACAAGAAGC
ATGAACGTCATCCTATTTTTGGAAATATAGTAGATGAAGTTGCTTATCATGAGAAAT
ATCCAACTATCTATCATCTGCGAAAAAAATTGGTAGATTCTACTGATAAAGCGGATT
TGCGCTTAATCTATTTGGCCTTAGCGCATATGATTAAGTTTCGTGGTCATTTTTTGA
TTGAGGGAGATTTAAATCCTGATAATAGTGATGTGGACAAACTATTTATCCAGTTGG
TACAAACCTACAATCAATTATTTGAAGAAAACCCTATTAACGCAAGTGGAGTAGATG
CTAAAGCGATTCTTTCTGCACGATTGAGTAAATCAAGACGATTAGAAAATCTCATTG
CTCAGCTCCCCGGTGAGAAGAAAAATGGCTTATTTGGGAATCTCATTGCTTTGTCAT
TGGGTTTGACCCCTAATTTTAAATCAAATTTTGATTTGGCAGAAGATGCTAAATTAC
AGCTTTCAAAAGATACTTACGATGATGATTTAGATAATTTATTGGCGCAAATTGGAG
ATCAATATGCTGATTTGTTTTTGGCAGCTAAGAATTTATCAGATGCTATTTTACTTT
CAGATATCCTAAGAGTAAATACTGAAATAACTAAGGCTCCCCTATCAGCTTCAATGA
TTAAACGCTACGATGAACATCATCAAGACTTGACTCTTTTAAAAGCTTTAGTTCGAC
AACAACTTCCAGAAAAGTATAAAGAAATCTTTTTTGATCAATCAAAAAACGGATATG
CAGGTTATATTGATGGGGGAGCTAGCCAAGAAGAATTTTATAAATTTATCAAACCAA
TTTTAGAAAAAATGGATGGTACTGAGGAATTATTGGTGAAACTAAATCGTGAAGATT
TGCTGCGCAAGCAACGGACCTTTGACAACGGCTCTATTCCCCATCAAATTCACTTGG
GTGAGCTGCATGCTATTTTGAGAAGACAAGAAGACTTTTATCCATTTTTAAAAGACA
ATCGTGAGAAGATTGAAAAAATCTTGACTTTTCGAATTCCTTATTATGTTGGTCCAT
TGGCGCGTGGCAATAGTCGTTTTGCATGGATGACTCGGAAGTCTGAAGAAACAATTA
CCCCATGGAATTTTGAAGAAGTTGTCGATAAAGGTGCTTCAGCTCAATCATTTATTG
AACGCATGACAAACTTTGATAAAAATCTTCCAAATGAAAAAGTACTACCAAAACATA
GTTTGCTTTATGAGTATTTTACGGTTTATAACGAATTGACAAAGGTCAAATATGTTA
CTGAAGGAATGCGAAAACCAGCATTTCTTTCAGGTGAACAGAAGAAAGCCATTGTTG
ATTTACTCTTCAAAACAAATCGAAAAGTAACCGTTAAGCAATTAAAAGAAGATTATT
TCAAAAAAATAGAATGTTTTGATAGTGTTGAAATTTCAGGAGTTGAAGATAGATTTA
ATGCTTCATTAGGTACCTACCATGATTTGCTAAAAATTATTAAAGATAAAGATTTTT
TGGATAATGAAGAAAATGAAGATATCTTAGAGGATATTGTTTTAACATTGACCTTAT
TTGAAGATAGGGAGATGATTGAGGAAAGACTTAAAACATATGCTCACCTCTTTGATG
ATAAGGTGATGAAACAGCTTAAACGTCGCCGTTATACTGGTTGGGGACGTTTGTCTC
GAAAATTGATTAATGGTATTAGGGATAAGCAATCTGGCAAAACAATATTAGATTTTT
TGAAATCAGATGGTTTTGCCAATCGCAATTTTATGCAGCTGATCCATGATGATAGTT
TGACATTTAAAGAAGACATTCAAAAAGCACAAGTGTCTGGACAAGGCGATAGTTTAC
ATGAACATATTGCAAATTTAGCTGGTAGCCCTGCTATTAAAAAAGGTATTTTACAGA
CTGTAAAAGTTGTTGATGAATTGGTCAAAGTAATGGGGCGGCATAAGCCAGAAAATA
TCGTTATTGAAATGGCACGTGAAAATCAGACAACTCAAAAGGGCCAGAAAAATTCGC
GAGAGCGTATGAAACGAATCGAAGAAGGTATCAAAGAATTAGGAAGTCAGATTCTTA
AAGAGCATCCTGTTGAAAATACTCAATTGCAAAATGAAAAGCTCTATCTCTATTATC
TCCAAAATGGAAGAGACATGTATGTGGACCAAGAATTAGATATTAATCGTTTAAGTG
ATTATGATGTCGATGCCATTGTTCCACAAAGTTTCCTTAAAGACGATTCAATAGACA
ATAAGGTCTTAACGCGTTCTGATAAAAATCGTGGTAAATCGGATAACGTTCCAAGTG
AAGAAGTAGTCAAAAAGATGAAAAACTATTGGAGACAACTTCTAAACGCCAAGTTAA
TCACTCAACGTAAGTTTGATAATTTAACGAAAGCTGAACGTGGAGGTTTGAGTGAAC
TTGATAAAGCTGGTTTTATCAAACGCCAATTGGTTGAAACTCGCCAAATCACTAAGC
ATGTGGCACAAATTTTGGATAGTCGCATGAATACTAAATACGATGAAAATGATAAAC
TTATTCGAGAGGTTAAAGTGATTACCTTAAAATCTAAATTAGTTTCTGACTTCCGAA
AAGATTTCCAATTCTATAAAGTACGTGAGATTAACAATTACCATCATGCCCATGATG
CGTATCTAAATGCCGTCGTTGGAACTGCTTTGATTAAGAAATATCCAAAACTTGAAT
CGGAGTTTGTCTATGGTGATTATAAAGTTTATGATGTTCGTAAAATGATTGCTAAGT
CTGAGCAAGAAATAGGCAAAGCAACCGCAAAATATTTCTTTTACTCTAATATCATGA
ACTTCTTCAAAACAGAAATTACACTTGCAAATGGAGAGATTCGCAAACGCCCTCTAA
TCGAAACTAATGGGGAAACTGGAGAAATTGTCTGGGATAAAGGGCGAGATTTTGCCA
CAGTGCGCAAAGTATTGTCCATGCCCCAAGTCAATATTGTCAAGAAAACAGAAGTAC
AGACAGGCGGATTCTCCAAGGAGTCAATTTTACCAAAAAGAAATTCGGACAAGCTTA
TTGCTCGTAAAAAAGACTGGGATCCAAAAAAATATGGTGGTTTTGATAGTCCAACGG
TAGCTTATTCAGTCCTAGTGGTTGCTAAGGTGGAAAAAGGGAAATCGAAGAAGTTAA
AATCCGTTAAAGAGTTACTAGGGATCACAATTATGGAAAGAAGTTCCTTTGAAAAAA
ATCCGATTGACTTTTTAGAAGCTAAAGGATATAAGGAAGTTAAAAAAGACTTAATCA
TTAAACTACCTAAATATAGTCTTTTTGAGTTAGAAAACGGTCGTAAACGGATGCTGG
CTAGTGCCGGAGAATTACAAAAAGGAAATGAGCTGGCTCTGCCAAGCAAATATGTGA
ATTTTTTATATTTAGCTAGTCATTATGAAAAGTTGAAGGGTAGTCCAGAAGATAACG
AACAAAAACAATTGTTTGTGGAGCAGCATAAGCATTATTTAGATGAGATTATTGAGC
AAATCAGTGAATTTTCTAAGCGTGTTATTTTAGCAGATGCCAATTTAGATAAAGTTC
TTAGTGCATATAACAAACATAGAGACAAACCAATACGTGAACAAGCAGAAAATATTA
TTCATTTATTTACGTTGACGAATCTTGGAGCTCCCGCTGCTTTTAAATATTTTGATA
CAACAATTGATCGTAAACGATATACGTCTACAAAAGAAGTTTTAGATGCCACTCTTA
TCCATCAATCCATCACTGGTCTTTATGAAACACGCATTGATTTGAGTCAGCTAGGAG
GTGAC
PAM Position: 3′
PAM: NRG
Guide RNA Scaffold:
Nucleotide Sequence
(SEQ ID NO: 2)
GTTTTAGAGCTAGAAATAGCAAGTTAAAATAAGGCTAGTCCGTTATCAACTT
GAAAAAGTGGCACCGAGTCGGTGCTTTTTTT
Reference: Qi, et al. (2013) Cell 152.5:1173-83.
2. Variant Name: dSpCas9(E)
Variant DNA:
Nucleotide Sequence
(SEQ ID NO: 3)
ATGGATAAGAAATACTCAATAGGCTTAGCTATCGGCACAAATAGCGTCGGAT
GGGCGGTGATCACTGATGAATATAAGGTTCCGTCTAAAAAGTTCAAGGTTCTGGGAA
ATACAGACCGCCACAGTATCAAAAAAAATCTTATAGGGGCTCTTTTATTTGACAGTG
GAGAGACAGCGGAAGCGACTCGTCTCAAACGGACAGCTCGTAGAAGGTATACACGTC
GGAAGAATCGTATTTGTTATCTACAGGAGATTTTTTCAAATGAGATGGCGAAAGTAG
ATGATAGTTTCTTTCATCGACTTGAAGAGTCTTTTTTGGTGGAAGAAGACAAGAAGC
ATGAACGTCATCCTATTTTTGGAAATATAGTAGATGAAGTTGCTTATCATGAGAAAT
ATCCAACTATCTATCATCTGCGAAAAAAATTGGTAGATTCTACTGATAAAGCGGATT
TGCGCTTAATCTATTTGGCCTTAGCGCATATGATTAAGTTTCGTGGTCATTTTTTGA
TTGAGGGAGATTTAAATCCTGATAATAGTGATGTGGACAAACTATTTATCCAGTTGG
TACAAACCTACAATCAATTATTTGAAGAAAACCCTATTAACGCAAGTGGAGTAGATG
CTAAAGCGATTCTTTCTGCACGATTGAGTAAATCAAGACGATTAGAAAATCTCATTG
CTCAGCTCCCCGGTGAGAAGAAAAATGGCTTATTTGGGAATCTCATTGCTTTGTCAT
TGGGTTTGACCCCTAATTTTAAATCAAATTTTGATTTGGCAGAAGATGCTAAATTAC
AGCTTTCAAAAGATACTTACGATGATGATTTAGATAATTTATTGGCGCAAATTGGAG
ATCAATATGCTGATTTGTTTTTGGCAGCTAAGAATTTATCAGATGCTATTTTACTTT
CAGATATCCTAAGAGTAAATACTGAAATAACTAAGGCTCCCCTATCAGCTTCAATGA
TTAAACGCTACGATGAACATCATCAAGACTTGACTCTTTTAAAAGCTTTAGTTCGAC
AACAACTTCCAGAAAAGTATAAAGAAATCTTTTTTGATCAATCAAAAAACGGATATG
CAGGTTATATTGATGGGGGAGCTAGCCAAGAAGAATTTTATAAATTTATCAAACCAA
TTTTAGAAAAAATGGATGGTACTGAGGAATTATTGGTGAAACTAAATCGTGAAGATT
TGCTGCGCAAGCAACGGACCTTTGACAACGGCTCTATTCCCCATCAAATTCACTTGG
GTGAGCTGCATGCTATTTTGAGAAGACAAGAAGACTTTTATCCATTTTTAAAAGACA
ATCGTGAGAAGATTGAAAAAATCTTGACTTTTCGAATTCCTTATTATGTTGGTCCAT
TGGCGCGTGGCAATAGTCGTTTTGCATGGATGACTCGGAAGTCTGAAGAAACAATTA
CCCCATGGAATTTTGAAGAAGTTGTCGATAAAGGTGCTTCAGCTCAATCATTTATTG
AACGCATGACAAACTTTGATAAAAATCTTCCAAATGAAAAAGTACTACCAAAACATA
GTTTGCTTTATGAGTATTTTACGGTTTATAACGAATTGACAAAGGTCAAATATGTTA
CTGAAGGAATGCGAAAACCAGCATTTCTTTCAGGTGAACAGAAGAAAGCCATTGTTG
ATTTACTCTTCAAAACAAATCGAAAAGTAACCGTTAAGCAATTAAAAGAAGATTATT
TCAAAAAAATAGAATGTTTTGATAGTGTTGAAATTTCAGGAGTTGAAGATAGATTTA
ATGCTTCATTAGGTACCTACCATGATTTGCTAAAAATTATTAAAGATAAAGATTTTT
TGGATAATGAAGAAAATGAAGATATCTTAGAGGATATTGTTTTAACATTGACCTTAT
TTGAAGATAGGGAGATGATTGAGGAAAGACTTAAAACATATGCTCACCTCTTTGATG
ATAAGGTGATGAAACAGCTTAAACGTCGCCGTTATACTGGTTGGGGACGTTTGTCTC
GAAAATTGATTAATGGTATTAGGGATAAGCAATCTGGCAAAACAATATTAGATTTTT
TGAAATCAGATGGTTTTGCCAATCGCAATTTTATGCAGCTGATCCATGATGATAGTT
TGACATTTAAAGAAGACATTCAAAAAGCACAAGTGTCTGGACAAGGCGATAGTTTAC
ATGAACATATTGCAAATTTAGCTGGTAGCCCTGCTATTAAAAAAGGTATTTTACAGA
CTGTAAAAGTTGTTGATGAATTGGTCAAAGTAATGGGGCGGCATAAGCCAGAAAATA
TCGTTATTGAAATGGCACGTGAAAATCAGACAACTCAAAAGGGCCAGAAAAATTCGC
GAGAGCGTATGAAACGAATCGAAGAAGGTATCAAAGAATTAGGAAGTCAGATTCTTA
AAGAGCATCCTGTTGAAAATACTCAATTGCAAAATGAAAAGCTCTATCTCTATTATC
TCCAAAATGGAAGAGACATGTATGTGGACCAAGAATTAGATATTAATCGTTTAAGTG
ATTATGATGTCGATGCCATTGTTCCACAAAGTTTCCTTAAAGACGATTCAATAGACA
ATAAGGTCTTAACGCGTTCTGATAAAAATCGTGGTAAATCGGATAACGTTCCAAGTG
AAGAAGTAGTCAAAAAGATGAAAAACTATTGGAGACAACTTCTAAACGCCAAGTTAA
TCACTCAACGTAAGTTTGATAATTTAACGAAAGCTGAACGTGGAGGTTTGAGTGAAC
TTGATAAAGCTGGTTTTATCAAACGCCAATTGGTTGAAACTCGCCAAATCACTAAGC
ATGTGGCACAAATTTTGGATAGTCGCATGAATACTAAATACGATGAAAATGATAAAC
TTATTCGAGAGGTTAAAGTGATTACCTTAAAATCTAAATTAGTTTCTGACTTCCGAA
AAGATTTCCAATTCTATAAAGTACGTGAGATTAACAATTACCATCATGCCCATGATG
CGTATCTAAATGCCGTCGTTGGAACTGCTTTGATTAAGAAATATCCAAAACTTGAAT
CGGAGTTTGTCTATGGTGATTATAAAGTTTATGATGTTCGTAAAATGATTGCTAAGT
CTGAGCAAGAAATAGGCAAAGCAACCGCAAAATATTTCTTTTACTCTAATATCATGA
ACTTCTTCAAAACAGAAATTACACTTGCAAATGGAGAGATTCGCAAACGCCCTCTAA
TCGAAACTAATGGGGAAACTGGAGAAATTGTCTGGGATAAAGGGCGAGATTTTGCCA
CAGTGCGCAAAGTATTGTCCATGCCCCAAGTCAATATTGTCAAGAAAACAGAAGTAC
AGACAGGCGGATTCTCCAAGGAGTCAATTTTACCAAAAAGAAATTCGGACAAGCTTA
TTGCTCGTAAAAAAGACTGGGATCCAAAAAAATATGGTGGTTTTgaaAGTCCAACGG
TAGCTTATTCAGTCCTAGTGGTTGCTAAGGTGGAAAAAGGGAAATCGAAGAAGTTAA
AATCCGTTAAAGAGTTACTAGGGATCACAATTATGGAAAGAAGTTCCTTTGAAAAAA
ATCCGATTGACTTTTTAGAAGCTAAAGGATATAAGGAAGTTAAAAAAGACTTAATCA
TTAAACTACCTAAATATAGTCTTTTTGAGTTAGAAAACGGTCGTAAACGGATGCTGG
CTAGTGCCGGAGAATTACAAAAAGGAAATGAGCTGGCTCTGCCAAGCAAATATGTGA
ATTTTTTATATTTAGCTAGTCATTATGAAAAGTTGAAGGGTAGTCCAGAAGATAACG
AACAAAAACAATTGTTTGTGGAGCAGCATAAGCATTATTTAGATGAGATTATTGAGC
AAATCAGTGAATTTTCTAAGCGTGTTATTTTAGCAGATGCCAATTTAGATAAAGTTC
TTAGTGCATATAACAAACATAGAGACAAACCAATACGTGAACAAGCAGAAAATATTA
TTCATTTATTTACGTTGACGAATCTTGGAGCTCCCGCTGCTTTTAAATATTTTGATA
CAACAATTGATCGTAAACGATATACGTCTACAAAAGAAGTTTTAGATGCCACTCTTA
TCCATCAATCCATCACTGGTCTTTATGAAACACGCATTGATTTGAGTCAGCTAGGAG
GTGAC
PAM Position: 3′
PAM: NGG
Guide RNA Scaffold:
Nucleotide Sequence
(SEQ ID NO: 4)
GTTTTAGAGCTAGAAATAGCAAGTTAAAATAAGGCTAGTCCGTTATCAACTT
GAAAAAGTGGCACCGAGTCGGTGCTTTTTTT
Reference: Kleinstiver, et al. (2015) Nature 523.7561:481-5.
3. Variant Name: dSpCas9(VRER)
Variant DNA:
Nucleotide Sequence
(SEQ ID NO: 5)
ATGGATAAGAAATACTCAATAGGCTTAGCTATCGGCACAAATAGCGTCGGAT
GGGCGGTGATCACTGATGAATATAAGGTTCCGTCTAAAAAGTTCAAGGTTCTGGGAA
ATACAGACCGCCACAGTATCAAAAAAAATCTTATAGGGGCTCTTTTATTTGACAGTG
GAGAGACAGCGGAAGCGACTCGTCTCAAACGGACAGCTCGTAGAAGGTATACACGTC
GGAAGAATCGTATTTGTTATCTACAGGAGATTTTTTCAAATGAGATGGCGAAAGTAG
ATGATAGTTTCTTTCATCGACTTGAAGAGTCTTTTTTGGTGGAAGAAGACAAGAAGC
ATGAACGTCATCCTATTTTTGGAAATATAGTAGATGAAGTTGCTTATCATGAGAAAT
ATCCAACTATCTATCATCTGCGAAAAAAATTGGTAGATTCTACTGATAAAGCGGATT
TGCGCTTAATCTATTTGGCCTTAGCGCATATGATTAAGTTTCGTGGTCATTTTTTGA
TTGAGGGAGATTTAAATCCTGATAATAGTGATGTGGACAAACTATTTATCCAGTTGG
TACAAACCTACAATCAATTATTTGAAGAAAACCCTATTAACGCAAGTGGAGTAGATG
CTAAAGCGATTCTTTCTGCACGATTGAGTAAATCAAGACGATTAGAAAATCTCATTG
CTCAGCTCCCCGGTGAGAAGAAAAATGGCTTATTTGGGAATCTCATTGCTTTGTCAT
TGGGTTTGACCCCTAATTTTAAATCAAATTTTGATTTGGCAGAAGATGCTAAATTAC
AGCTTTCAAAAGATACTTACGATGATGATTTAGATAATTTATTGGCGCAAATTGGAG
ATCAATATGCTGATTTGTTTTTGGCAGCTAAGAATTTATCAGATGCTATTTTACTTT
CAGATATCCTAAGAGTAAATACTGAAATAACTAAGGCTCCCCTATCAGCTTCAATGA
TTAAACGCTACGATGAACATCATCAAGACTTGACTCTTTTAAAAGCTTTAGTTCGAC
AACAACTTCCAGAAAAGTATAAAGAAATCTTTTTTGATCAATCAAAAAACGGATATG
CAGGTTATATTGATGGGGGAGCTAGCCAAGAAGAATTTTATAAATTTATCAAACCAA
TTTTAGAAAAAATGGATGGTACTGAGGAATTATTGGTGAAACTAAATCGTGAAGATT
TGCTGCGCAAGCAACGGACCTTTGACAACGGCTCTATTCCCCATCAAATTCACTTGG
GTGAGCTGCATGCTATTTTGAGAAGACAAGAAGACTTTTATCCATTTTTAAAAGACA
ATCGTGAGAAGATTGAAAAAATCTTGACTTTTCGAATTCCTTATTATGTTGGTCCAT
TGGCGCGTGGCAATAGTCGTTTTGCATGGATGACTCGGAAGTCTGAAGAAACAATTA
CCCCATGGAATTTTGAAGAAGTTGTCGATAAAGGTGCTTCAGCTCAATCATTTATTG
AACGCATGACAAACTTTGATAAAAATCTTCCAAATGAAAAAGTACTACCAAAACATA
GTTTGCTTTATGAGTATTTTACGGTTTATAACGAATTGACAAAGGTCAAATATGTTA
CTGAAGGAATGCGAAAACCAGCATTTCTTTCAGGTGAACAGAAGAAAGCCATTGTTG
ATTTACTCTTCAAAACAAATCGAAAAGTAACCGTTAAGCAATTAAAAGAAGATTATT
TCAAAAAAATAGAATGTTTTGATAGTGTTGAAATTTCAGGAGTTGAAGATAGATTTA
ATGCTTCATTAGGTACCTACCATGATTTGCTAAAAATTATTAAAGATAAAGATTTTT
TGGATAATGAAGAAAATGAAGATATCTTAGAGGATATTGTTTTAACATTGACCTTAT
TTGAAGATAGGGAGATGATTGAGGAAAGACTTAAAACATATGCTCACCTCTTTGATG
ATAAGGTGATGAAACAGCTTAAACGTCGCCGTTATACTGGTTGGGGACGTTTGTCTC
GAAAATTGATTAATGGTATTAGGGATAAGCAATCTGGCAAAACAATATTAGATTTTT
TGAAATCAGATGGTTTTGCCAATCGCAATTTTATGCAGCTGATCCATGATGATAGTT
TGACATTTAAAGAAGACATTCAAAAAGCACAAGTGTCTGGACAAGGCGATAGTTTAC
ATGAACATATTGCAAATTTAGCTGGTAGCCCTGCTATTAAAAAAGGTATTTTACAGA
CTGTAAAAGTTGTTGATGAATTGGTCAAAGTAATGGGGCGGCATAAGCCAGAAAATA
TCGTTATTGAAATGGCACGTGAAAATCAGACAACTCAAAAGGGCCAGAAAAATTCGC
GAGAGCGTATGAAACGAATCGAAGAAGGTATCAAAGAATTAGGAAGTCAGATTCTTA
AAGAGCATCCTGTTGAAAATACTCAATTGCAAAATGAAAAGCTCTATCTCTATTATC
TCCAAAATGGAAGAGACATGTATGTGGACCAAGAATTAGATATTAATCGTTTAAGTG
ATTATGATGTCGATGCCATTGTTCCACAAAGTTTCCTTAAAGACGATTCAATAGACA
ATAAGGTCTTAACGCGTTCTGATAAAAATCGTGGTAAATCGGATAACGTTCCAAGTG
AAGAAGTAGTCAAAAAGATGAAAAACTATTGGAGACAACTTCTAAACGCCAAGTTAA
TCACTCAACGTAAGTTTGATAATTTAACGAAAGCTGAACGTGGAGGTTTGAGTGAAC
TTGATAAAGCTGGTTTTATCAAACGCCAATTGGTTGAAACTCGCCAAATCACTAAGC
ATGTGGCACAAATTTTGGATAGTCGCATGAATACTAAATACGATGAAAATGATAAAC
TTATTCGAGAGGTTAAAGTGATTACCTTAAAATCTAAATTAGTTTCTGACTTCCGAA
AAGATTTCCAATTCTATAAAGTACGTGAGATTAACAATTACCATCATGCCCATGATG
CGTATCTAAATGCCGTCGTTGGAACTGCTTTGATTAAGAAATATCCAAAACTTGAAT
CGGAGTTTGTCTATGGTGATTATAAAGTTTATGATGTTCGTAAAATGATTGCTAAGT
CTGAGCAAGAAATAGGCAAAGCAACCGCAAAATATTTCTTTTACTCTAATATCATGA
ACTTCTTCAAAACAGAAATTACACTTGCAAATGGAGAGATTCGCAAACGCCCTCTAA
TCGAAACTAATGGGGAAACTGGAGAAATTGTCTGGGATAAAGGGCGAGATTTTGCCA
CAGTGCGCAAAGTATTGTCCATGCCCCAAGTCAATATTGTCAAGAAAACAGAAGTAC
AGACAGGCGGATTCTCCAAGGAGTCAATTTTACCAAAAAGAAATTCGGACAAGCTTA
TTGCTCGTAAAAAAGACTGGGATCCAAAAAAATATGGTGGTTTTGTGAGTCCAACGG
TAGCTTATTCAGTCCTAGTGGTTGCTAAGGTGGAAAAAGGGAAATCGAAGAAGTTAA
AATCCGTTAAAGAGTTACTAGGGATCACAATTATGGAAAGAAGTTCCTTTGAAAAAA
ATCCGATTGACTTTTTAGAAGCTAAAGGATATAAGGAAGTTAAAAAAGACTTAATCA
TTAAACTACCTAAATATAGTCTTTTTGAGTTAGAAAACGGTCGTAAACGGATGCTGG
CTAGTGCCCGTGAATTACAAAAAGGAAATGAGCTGGCTCTGCCAAGCAAATATGTGA
ATTTTTTATATTTAGCTAGTCATTATGAAAAGTTGAAGGGTAGTCCAGAAGATAACG
AACAAAAACAATTGTTTGTGGAGCAGCATAAGCATTATTTAGATGAGATTATTGAGC
AAATCAGTGAATTTTCTAAGCGTGTTATTTTAGCAGATGCCAATTTAGATAAAGTTC
TTAGTGCATATAACAAACATAGAGACAAACCAATACGTGAACAAGCAGAAAATATTA
TTCATTTATTTACGTTGACGAATCTTGGAGCTCCCGCTGCTTTTAAATATTTTGATA
CAACAATTGATCGTAAAGAATATCGTTCTACAAAAGAAGTTTTAGATGCCACTCTTA
TCCATCAATCCATCACTGGTCTTTATGAAACACGCATTGATTTGAGTCAGCTAGGAG
GTGAC
PAM Position: 3′
PAM: NGCG
Guide RNA Scaffold:
Nucleotide Sequence
(SEQ ID NO: 6)
GTTTTAGAGCTAGAAATAGCAAGTTAAAATAAGGCTAGTCCGTTATCAACTT
GAAAAAGTGGCACCGAGTCGGTGCTTTTTTT
Reference: Kleinstiver, et al. (2015) Nature 523.7561:481-5.
4. Variant Name: dSpCas9(VQR)
Variant DNA:
Nucleotide Sequence
(SEQ ID NO: 7)
ATGGATAAGAAATACTCAATAGGCTTAGCTATCGGCACAAATAGCGTCGGAT
GGGCGGTGATCACTGATGAATATAAGGTTCCGTCTAAAAAGTTCAAGGTTCTGGGAA
ATACAGACCGCCACAGTATCAAAAAAAATCTTATAGGGGCTCTTTTATTTGACAGTG
GAGAGACAGCGGAAGCGACTCGTCTCAAACGGACAGCTCGTAGAAGGTATACACGTC
GGAAGAATCGTATTTGTTATCTACAGGAGATTTTTTCAAATGAGATGGCGAAAGTAG
ATGATAGTTTCTTTCATCGACTTGAAGAGTCTTTTTTGGTGGAAGAAGACAAGAAGC
ATGAACGTCATCCTATTTTTGGAAATATAGTAGATGAAGTTGCTTATCATGAGAAAT
ATCCAACTATCTATCATCTGCGAAAAAAATTGGTAGATTCTACTGATAAAGCGGATT
TGCGCTTAATCTATTTGGCCTTAGCGCATATGATTAAGTTTCGTGGTCATTTTTTGA
TTGAGGGAGATTTAAATCCTGATAATAGTGATGTGGACAAACTATTTATCCAGTTGG
TACAAACCTACAATCAATTATTTGAAGAAAACCCTATTAACGCAAGTGGAGTAGATG
CTAAAGCGATTCTTTCTGCACGATTGAGTAAATCAAGACGATTAGAAAATCTCATTG
CTCAGCTCCCCGGTGAGAAGAAAAATGGCTTATTTGGGAATCTCATTGCTTTGTCAT
TGGGTTTGACCCCTAATTTTAAATCAAATTTTGATTTGGCAGAAGATGCTAAATTAC
AGCTTTCAAAAGATACTTACGATGATGATTTAGATAATTTATTGGCGCAAATTGGAG
ATCAATATGCTGATTTGTTTTTGGCAGCTAAGAATTTATCAGATGCTATTTTACTTT
CAGATATCCTAAGAGTAAATACTGAAATAACTAAGGCTCCCCTATCAGCTTCAATGA
TTAAACGCTACGATGAACATCATCAAGACTTGACTCTTTTAAAAGCTTTAGTTCGAC
AACAACTTCCAGAAAAGTATAAAGAAATCTTTTTTGATCAATCAAAAAACGGATATG
CAGGTTATATTGATGGGGGAGCTAGCCAAGAAGAATTTTATAAATTTATCAAACCAA
TTTTAGAAAAAATGGATGGTACTGAGGAATTATTGGTGAAACTAAATCGTGAAGATT
TGCTGCGCAAGCAACGGACCTTTGACAACGGCTCTATTCCCCATCAAATTCACTTGG
GTGAGCTGCATGCTATTTTGAGAAGACAAGAAGACTTTTATCCATTTTTAAAAGACA
ATCGTGAGAAGATTGAAAAAATCTTGACTTTTCGAATTCCTTATTATGTTGGTCCAT
TGGCGCGTGGCAATAGTCGTTTTGCATGGATGACTCGGAAGTCTGAAGAAACAATTA
CCCCATGGAATTTTGAAGAAGTTGTCGATAAAGGTGCTTCAGCTCAATCATTTATTG
AACGCATGACAAACTTTGATAAAAATCTTCCAAATGAAAAAGTACTACCAAAACATA
GTTTGCTTTATGAGTATTTTACGGTTTATAACGAATTGACAAAGGTCAAATATGTTA
CTGAAGGAATGCGAAAACCAGCATTTCTTTCAGGTGAACAGAAGAAAGCCATTGTTG
ATTTACTCTTCAAAACAAATCGAAAAGTAACCGTTAAGCAATTAAAAGAAGATTATT
TCAAAAAAATAGAATGTTTTGATAGTGTTGAAATTTCAGGAGTTGAAGATAGATTTA
ATGCTTCATTAGGTACCTACCATGATTTGCTAAAAATTATTAAAGATAAAGATTTTT
TGGATAATGAAGAAAATGAAGATATCTTAGAGGATATTGTTTTAACATTGACCTTAT
TTGAAGATAGGGAGATGATTGAGGAAAGACTTAAAACATATGCTCACCTCTTTGATG
ATAAGGTGATGAAACAGCTTAAACGTCGCCGTTATACTGGTTGGGGACGTTTGTCTC
GAAAATTGATTAATGGTATTAGGGATAAGCAATCTGGCAAAACAATATTAGATTTTT
TGAAATCAGATGGTTTTGCCAATCGCAATTTTATGCAGCTGATCCATGATGATAGTT
TGACATTTAAAGAAGACATTCAAAAAGCACAAGTGTCTGGACAAGGCGATAGTTTAC
ATGAACATATTGCAAATTTAGCTGGTAGCCCTGCTATTAAAAAAGGTATTTTACAGA
CTGTAAAAGTTGTTGATGAATTGGTCAAAGTAATGGGGCGGCATAAGCCAGAAAATA
TCGTTATTGAAATGGCACGTGAAAATCAGACAACTCAAAAGGGCCAGAAAAATTCGC
GAGAGCGTATGAAACGAATCGAAGAAGGTATCAAAGAATTAGGAAGTCAGATTCTTA
AAGAGCATCCTGTTGAAAATACTCAATTGCAAAATGAAAAGCTCTATCTCTATTATC
TCCAAAATGGAAGAGACATGTATGTGGACCAAGAATTAGATATTAATCGTTTAAGTG
ATTATGATGTCGATGCCATTGTTCCACAAAGTTTCCTTAAAGACGATTCAATAGACA
ATAAGGTCTTAACGCGTTCTGATAAAAATCGTGGTAAATCGGATAACGTTCCAAGTG
AAGAAGTAGTCAAAAAGATGAAAAACTATTGGAGACAACTTCTAAACGCCAAGTTAA
TCACTCAACGTAAGTTTGATAATTTAACGAAAGCTGAACGTGGAGGTTTGAGTGAAC
TTGATAAAGCTGGTTTTATCAAACGCCAATTGGTTGAAACTCGCCAAATCACTAAGC
ATGTGGCACAAATTTTGGATAGTCGCATGAATACTAAATACGATGAAAATGATAAAC
TTATTCGAGAGGTTAAAGTGATTACCTTAAAATCTAAATTAGTTTCTGACTTCCGAA
AAGATTTCCAATTCTATAAAGTACGTGAGATTAACAATTACCATCATGCCCATGATG
CGTATCTAAATGCCGTCGTTGGAACTGCTTTGATTAAGAAATATCCAAAACTTGAAT
CGGAGTTTGTCTATGGTGATTATAAAGTTTATGATGTTCGTAAAATGATTGCTAAGT
CTGAGCAAGAAATAGGCAAAGCAACCGCAAAATATTTCTTTTACTCTAATATCATGA
ACTTCTTCAAAACAGAAATTACACTTGCAAATGGAGAGATTCGCAAACGCCCTCTAA
TCGAAACTAATGGGGAAACTGGAGAAATTGTCTGGGATAAAGGGCGAGATTTTGCCA
CAGTGCGCAAAGTATTGTCCATGCCCCAAGTCAATATTGTCAAGAAAACAGAAGTAC
AGACAGGCGGATTCTCCAAGGAGTCAATTTTACCAAAAAGAAATTCGGACAAGCTTA
TTGCTCGTAAAAAAGACTGGGATCCAAAAAAATATGGTGGTTTTgtgAGTCCAACGG
TAGCTTATTCAGTCCTAGTGGTTGCTAAGGTGGAAAAAGGGAAATCGAAGAAGTTAA
AATCCGTTAAAGAGTTACTAGGGATCACAATTATGGAAAGAAGTTCCTTTGAAAAAA
ATCCGATTGACTTTTTAGAAGCTAAAGGATATAAGGAAGTTAAAAAAGACTTAATCA
TTAAACTACCTAAATATAGTCTTTTTGAGTTAGAAAACGGTCGTAAACGGATGCTGG
CTAGTGCCGGAGAATTACAAAAAGGAAATGAGCTGGCTCTGCCAAGCAAATATGTGA
ATTTTTTATATTTAGCTAGTCATTATGAAAAGTTGAAGGGTAGTCCAGAAGATAACG
AACAAAAACAATTGTTTGTGGAGCAGCATAAGCATTATTTAGATGAGATTATTGAGC
AAATCAGTGAATTTTCTAAGCGTGTTATTTTAGCAGATGCCAATTTAGATAAAGTTC
TTAGTGCATATAACAAACATAGAGACAAACCAATACGTGAACAAGCAGAAAATATTA
TTCATTTATTTACGTTGACGAATCTTGGAGCTCCCGCTGCTTTTAAATATTTTGATA
CAACAATTGATCGTAAAcagTATcgtTCTACAAAAGAAGTTTTAGATGCCACTCTTA
TCCATCAATCCATCACTGGTCTTTATGAAACACGCATTGATTTGAGTCAGCTAGGAG
GTGAC
PAM Position: 3′
PAM: NGAG
Guide RNA Scaffold:
Nucleotide Sequence
(SEQ ID NO: 8)
GTTTTAGAGCTAGAAATAGCAAGTTAAAATAAGGCTAGTCCGTTATCAACTT
GAAAAAGTGGCACCGAGTCGGTGCTTTTTTT
Reference: Kleinstiver, et al. (2015) Nature 523.7561:481-5.
5. Variant Name: dSpCas9(EQR)
Variant DNA:
Nucleotide Sequence
(SEQ ID NO: 9)
ATGGATAAGAAATACTCAATAGGCTTAGCTATCGGCACAAATAGCGTCGGAT
GGGCGGTGATCACTGATGAATATAAGGTTCCGTCTAAAAAGTTCAAGGTTCTGGGAA
ATACAGACCGCCACAGTATCAAAAAAAATCTTATAGGGGCTCTTTTATTTGACAGTG
GAGAGACAGCGGAAGCGACTCGTCTCAAACGGACAGCTCGTAGAAGGTATACACGTC
GGAAGAATCGTATTTGTTATCTACAGGAGATTTTTTCAAATGAGATGGCGAAAGTAG
ATGATAGTTTCTTTCATCGACTTGAAGAGTCTTTTTTGGTGGAAGAAGACAAGAAGC
ATGAACGTCATCCTATTTTTGGAAATATAGTAGATGAAGTTGCTTATCATGAGAAAT
ATCCAACTATCTATCATCTGCGAAAAAAATTGGTAGATTCTACTGATAAAGCGGATT
TGCGCTTAATCTATTTGGCCTTAGCGCATATGATTAAGTTTCGTGGTCATTTTTTGA
TTGAGGGAGATTTAAATCCTGATAATAGTGATGTGGACAAACTATTTATCCAGTTGG
TACAAACCTACAATCAATTATTTGAAGAAAACCCTATTAACGCAAGTGGAGTAGATG
CTAAAGCGATTCTTTCTGCACGATTGAGTAAATCAAGACGATTAGAAAATCTCATTG
CTCAGCTCCCCGGTGAGAAGAAAAATGGCTTATTTGGGAATCTCATTGCTTTGTCAT
TGGGTTTGACCCCTAATTTTAAATCAAATTTTGATTTGGCAGAAGATGCTAAATTAC
AGCTTTCAAAAGATACTTACGATGATGATTTAGATAATTTATTGGCGCAAATTGGAG
ATCAATATGCTGATTTGTTTTTGGCAGCTAAGAATTTATCAGATGCTATTTTACTTT
CAGATATCCTAAGAGTAAATACTGAAATAACTAAGGCTCCCCTATCAGCTTCAATGA
TTAAACGCTACGATGAACATCATCAAGACTTGACTCTTTTAAAAGCTTTAGTTCGAC
AACAACTTCCAGAAAAGTATAAAGAAATCTTTTTTGATCAATCAAAAAACGGATATG
CAGGTTATATTGATGGGGGAGCTAGCCAAGAAGAATTTTATAAATTTATCAAACCAA
TTTTAGAAAAAATGGATGGTACTGAGGAATTATTGGTGAAACTAAATCGTGAAGATT
TGCTGCGCAAGCAACGGACCTTTGACAACGGCTCTATTCCCCATCAAATTCACTTGG
GTGAGCTGCATGCTATTTTGAGAAGACAAGAAGACTTTTATCCATTTTTAAAAGACA
ATCGTGAGAAGATTGAAAAAATCTTGACTTTTCGAATTCCTTATTATGTTGGTCCAT
TGGCGCGTGGCAATAGTCGTTTTGCATGGATGACTCGGAAGTCTGAAGAAACAATTA
CCCCATGGAATTTTGAAGAAGTTGTCGATAAAGGTGCTTCAGCTCAATCATTTATTG
AACGCATGACAAACTTTGATAAAAATCTTCCAAATGAAAAAGTACTACCAAAACATA
GTTTGCTTTATGAGTATTTTACGGTTTATAACGAATTGACAAAGGTCAAATATGTTA
CTGAAGGAATGCGAAAACCAGCATTTCTTTCAGGTGAACAGAAGAAAGCCATTGTTG
ATTTACTCTTCAAAACAAATCGAAAAGTAACCGTTAAGCAATTAAAAGAAGATTATT
TCAAAAAAATAGAATGTTTTGATAGTGTTGAAATTTCAGGAGTTGAAGATAGATTTA
ATGCTTCATTAGGTACCTACCATGATTTGCTAAAAATTATTAAAGATAAAGATTTTT
TGGATAATGAAGAAAATGAAGATATCTTAGAGGATATTGTTTTAACATTGACCTTAT
TTGAAGATAGGGAGATGATTGAGGAAAGACTTAAAACATATGCTCACCTCTTTGATG
ATAAGGTGATGAAACAGCTTAAACGTCGCCGTTATACTGGTTGGGGACGTTTGTCTC
GAAAATTGATTAATGGTATTAGGGATAAGCAATCTGGCAAAACAATATTAGATTTTT
TGAAATCAGATGGTTTTGCCAATCGCAATTTTATGCAGCTGATCCATGATGATAGTT
TGACATTTAAAGAAGACATTCAAAAAGCACAAGTGTCTGGACAAGGCGATAGTTTAC
ATGAACATATTGCAAATTTAGCTGGTAGCCCTGCTATTAAAAAAGGTATTTTACAGA
CTGTAAAAGTTGTTGATGAATTGGTCAAAGTAATGGGGCGGCATAAGCCAGAAAATA
TCGTTATTGAAATGGCACGTGAAAATCAGACAACTCAAAAGGGCCAGAAAAATTCGC
GAGAGCGTATGAAACGAATCGAAGAAGGTATCAAAGAATTAGGAAGTCAGATTCTTA
AAGAGCATCCTGTTGAAAATACTCAATTGCAAAATGAAAAGCTCTATCTCTATTATC
TCCAAAATGGAAGAGACATGTATGTGGACCAAGAATTAGATATTAATCGTTTAAGTG
ATTATGATGTCGATGCCATTGTTCCACAAAGTTTCCTTAAAGACGATTCAATAGACA
ATAAGGTCTTAACGCGTTCTGATAAAAATCGTGGTAAATCGGATAACGTTCCAAGTG
AAGAAGTAGTCAAAAAGATGAAAAACTATTGGAGACAACTTCTAAACGCCAAGTTAA
TCACTCAACGTAAGTTTGATAATTTAACGAAAGCTGAACGTGGAGGTTTGAGTGAAC
TTGATAAAGCTGGTTTTATCAAACGCCAATTGGTTGAAACTCGCCAAATCACTAAGC
ATGTGGCACAAATTTTGGATAGTCGCATGAATACTAAATACGATGAAAATGATAAAC
TTATTCGAGAGGTTAAAGTGATTACCTTAAAATCTAAATTAGTTTCTGACTTCCGAA
AAGATTTCCAATTCTATAAAGTACGTGAGATTAACAATTACCATCATGCCCATGATG
CGTATCTAAATGCCGTCGTTGGAACTGCTTTGATTAAGAAATATCCAAAACTTGAAT
CGGAGTTTGTCTATGGTGATTATAAAGTTTATGATGTTCGTAAAATGATTGCTAAGT
CTGAGCAAGAAATAGGCAAAGCAACCGCAAAATATTTCTTTTACTCTAATATCATGA
ACTTCTTCAAAACAGAAATTACACTTGCAAATGGAGAGATTCGCAAACGCCCTCTAA
TCGAAACTAATGGGGAAACTGGAGAAATTGTCTGGGATAAAGGGCGAGATTTTGCCA
CAGTGCGCAAAGTATTGTCCATGCCCCAAGTCAATATTGTCAAGAAAACAGAAGTAC
AGACAGGCGGATTCTCCAAGGAGTCAATTTTACCAAAAAGAAATTCGGACAAGCTTA
TTGCTCGTAAAAAAGACTGGGATCCAAAAAAATATGGTGGTTTTGAAAGTCCAACGG
TAGCTTATTCAGTCCTAGTGGTTGCTAAGGTGGAAAAAGGGAAATCGAAGAAGTTAA
AATCCGTTAAAGAGTTACTAGGGATCACAATTATGGAAAGAAGTTCCTTTGAAAAAA
ATCCGATTGACTTTTTAGAAGCTAAAGGATATAAGGAAGTTAAAAAAGACTTAATCA
TTAAACTACCTAAATATAGTCTTTTTGAGTTAGAAAACGGTCGTAAACGGATGCTGG
CTAGTGCCGGAGAATTACAAAAAGGAAATGAGCTGGCTCTGCCAAGCAAATATGTGA
ATTTTTTATATTTAGCTAGTCATTATGAAAAGTTGAAGGGTAGTCCAGAAGATAACG
AACAAAAACAATTGTTTGTGGAGCAGCATAAGCATTATTTAGATGAGATTATTGAGC
AAATCAGTGAATTTTCTAAGCGTGTTATTTTAGCAGATGCCAATTTAGATAAAGTTC
TTAGTGCATATAACAAACATAGAGACAAACCAATACGTGAACAAGCAGAAAATATTA
TTCATTTATTTACGTTGACGAATCTTGGAGCTCCCGCTGCTTTTAAATATTTTGATA
CAACAATTGATCGTAAACAGTATCGTTCTACAAAAGAAGTTTTAGATGCCACTCTTA
TCCATCAATCCATCACTGGTCTTTATGAAACACGCATTGATTTGAGTCAGCTAGGAG
GTGAC
PAM Position: 3′
PAM: NGAG
Guide RNA Scaffold:
Nucleotide Sequence
(SEQ ID NO: 10)
GTTTTAGAGCTAGAAATAGCAAGTTAAAATAAGGCTAGTCCGTTATCAACTT
GAAAAAGTGGCACCGAGTCGGTGCTTTTTTT
Reference: Kleinstiver, et al. (2015) Nature 523.7561:481-5.
6. Variant Name: desSpCas9
Variant DNA:
Nucleotide Sequence
(SEQ ID NO: 11)
ATGGATAAGAAATACTCAATAGGCTTAGCTATCGGCACAAATAGCGTCGGAT
GGGCGGTGATCACTGATGAATATAAGGTTCCGTCTAAAAAGTTCAAGGTTCTGGGAA
ATACAGACCGCCACAGTATCAAAAAAAATCTTATAGGGGCTCTTTTATTTGACAGTG
GAGAGACAGCGGAAGCGACTCGTCTCAAACGGACAGCTCGTAGAAGGTATACACGTC
GGAAGAATCGTATTTGTTATCTACAGGAGATTTTTTCAAATGAGATGGCGAAAGTAG
ATGATAGTTTCTTTCATCGACTTGAAGAGTCTTTTTTGGTGGAAGAAGACAAGAAGC
ATGAACGTCATCCTATTTTTGGAAATATAGTAGATGAAGTTGCTTATCATGAGAAAT
ATCCAACTATCTATCATCTGCGAAAAAAATTGGTAGATTCTACTGATAAAGCGGATT
TGCGCTTAATCTATTTGGCCTTAGCGCATATGATTAAGTTTCGTGGTCATTTTTTGA
TTGAGGGAGATTTAAATCCTGATAATAGTGATGTGGACAAACTATTTATCCAGTTGG
TACAAACCTACAATCAATTATTTGAAGAAAACCCTATTAACGCAAGTGGAGTAGATG
CTAAAGCGATTCTTTCTGCACGATTGAGTAAATCAAGACGATTAGAAAATCTCATTG
CTCAGCTCCCCGGTGAGAAGAAAAATGGCTTATTTGGGAATCTCATTGCTTTGTCAT
TGGGTTTGACCCCTAATTTTAAATCAAATTTTGATTTGGCAGAAGATGCTAAATTAC
AGCTTTCAAAAGATACTTACGATGATGATTTAGATAATTTATTGGCGCAAATTGGAG
ATCAATATGCTGATTTGTTTTTGGCAGCTAAGAATTTATCAGATGCTATTTTACTTT
CAGATATCCTAAGAGTAAATACTGAAATAACTAAGGCTCCCCTATCAGCTTCAATGA
TTAAACGCTACGATGAACATCATCAAGACTTGACTCTTTTAAAAGCTTTAGTTCGAC
AACAACTTCCAGAAAAGTATAAAGAAATCTTTTTTGATCAATCAAAAAACGGATATG
CAGGTTATATTGATGGGGGAGCTAGCCAAGAAGAATTTTATAAATTTATCAAACCAA
TTTTAGAAAAAATGGATGGTACTGAGGAATTATTGGTGAAACTAAATCGTGAAGATT
TGCTGCGCAAGCAACGGACCTTTGACAACGGCTCTATTCCCCATCAAATTCACTTGG
GTGAGCTGCATGCTATTTTGAGAAGACAAGAAGACTTTTATCCATTTTTAAAAGACA
ATCGTGAGAAGATTGAAAAAATCTTGACTTTTCGAATTCCTTATTATGTTGGTCCAT
TGGCGCGTGGCAATAGTCGTTTTGCATGGATGACTCGGAAGTCTGAAGAAACAATTA
CCCCATGGAATTTTGAAGAAGTTGTCGATAAAGGTGCTTCAGCTCAATCATTTATTG
AACGCATGACAAACTTTGATAAAAATCTTCCAAATGAAAAAGTACTACCAAAACATA
GTTTGCTTTATGAGTATTTTACGGTTTATAACGAATTGACAAAGGTCAAATATGTTA
CTGAAGGAATGCGAAAACCAGCATTTCTTTCAGGTGAACAGAAGAAAGCCATTGTTG
ATTTACTCTTCAAAACAAATCGAAAAGTAACCGTTAAGCAATTAAAAGAAGATTATT
TCAAAAAAATAGAATGTTTTGATAGTGTTGAAATTTCAGGAGTTGAAGATAGATTTA
ATGCTTCATTAGGTACCTACCATGATTTGCTAAAAATTATTAAAGATAAAGATTTTT
TGGATAATGAAGAAAATGAAGATATCTTAGAGGATATTGTTTTAACATTGACCTTAT
TTGAAGATAGGGAGATGATTGAGGAAAGACTTAAAACATATGCTCACCTCTTTGATG
ATAAGGTGATGAAACAGCTTAAACGTCGCCGTTATACTGGTTGGGGACGTTTGTCTC
GAAAATTGATTAATGGTATTAGGGATAAGCAATCTGGCAAAACAATATTAGATTTTT
TGAAATCAGATGGTTTTGCCAATCGCAATTTTATGCAGCTGATCCATGATGATAGTT
TGACATTTAAAGAAGACATTCAAAAAGCACAAGTGTCTGGACAAGGCGATAGTTTAC
ATGAACATATTGCAAATTTAGCTGGTAGCCCTGCTATTAAAAAAGGTATTTTACAGA
CTGTAAAAGTTGTTGATGAATTGGTCAAAGTAATGGGGCGGCATAAGCCAGAAAATA
TCGTTATTGAAATGGCACGTGAAAATCAGACAACTCAAAAGGGCCAGAAAAATTCGC
GAGAGCGTATGAAACGAATCGAAGAAGGTATCAAAGAATTAGGAAGTCAGATTCTTA
AAGAGCATCCTGTTGAAAATACTCAATTGCAAAATGAAAAGCTCTATCTCTATTATC
TCCAAAATGGAAGAGACATGTATGTGGACCAAGAATTAGATATTAATCGTTTAAGTG
ATTATGATGTCGATGCCATTGTTCCACAAAGTTTCCTTGCTGACGATTCAATAGACA
ATAAGGTCTTAACGCGTTCTGATAAAAATCGTGGTAAATCGGATAACGTTCCAAGTG
AAGAAGTAGTCAAAAAGATGAAAAACTATTGGAGACAACTTCTAAACGCCAAGTTAA
TCACTCAACGTAAGTTTGATAATTTAACGAAAGCTGAACGTGGAGGTTTGAGTGAAC
TTGATAAAGCTGGTTTTATCAAACGCCAATTGGTTGAAACTCGCCAAATCACTAAGC
ATGTGGCACAAATTTTGGATAGTCGCATGAATACTAAATACGATGAAAATGATAAAC
TTATTCGAGAGGTTAAAGTGATTACCTTAAAATCTAAATTAGTTTCTGACTTCCGAA
AAGATTTCCAATTCTATAAAGTACGTGAGATTAACAATTACCATCATGCCCATGATG
CGTATCTAAATGCCGTCGTTGGAACTGCTTTGATTAAGAAATATCCAGCTCTTGAAT
CGGAGTTTGTCTATGGTGATTATAAAGTTTATGATGTTCGTAAAATGATTGCTAAGT
CTGAGCAAGAAATAGGCAAAGCAACCGCAAAATATTTCTTTTACTCTAATATCATGA
ACTTCTTCAAAACAGAAATTACACTTGCAAATGGAGAGATTCGCAAAGCTCCTCTAA
TCGAAACTAATGGGGAAACTGGAGAAATTGTCTGGGATAAAGGGCGAGATTTTGCCA
CAGTGCGCAAAGTATTGTCCATGCCCCAAGTCAATATTGTCAAGAAAACAGAAGTAC
AGACAGGCGGATTCTCCAAGGAGTCAATTTTACCAAAAAGAAATTCGGACAAGCTTA
TTGCTCGTAAAAAAGACTGGGATCCAAAAAAATATGGTGGTTTTGATAGTCCAACGG
TAGCTTATTCAGTCCTAGTGGTTGCTAAGGTGGAAAAAGGGAAATCGAAGAAGTTAA
AATCCGTTAAAGAGTTACTAGGGATCACAATTATGGAAAGAAGTTCCTTTGAAAAAA
ATCCGATTGACTTTTTAGAAGCTAAAGGATATAAGGAAGTTAAAAAAGACTTAATCA
TTAAACTACCTAAATATAGTCTTTTTGAGTTAGAAAACGGTCGTAAACGGATGCTGG
CTAGTGCCGGAGAATTACAAAAAGGAAATGAGCTGGCTCTGCCAAGCAAATATGTGA
ATTTTTTATATTTAGCTAGTCATTATGAAAAGTTGAAGGGTAGTCCAGAAGATAACG
AACAAAAACAATTGTTTGTGGAGCAGCATAAGCATTATTTAGATGAGATTATTGAGC
AAATCAGTGAATTTTCTAAGCGTGTTATTTTAGCAGATGCCAATTTAGATAAAGTTC
TTAGTGCATATAACAAACATAGAGACAAACCAATACGTGAACAAGCAGAAAATATTA
TTCATTTATTTACGTTGACGAATCTTGGAGCTCCCGCTGCTTTTAAATATTTTGATA
CAACAATTGATCGTAAACGATATACGTCTACAAAAGAAGTTTTAGATGCCACTCTTA
TCCATCAATCCATCACTGGTCTTTATGAAACACGCATTGATTTGAGTCAGCTAGGAG
GTGAC
PAM Position: 3′
PAM: NGG
Guide RNA Scaffold:
Nucleotide Sequence
(SEQ ID NO: 12)
GTTTTAGAGCTAGAAATAGCAAGTTAAAATAAGGCTAGTCCGTTATCAACTT
GAAAAAGTGGCACCGAGTCGGTGCTTTTTTT
Reference: Slaymaker, et al. (2016) Science 351.6268:84-8.
7. Variant Name: dSpCas9-HF1
Variant DNA:
Nucleotide Sequence
(SEQ ID NO: 13)
ATGGATAAGAAATACTCAATAGGCTTAGCTATCGGCACAAATAGCGTCGGAT
GGGCGGTGATCACTGATGAATATAAGGTTCCGTCTAAAAAGTTCAAGGTTCTGGGAA
ATACAGACCGCCACAGTATCAAAAAAAATCTTATAGGGGCTCTTTTATTTGACAGTG
GAGAGACAGCGGAAGCGACTCGTCTCAAACGGACAGCTCGTAGAAGGTATACACGTC
GGAAGAATCGTATTTGTTATCTACAGGAGATTTTTTCAAATGAGATGGCGAAAGTAG
ATGATAGTTTCTTTCATCGACTTGAAGAGTCTTTTTTGGTGGAAGAAGACAAGAAGC
ATGAACGTCATCCTATTTTTGGAAATATAGTAGATGAAGTTGCTTATCATGAGAAAT
ATCCAACTATCTATCATCTGCGAAAAAAATTGGTAGATTCTACTGATAAAGCGGATT
TGCGCTTAATCTATTTGGCCTTAGCGCATATGATTAAGTTTCGTGGTCATTTTTTGA
TTGAGGGAGATTTAAATCCTGATAATAGTGATGTGGACAAACTATTTATCCAGTTGG
TACAAACCTACAATCAATTATTTGAAGAAAACCCTATTAACGCAAGTGGAGTAGATG
CTAAAGCGATTCTTTCTGCACGATTGAGTAAATCAAGACGATTAGAAAATCTCATTG
CTCAGCTCCCCGGTGAGAAGAAAAATGGCTTATTTGGGAATCTCATTGCTTTGTCAT
TGGGTTTGACCCCTAATTTTAAATCAAATTTTGATTTGGCAGAAGATGCTAAATTAC
AGCTTTCAAAAGATACTTACGATGATGATTTAGATAATTTATTGGCGCAAATTGGAG
ATCAATATGCTGATTTGTTTTTGGCAGCTAAGAATTTATCAGATGCTATTTTACTTT
CAGATATCCTAAGAGTAAATACTGAAATAACTAAGGCTCCCCTATCAGCTTCAATGA
TTAAACGCTACGATGAACATCATCAAGACTTGACTCTTTTAAAAGCTTTAGTTCGAC
AACAACTTCCAGAAAAGTATAAAGAAATCTTTTTTGATCAATCAAAAAACGGATATG
CAGGTTATATTGATGGGGGAGCTAGCCAAGAAGAATTTTATAAATTTATCAAACCAA
TTTTAGAAAAAATGGATGGTACTGAGGAATTATTGGTGAAACTAAATCGTGAAGATT
TGCTGCGCAAGCAACGGACCTTTGACAACGGCTCTATTCCCCATCAAATTCACTTGG
GTGAGCTGCATGCTATTTTGAGAAGACAAGAAGACTTTTATCCATTTTTAAAAGACA
ATCGTGAGAAGATTGAAAAAATCTTGACTTTTCGAATTCCTTATTATGTTGGTCCAT
TGGCGCGTGGCAATAGTCGTTTTGCATGGATGACTCGGAAGTCTGAAGAAACAATTA
CCCCATGGAATTTTGAAGAAGTTGTCGATAAAGGTGCTTCAGCTCAATCATTTATTG
AACGCATGACAGCTTTTGATAAAAATCTTCCAAATGAAAAAGTACTACCAAAACATA
GTTTGCTTTATGAGTATTTTACGGTTTATAACGAATTGACAAAGGTCAAATATGTTA
CTGAAGGAATGCGAAAACCAGCATTTCTTTCAGGTGAACAGAAGAAAGCCATTGTTG
ATTTACTCTTCAAAACAAATCGAAAAGTAACCGTTAAGCAATTAAAAGAAGATTATT
TCAAAAAAATAGAATGTTTTGATAGTGTTGAAATTTCAGGAGTTGAAGATAGATTTA
ATGCTTCATTAGGTACCTACCATGATTTGCTAAAAATTATTAAAGATAAAGATTTTT
TGGATAATGAAGAAAATGAAGATATCTTAGAGGATATTGTTTTAACATTGACCTTAT
TTGAAGATAGGGAGATGATTGAGGAAAGACTTAAAACATATGCTCACCTCTTTGATG
ATAAGGTGATGAAACAGCTTAAACGTCGCCGTTATACTGGTTGGGGAGCTTTGTCTC
GAAAATTGATTAATGGTATTAGGGATAAGCAATCTGGCAAAACAATATTAGATTTTT
TGAAATCAGATGGTTTTGCCAATCGCAATTTTATGGCTCTGATCCATGATGATAGTT
TGACATTTAAAGAAGACATTCAAAAAGCACAAGTGTCTGGACAAGGCGATAGTTTAC
ATGAACATATTGCAAATTTAGCTGGTAGCCCTGCTATTAAAAAAGGTATTTTACAGA
CTGTAAAAGTTGTTGATGAATTGGTCAAAGTAATGGGGCGGCATAAGCCAGAAAATA
TCGTTATTGAAATGGCACGTGAAAATCAGACAACTCAAAAGGGCCAGAAAAATTCGC
GAGAGCGTATGAAACGAATCGAAGAAGGTATCAAAGAATTAGGAAGTCAGATTCTTA
AAGAGCATCCTGTTGAAAATACTCAATTGCAAAATGAAAAGCTCTATCTCTATTATC
TCCAAAATGGAAGAGACATGTATGTGGACCAAGAATTAGATATTAATCGTTTAAGTG
ATTATGATGTCGATGCCATTGTTCCACAAAGTTTCCTTAAAGACGATTCAATAGACA
ATAAGGTCTTAACGCGTTCTGATAAAAATCGTGGTAAATCGGATAACGTTCCAAGTG
AAGAAGTAGTCAAAAAGATGAAAAACTATTGGAGACAACTTCTAAACGCCAAGTTAA
TCACTCAACGTAAGTTTGATAATTTAACGAAAGCTGAACGTGGAGGTTTGAGTGAAC
TTGATAAAGCTGGTTTTATCAAACGCCAATTGGTTGAAACTCGCGCTATCACTAAGC
ATGTGGCACAAATTTTGGATAGTCGCATGAATACTAAATACGATGAAAATGATAAAC
TTATTCGAGAGGTTAAAGTGATTACCTTAAAATCTAAATTAGTTTCTGACTTCCGAA
AAGATTTCCAATTCTATAAAGTACGTGAGATTAACAATTACCATCATGCCCATGATG
CGTATCTAAATGCCGTCGTTGGAACTGCTTTGATTAAGAAATATCCAAAACTTGAAT
CGGAGTTTGTCTATGGTGATTATAAAGTTTATGATGTTCGTAAAATGATTGCTAAGT
CTGAGCAAGAAATAGGCAAAGCAACCGCAAAATATTTCTTTTACTCTAATATCATGA
ACTTCTTCAAAACAGAAATTACACTTGCAAATGGAGAGATTCGCAAACGCCCTCTAA
TCGAAACTAATGGGGAAACTGGAGAAATTGTCTGGGATAAAGGGCGAGATTTTGCCA
CAGTGCGCAAAGTATTGTCCATGCCCCAAGTCAATATTGTCAAGAAAACAGAAGTAC
AGACAGGCGGATTCTCCAAGGAGTCAATTTTACCAAAAAGAAATTCGGACAAGCTTA
TTGCTCGTAAAAAAGACTGGGATCCAAAAAAATATGGTGGTTTTGATAGTCCAACGG
TAGCTTATTCAGTCCTAGTGGTTGCTAAGGTGGAAAAAGGGAAATCGAAGAAGTTAA
AATCCGTTAAAGAGTTACTAGGGATCACAATTATGGAAAGAAGTTCCTTTGAAAAAA
ATCCGATTGACTTTTTAGAAGCTAAAGGATATAAGGAAGTTAAAAAAGACTTAATCA
TTAAACTACCTAAATATAGTCTTTTTGAGTTAGAAAACGGTCGTAAACGGATGCTGG
CTAGTGCCGGAGAATTACAAAAAGGAAATGAGCTGGCTCTGCCAAGCAAATATGTGA
ATTTTTTATATTTAGCTAGTCATTATGAAAAGTTGAAGGGTAGTCCAGAAGATAACG
AACAAAAACAATTGTTTGTGGAGCAGCATAAGCATTATTTAGATGAGATTATTGAGC
AAATCAGTGAATTTTCTAAGCGTGTTATTTTAGCAGATGCCAATTTAGATAAAGTTC
TTAGTGCATATAACAAACATAGAGACAAACCAATACGTGAACAAGCAGAAAATATTA
TTCATTTATTTACGTTGACGAATCTTGGAGCTCCCGCTGCTTTTAAATATTTTGATA
CAACAATTGATCGTAAACGATATACGTCTACAAAAGAAGTTTTAGATGCCACTCTTA
TCCATCAATCCATCACTGGTCTTTATGAAACACGCATTGATTTGAGTCAGCTAGGAG
GTGAC
PAM Position: 3′
PAM: NGG
Guide RNA Scaffold:
Nucleotide Sequence
(SEQ ID NO: 14)
GTTTTAGAGCTAGAAATAGCAAGTTAAAATAAGGCTAGTCCGTTATCAACTT
GAAAAAGTGGCACCGAGTCGGTGCTTTTTTT
Reference: Kleinstiver, et al. (2016) Nature 529(7587):490-5.
8. Variant Name: dSaCas9
Variant DNA:
Nucleotide Sequence
(SEQ ID NO: 15)
ATGAAACGTAATTATATCTTAGGTCTGGCGATTGGTATTACTAGTGTTGGCT
ATGGTATTATTGATTACGAGACGCGCGATGTCATCGACGCTGGGGTGCGTCTGTTTA
AGGAAGCCAATGTGGAAAACAATGAGGGTCGTCGTAGTAAACGTGGCGCCCGTCGTC
TGAAACGCCGTCGTCGTCATCGCATTCAGCGTGTGAAAAAACTCCTGTTTGATTATA
ACCTCCTGACCGATCACTCAGAACTCTCGGGCATCAACCCGTATGAAGCGCGTGTTA
AAGGGCTGTCTCAGAAATTATCAGAGGAAGAATTTTCGGCCGCGCTGCTGCATTTAG
CCAAACGCCGTGGGGTACACAACGTAAACGAAGTTGAAGAAGATACCGGAAACGAAC
TGTCCACTAAAGAACAAATCTCTCGCAACTCCAAAGCACTGGAGGAAAAGTATGTTG
CGGAATTGCAGCTGGAGCGTTTAAAAAAAGACGGTGAAGTACGTGGCTCCATCAATC
GCTTCAAAACCAGCGATTACGTGAAAGAGGCGAAGCAGCTGCTGAAAGTTCAAAAGG
CGTACCACCAACTGGATCAGAGCTTTATTGACACCTATATCGACCTGCTGGAAACGC
GCCGTACTTATTATGAAGGCCCTGGGGAAGGATCTCCGTTCGGCTGGAAAGACATCA
AAGAATGGTATGAAATGCTGATGGGTCATTGCACCTATTTCCCGGAGGAACTTCGTA
GTGTTAAGTACGCCTATAACGCTGATCTGTACAACGCCCTGAACGATTTGAACAACC
TGGTAATTACCCGCGATGAGAACGAAAAACTGGAGTATTACGAGAAATTCCAGATTA
TCGAAAATGTTTTCAAACAAAAAAAGAAACCGACGCTTAAGCAGATTGCGAAAGAAA
TTTTGGTTAACGAAGAAGATATTAAAGGGTACCGTGTTACTAGCACGGGTAAACCGG
AATTCACTAACCTGAAAGTGTATCATGATATCAAAGATATTACCGCCCGTAAGGAAA
TTATTGAAAACGCGGAATTACTGGACCAGATCGCCAAAATTCTCACCATCTATCAAT
CGTCCGAGGACATCCAAGAAGAGTTGACTAACCTGAATTCAGAGCTGACACAGGAAG
AGATCGAGCAGATTAGCAACTTAAAAGGTTATACCGGCACTCACAATCTTAGTCTGA
AAGCTATCAACTTGATCCTTGACGAACTGTGGCATACCAACGATAATCAGATCGCGA
TCTTTAACCGTTTAAAACTTGTGCCGAAAAAAGTGGATCTGAGCCAACAAAAAGAGA
TTCCAACCACCTTAGTGGATGATTTTATTCTCAGCCCGGTTGTTAAACGTAGCTTTA
TTCAATCAATCAAAGTGATTAACGCCATTATCAAAAAGTATGGGCTGCCAAACGACA
TTATCATCGAACTGGCTCGTGAAAAAAATTCTAAGGACGCGCAAAAAATGATTAACG
AAATGCAGAAACGCAATCGCCAGACGAATGAACGCATTGAAGAAATTATCCGTACTA
CCGGAAAAGAAAACGCCAAATACCTGATCGAAAAGATCAAACTTCACGACATGCAGG
AAGGTAAGTGTCTGTACTCCCTTGAAGCAATTCCCCTCGAGGATTTATTGAACAATC
CGTTTAATTACGAGGTGGATCATATTATCCCACGCTCTGTGAGCTTTGACAATAGCT
TTAACAATAAAGTCTTGGTGAAACAAGAAGAAGCTTCTAAAAAAGGGAACCGCACCC
CTTTCCAGTACCTGAGCAGTTCGGATAGTAAGATCTCATATGAAACTTTCAAAAAAC
ATATTCTGAACTTGGCAAAGGGCAAAGGCCGTATCTCAAAAACAAAGAAAGAATATC
TTTTGGAAGAGCGTGATATCAATCGCTTCTCAGTTCAAAAAGACTTCATCAACCGCA
ACTTAGTGGACACCCGTTATGCCACACGTGGCTTAATGAACTTACTGCGCAGCTACT
TTCGCGTGAACAATCTTGACGTGAAGGTGAAAAGCATCAACGGCGGCTTCACATCTT
TCTTGCGTCGTAAATGGAAGTTTAAAAAAGAGCGCAATAAGGGTTACAAGCATCACG
CCGAAGATGCGCTGATCATCGCGAACGCGGATTTTATCTTCAAGGAATGGAAAAAAT
TGGATAAAGCCAAAAAAGTGATGGAAAACCAAATGTTCGAAGAGAAGCAGGCAGAAA
GTATGCCGGAAATCGAAACGGAACAAGAGTACAAAGAAATCTTCATTACGCCACATC
AAATTAAGCACATTAAAGATTTCAAAGACTATAAATACTCACATCGTGTTGACAAAA
AACCGAATCGTGAATTGATTAACGATACGTTATACTCGACGCGCAAGGATGATAAAG
GGAACACATTAATCGTCAATAATTTGAACGGGCTGTACGATAAAGACAACGATAAAC
TCAAAAAGCTGATCAATAAGAGTCCGGAGAAACTGTTAATGTATCATCATGACCCCC
AGACTTATCAGAAGCTGAAATTAATCATGGAACAATACGGGGACGAAAAAAACCCAC
TTTATAAATATTATGAGGAAACAGGCAACTATCTGACAAAGTACTCTAAAAAGGATA
ATGGTCCGGTCATCAAAAAAATTAAATACTATGGTAACAAACTCAATGCGCATCTCG
ACATCACCGATGATTACCCAAATTCCCGTAACAAAGTGGTTAAACTTTCTCTGAAAC
CTTATCGCTTTGATGTATATCTGGACAATGGCGTTTACAAATTTGTTACCGTTAAAA
ACTTAGATGTTATTAAAAAAGAAAATTACTACGAAGTCAACTCAAAATGTTACGAAG
AGGCGAAAAAACTGAAAAAAATCTCCAACCAAGCGGAATTCATTGCGTCCTTTTATA
ATAATGACTTAATCAAGATTAACGGTGAGTTGTACCGCGTCATTGGAGTGAATAATG
ATCTGCTGAACCGTATCGAAGTGAACATGATCGATATTACTTATCGCGAATATCTGG
AAAATATGAACGACAAACGTCCACCTCGTATCATTAAAACCATCGCTAGTAAAACCC
AAAGCATCAAAAAATATTCCACGGATATTTTAGGTAATCTCTATGAAGTTAAATCGA
AGAAACATCCCCAGATCATTAAAAAAGGC
PAM Position: 3′
PAM: NNGRRT
Guide RNA Scaffold
Nucleotide Sequence
(SEQ ID NO: 16)
GTTTTAGTACTCTGGAAACAGAATCTACTAAAACAAGGCAAAATGCCGTGTT
TATCTCGTCAACTTGTTGGCGAGATTTTTTT
Reference: Ran, et al. (2015) Nature 520.7546:186-91
9. Variant Name: desSaCas9
Variant DNA:
Nucleotide Sequence
(SEQ ID NO: 17)
ATGAAACGTAATTATATCTTAGGTCTGGCGATTGGTATTACTAGTGTTGGCT
ATGGTATTATTGATTACGAGACGCGCGATGTCATCGACGCTGGGGTGCGTCTGTTTA
AGGAAGCCAATGTGGAAAACAATGAGGGTCGTCGTAGTAAACGTGGCGCCCGTCGTC
TGAAACGCCGTCGTCGTCATCGCATTCAGCGTGTGAAAAAACTCCTGTTTGATTATA
ACCTCCTGACCGATCACTCAGAACTCTCGGGCATCAACCCGTATGAAGCGCGTGTTA
AAGGGCTGTCTCAGAAATTATCAGAGGAAGAATTTTCGGCCGCGCTGCTGCATTTAG
CCAAACGCCGTGGGGTACACAACGTAAACGAAGTTGAAGAAGATACCGGAAACGAAC
TGTCCACTAAAGAACAAATCTCTCGCAACTCCAAAGCACTGGAGGAAAAGTATGTTG
CGGAATTGCAGCTGGAGCGTTTAAAAAAAGACGGTGAAGTACGTGGCTCCATCAATC
GCTTCAAAACCAGCGATTACGTGAAAGAGGCGAAGCAGCTGCTGAAAGTTCAAAAGG
CGTACCACCAACTGGATCAGAGCTTTATTGACACCTATATCGACCTGCTGGAAACGC
GCCGTACTTATTATGAAGGCCCTGGGGAAGGATCTCCGTTCGGCTGGAAAGACATCA
AAGAATGGTATGAAATGCTGATGGGTCATTGCACCTATTTCCCGGAGGAACTTCGTA
GTGTTAAGTACGCCTATAACGCTGATCTGTACAACGCCCTGAACGATTTGAACAACC
TGGTAATTACCCGCGATGAGAACGAAAAACTGGAGTATTACGAGAAATTCCAGATTA
TCGAAAATGTTTTCAAACAAAAAAAGAAACCGACGCTTAAGCAGATTGCGAAAGAAA
TTTTGGTTAACGAAGAAGATATTAAAGGGTACCGTGTTACTAGCACGGGTAAACCGG
AATTCACTAACCTGAAAGTGTATCATGATATCAAAGATATTACCGCCCGTAAGGAAA
TTATTGAAAACGCGGAATTACTGGACCAGATCGCCAAAATTCTCACCATCTATCAAT
CGTCCGAGGACATCCAAGAAGAGTTGACTAACCTGAATTCAGAGCTGACACAGGAAG
AGATCGAGCAGATTAGCAACTTAAAAGGTTATACCGGCACTCACAATCTTAGTCTGA
AAGCTATCAACTTGATCCTTGACGAACTGTGGCATACCAACGATAATCAGATCGCGA
TCTTTAACCGTTTAAAACTTGTGCCGAAAAAAGTGGATCTGAGCCAACAAAAAGAGA
TTCCAACCACCTTAGTGGATGATTTTATTCTCAGCCCGGTTGTTAAACGTAGCTTTA
TTCAATCAATCAAAGTGATTAACGCCATTATCAAAAAGTATGGGCTGCCAAACGACA
TTATCATCGAACTGGCTCGTGAAAAAAATTCTAAGGACGCGCAAAAAATGATTAACG
AAATGCAGAAACGCAATGCTGCGACGAATGAACGCATTGAAGAAATTATCCGTACTA
CCGGAAAAGAAAACGCCAAATACCTGATCGAAAAGATCAAACTTCACGACATGCAGG
AAGGTAAGTGTCTGTACTCCCTTGAAGCAATTCCCCTCGAGGATTTATTGAACAATC
CGTTTAATTACGAGGTGGATCATATTATCCCACGCTCTGTGAGCTTTGACAATAGCT
TTAACAATAAAGTCTTGGTGAAACAAGAAGAAGCTTCTAAAAAAGGGAACCGCACCC
CTTTCCAGTACCTGAGCAGTTCGGATAGTAAGATCTCATATGAAACTTTCAAAAAAC
ATATTCTGAACTTGGCAAAGGGCAAAGGCCGTATCTCAAAAACAAAGAAAGAATATC
TTTTGGAAGAGCGTGATATCAATCGCTTCTCAGTTCAAAAAGACTTCATCAACCGCA
ACTTAGTGGACACCCGTTATGCCACAGCTGCGTTAATGAACTTACTGCGCAGCTACT
TTCGCGTGAACAATCTTGACGTGAAGGTGAAAAGCATCAACGGCGGCTTCACATCTT
TCTTGCGTCGTAAATGGAAGTTTAAAAAAGAGCGCAATAAGGGTTACAAGCATCACG
CCGAAGATGCGCTGATCATCGCGAACGCGGATTTTATCTTCAAGGAATGGAAAAAAT
TGGATAAAGCCAAAAAAGTGATGGAAAACCAAATGTTCGAAGAGAAGCAGGCAGAAA
GTATGCCGGAAATCGAAACGGAACAAGAGTACAAAGAAATCTTCATTACGCCACATC
AAATTAAGCACATTAAAGATTTCAAAGACTATAAATACTCACATCGTGTTGACAAAA
AACCGAATCGTGAATTGATTAACGATACGTTATACTCGACGCGCAAGGATGATAAAG
GGAACACATTAATCGTCAATAATTTGAACGGGCTGTACGATAAAGACAACGATAAAC
TCAAAAAGCTGATCAATAAGAGTCCGGAGAAACTGTTAATGTATCATCATGACCCCC
AGACTTATCAGAAGCTGAAATTAATCATGGAACAATACGGGGACGAAAAAAACCCAC
TTTATAAATATTATGAGGAAACAGGCAACTATCTGACAAAGTACTCTAAAAAGGATA
ATGGTCCGGTCATCAAAAAAATTAAATACTATGGTAACAAACTCAATGCGCATCTCG
ACATCACCGATGATTACCCAAATTCCCGTAACAAAGTGGTTAAACTTTCTCTGAAAC
CTTATCGCTTTGATGTATATCTGGACAATGGCGTTTACAAATTTGTTACCGTTAAAA
ACTTAGATGTTATTAAAAAAGAAAATTACTACGAAGTCAACTCAAAATGTTACGAAG
AGGCGAAAAAACTGAAAAAAATCTCCAACCAAGCGGAATTCATTGCGTCCTTTTATA
ATAATGACTTAATCAAGATTAACGGTGAGTTGTACCGCGTCATTGGAGTGAATAATG
ATCTGCTGAACCGTATCGAAGTGAACATGATCGATATTACTTATCGCGAATATCTGG
AAAATATGAACGACAAACGTCCACCTCGTATCATTAAAACCATCGCTAGTAAAACCC
AAAGCATCAAAAAATATTCCACGGATATTTTAGGTAATCTCTATGAAGTTAAATCGA
AGAAACATCCCCAGATCATTAAAAAAGGC
PAM Position: 3′
PAM: NNGRRT
Guide RNA Scaffold:
Nucleotide Sequence
(SEQ ID NO: 18)
GTTTTAGTACTCTGGAAACAGAATCTACTAAAACAAGGCAAAATGCCGTGTT
TATCTCGTCAACTTGTTGGCGAGATTTTTTT
Reference: Slaymaker, et al. (2016) Science 351.6268:84-8.
10. Variant Name: dSt1Cas9
Variant DNA:
Nucleotide Sequence
(SEQ ID NO: 19)
ATGAGTGACCTTGTGTTGGGCCTGGCCATCGGCATTGGTTCGGTCGGAGTTG
GAATTCTGAACAAGGTCACCGGGGAAATTATTCATAAAAATTCACGTATTTTCCCGG
CGGCACAAGCGGAAAACAACTTAGTCCGCCGCACTAACCGCCAGGGCCGCCGTCTGG
CGCGTCGTAAAAAACATCGCCGCGTCCGTCTGAATCGCTTGTTTGAGGAGAGCGGTT
TGATTACTGACTTTACGAAAATCTCCATTAATCTCAACCCGTATCAGCTCCGTGTGA
AGGGCCTCACGGACGAACTGAGCAACGAGGAATTATTCATCGCACTGAAAAACATGG
TGAAACATCGCGGTATTAGTTATCTGGACGATGCCAGCGACGATGGCAACAGTAGCG
TTGGTGACTATGCTCAAATCGTGAAAGAGAACAGTAAACAACTGGAAACTAAAACCC
CGGGTCAAATCCAGTTAGAACGTTATCAAACGTATGGTCAGCTGCGTGGCGATTTCA
CAGTGGAAAAGGATGGTAAGAAGCATCGTCTGATTAATGTGTTCCCTACCTCGGCAT
ATCGTTCCGAGGCCTTGCGTATCCTGCAGACTCAGCAAGAATTTAATCCGCAGATCA
CCGATGAGTTCATCAACCGCTACCTTGAAATTCTTACCGGCAAACGCAAGTATTATC
ACGGTCCGGGTAATGAAAAAAGCCGCACGGACTACGGTCGTTATCGCACATCAGGCG
AAACTTTGGACAACATTTTTGGCATTTTAATTGGTAAATGTACGTTCTACCCGGATG
AGTTCCGCGCGGCGAAAGCGTCCTATACAGCGCAAGAGTTTAATCTGTTAAATGATT
TGAATAATCTGACAGTACCGACTGAGACGAAAAAACTTTCTAAAGAACAGAAAAATC
AGATCATTAATTACGTAAAGAACGAGAAAGCGATGGGCCCGGCGAAACTGTTTAAAT
ACATTGCCAAACTTCTGTCATGCGATGTGGCAGATATTAAAGGCTACCGCATTGATA
AAAGCGGCAAAGCTGAAATTCATACGTTTGAGGCCTATCGCAAAATGAAAACTTTAG
AAACGCTTGACATTGAACAGATGGATCGCGAAACGCTGGATAAATTGGCCTACGTAC
TCACTCTGAATACGGAACGCGAAGGCATCCAGGAAGCGCTTGAGCATGAGTTTGCAG
ATGGCTCGTTTTCCCAAAAACAAGTTGACGAATTGGTCCAATTCCGCAAAGCCAATT
CCTCCATTTTTGGTAAAGGTTGGCATAACTTTAGTGTGAAGTTAATGATGGAGTTGA
TCCCCGAACTCTACGAAACCAGTGAGGAACAAATGACCATCTTGACGCGCCTGGGTA
AACAGAAAACGACGAGCTCGAGCAACAAAACGAAATATATTGATGAGAAGCTGTTGA
CGGAGGAGATTTATAACCCGGTTGTGGCAAAGAGCGTCCGCCAGGCCATCAAAATCG
TCAACGCTGCCATTAAAGAATACGGCGATTTTGATAACATCGTTATTGAAATGGCGC
GCGAGACTAATGAGGATGATGAAAAAAAAGCGATTCAGAAAATCCAAAAAGCCAACA
AAGATGAAAAGGATGCGGCCATGCTGAAAGCCGCAAACCAATACAATGGTAAGGCCG
AGCTTCCGCATTCTGTGTTTCATGGGCATAAGCAATTAGCGACGAAAATCCGTCTGT
GGCATCAGCAGGGCGAACGTTGCCTTTACACCGGTAAAACAATCTCAATTCATGATT
TAATTAACAATAGTAACCAGTTTGAGGTCGATGCGATTCTGCCGCTGAGTATTACTT
TTGATGACTCCCTTGCTAACAAAGTCTTGGTTTATGCGACTGCCAACCAGGAGAAAG
GTCAGCGCACCCCCTATCAGGCGCTGGATTCGATGGACGATGCATGGTCATTTCGTG
AACTGAAAGCGTTCGTGCGTGAAAGTAAAACACTGTCGAATAAAAAAAAAGAATATC
TGTTGACCGAAGAGGATATTTCCAAATTTGATGTCCGCAAAAAATTTATTGAACGTA
ACCTGGTAGATACTCGCTACGCCAGTCGCGTTGTCCTGAACGCCCTGCAAGAACATT
TCCGTGCTCATAAGATTGACACTAAAGTGTCTGTAGTGCGTGGGCAGTTTACTTCCC
AGTTACGCCGCCATTGGGGGATCGAAAAAACCCGCGATACCTATCACCATCACGCGG
TCGACGCGTTGATTATCGCTGCAAGTTCGCAGCTCAACCTGTGGAAAAAACAGAAAA
ACACGCTGGTTTCGTATTCGGAAGATCAGTTGCTGGATATTGAAACTGGTGAGCTGA
TTAGTGACGATGAATACAAAGAAAGCGTATTTAAGGCACCATACCAGCATTTTGTCG
ATACCCTGAAGTCCAAAGAATTTGAAGATTCAATCCTCTTTTCCTATCAGGTGGACA
GTAAATTTAACCGTAAAATTAGTGATGCAACAATTTATGCGACGCGTCAAGCTAAGG
TGGGCAAAGATAAAGCGGACGAAACCTACGTCCTCGGTAAGATCAAAGATATCTATA
CTCAGGATGGCTATGATGCCTTCATGAAAATCTACAAAAAAGATAAATCTAAGTTTT
TGATGTATCGTCATGACCCGCAAACCTTCGAAAAAGTCATTGAGCCGATCTTGGAGA
ACTATCCGAACAAACAAATCAACGAAAAAGGCAAAGAAGTTCCGTGCAATCCCTTCT
TAAAATATAAAGAAGAACATGGTTATATCCGCAAGTATAGCAAGAAGGGTAACGGGC
CGGAAATTAAGTCTCTGAAATACTATGACTCGAAGTTGGGCAATCATATTGACATCA
CCCCGAAAGATTCAAATAATAAAGTTGTGCTGCAGAGCGTAAGTCCGTGGCGTGCGG
ATGTCTATTTCAACAAAACAACAGGAAAATATGAAATCCTGGGCCTCAAATATGCCG
ACTTACAATTCGAAAAGGGTACCGGTACATACAAAATCTCCCAGGAAAAATACAACG
ATATTAAAAAAAAAGAAGGCGTAGATAGCGACTCTGAGTTCAAATTTACGTTATATA
AGAACGATCTGCTGTTAGTAAAAGATACCGAAACCAAGGAACAGCAGTTATTTCGTT
TCCTGTCGCGTACCATGCCCAAACAGAAACACTATGTGGAACTTAAACCATACGACA
AACAAAAATTCGAGGGTGGTGAAGCGCTCATTAAAGTTTTAGGTAATGTGGCAAATT
CCGGTCAGTGCAAAAAAGGTTTAGGTAAAAGTAATATTTCCATTTATAAAGTTCGCA
CCGATGTACTGGGGAATCAGCATATTATTAAAAATGAAGGAGACAAGCCCAAACTGG
ACTTT
PAM Position: 3′
PAM: NNARAA
Guide RNA Scaffold:
Nucleotide Sequence
(SEQ ID NO: 20)
GTTTTTGTACTCTCAAGATTTAAGTAACTGTACAACGAAACTTACACAGTTA
CTTAAATCTTGCAGAAGCTACAAAGATAAGGCTTCATGCCGAAATCAACACCCTGTC
ATTTTATGGCAGGGTGTTTTTTT
Reference: Kleinstiver, et al. (2015) Nature 523.7561:481-5.
11. Variant Name: dFnCpf1
Variant DNA:
Nucleotide Sequence
(SEQ ID NO: 21)
ATGAGCATCTACCAGGAGTTCGTCAACAAGTATTCACTGAGTAAGACACTGC
GGTTCGAGCTGATCCCACAGGGCAAGACACTGGAGAACATCAAGGCCCGAGGCCTGA
TTCTGGACGATGAGAAGCGGGCAAAAGACTATAAGAAAGCCAAGCAGATCATTGATA
AATACCACCAGTTCTTTATCGAGGAAATTCTGAGCTCCGTGTGCATCAGTGAGGATC
TGCTGCAGAATTACTCAGACGTGTACTTCAAGCTGAAGAAGAGCGACGATGACAACC
TGCAGAAGGACTTCAAGTCCGCCAAGGACACCATCAAGAAACAGATTAGCGAGTACA
TCAAGGACTCCGAAAAGTTTAAAAATCTGTTCAACCAGAATCTGATCGATGCTAAGA
AAGGCCAGGAGTCCGACCTGATCCTGTGGCTGAAACAGTCTAAGGACAATGGGATTG
AACTGTTCAAGGCTAACTCCGATATCACTGATATTGACGAGGCACTGGAAATCATCA
AGAGCTTCAAGGGATGGACCACATACTTTAAAGGCTTCCACGAGAACCGCAAGAACG
TGTACTCCAGCAACGACATTCCTACCTCCATCATCTACCGAATCGTCGATGACAATC
TGCCAAAGTTCCTGGAGAACAAGGCCAAATATGAATCTCTGAAGGACAAAGCTCCCG
AGGCAATTAATTACGAACAGATCAAGAAAGATCTGGCTGAGGAACTGACATTCGATA
TCGACTATAAGACTAGCGAGGTGAACCAGAGGGTCTTTTCCCTGGACGAGGTGTTTG
AAATCGCCAATTTCAACAATTACCTGAACCAGTCCGGCATTACTAAATTCAATACCA
TCATTGGCGGGAAGTTTGTGAACGGGGAGAATACCAAGCGCAAGGGAATTAACGAAT
ACATCAATCTGTATAGCCAGCAGATCAACGACAAAACTCTGAAGAAATACAAGATGT
CTGTGCTGTTCAAACAGATCCTGAGTGATACCGAGTCCAAGTCTTTTGTCATTGATA
AACTGGAAGATGACTCAGACGTGGTCACTACCATGCAGAGCTTTTATGAGCAGATCG
CCGCTTTCAAGACAGTGGAGGAAAAATCTATTAAGGAAACTCTGAGTCTGCTGTTCG
ATGACCTGAAAGCCCAGAAGCTGGACCTGAGTAAGATCTACTTCAAAAACGATAAGA
GTCTGACAGACCTGTCACAGCAGGTGTTTGATGACTATTCCGTGATTGGGACCGCCG
TCCTGGAGTACATTACACAGCAGATCGCTCCAAAGAACCTGGATAATCCCTCTAAGA
AAGAGCAGGAACTGATCGCTAAGAAAACCGAGAAGGCAAAATATCTGAGTCTGGAAA
CAATTAAGCTGGCACTGGAGGAGTTCAACAAGCACAGGGATATTGACAAACAGTGCC
GCTTTGAGGAAATCCTGGCCAACTTCGCAGCCATCCCCATGATTTTTGATGAGATCG
CCCAGAACAAAGACAATCTGGCTCAGATCAGTATTAAGTACCAGAACCAGGGCAAGA
AAGACCTGCTGCAGGCTTCAGCAGAAGATGACGTGAAAGCCATCAAGGATCTGCTGG
ACCAGACCAACAATCTGCTGCACAAGCTGAAAATCTTCCATATTAGTCAGTCAGAGG
ATAAGGCTAATATCCTGGATAAAGACGAACACTTCTACCTGGTGTTCGAGGAATGTT
ACTTCGAGCTGGCAAACATTGTCCCCCTGTATAACAAGATTAGGAACTACATCACAC
AGAAGCCTTACTCTGACGAGAAGTTTAAACTGAACTTCGAAAATAGTACCCTGGCCA
ACGGGTGGGATAAGAACAAGGAGCCTGACAACACAGCTATCCTGTTCATCAAGGATG
ACAAGTACTATCTGGGAGTGATGAATAAGAAAAACAATAAGATCTTCGATGACAAAG
CCATTAAGGAGAACAAAGGGGAAGGATACAAGAAAATCGTGTATAAGCTGCTGCCCG
GCGCAAATAAGATGCTGCCTAAGGTGTTCTTCAGCGCCAAGAGTATCAAATTCTACA
ACCCATCCGAGGACATCCTGCGGATTAGAAATCACTCAACACATACTAAGAACGGGA
GCCCCCAGAAGGGATATGAGAAATTTGAGTTCAACATCGAGGATTGCAGGAAGTTTA
TTGACTTCTACAAGCAGAGCATCTCCAAACACCCTGAATGGAAGGATTTTGGCTTCC
GGTTTTCCGACACACAGAGATATAACTCTATCGACGAGTTCTACCGCGAGGTGGAAA
ATCAGGGGTATAAGCTGACTTTTGAGAACATTTCTGAAAGTTACATCGACAGCGTGG
TCAATCAGGGAAAGCTGTACCTGTTCCAGATCTATAACAAAGATTTTTCAGCATACA
GCAAGGGCAGACCAAACCTGCATACACTGTACTGGAAGGCCCTGTTCGATGAGAGGA
ATCTGCAGGACGTGGTCTATAAACTGAACGGAGAGGCCGAACTGTTTTACCGGAAGC
AGTCTATTCCTAAGAAAATCACTCACCCAGCTAAGGAGGCCATCGCTAACAAGAACA
AGGACAATCCTAAGAAAGAGAGCGTGTTCGAATACGATCTGATTAAGGACAAGCGGT
TCACCGAAGATAAGTTCTTTTTCCATTGTCCAATCACCATTAACTTCAAGTCAAGCG
GCGCTAACAAGTTCAACGACGAGATCAATCTGCTGCTGAAGGAAAAAGCAAACGATG
TGCACATCCTGAGCATTGCTCGAGGAGAGCGGCATCTGGCCTACTATACCCTGGTGG
ATGGCAAAGGGAATATCATTAAGCAGGATACATTCAACATCATTGGCAATGACCGGA
TGAAAACCAACTACCACGATAAACTGGCTGCAATCGAGAAGGATAGAGACTCAGCTA
GGAAGGACTGGAAGAAAATCAACAACATTAAGGAGATGAAGGAAGGCTATCTGAGCC
AGGTGGTCCATGAGATTGCAAAGCTGGTCATCGAATACAATGCCATTGTGGTGTTCG
AGGATCTGAACTTCGGCTTTAAGAGGGGGCGCTTTAAGGTGGAAAAACAGGTCTATC
AGAAGCTGGAGAAAATGCTGATCGAAAAGCTGAATTACCTGGTGTTTAAAGATAACG
AGTTCGACAAGACCGGAGGCGTCCTGAGAGCCTACCAGCTGACAGCTCCCTTTGAAA
CTTTCAAGAAAATGGGAAAACAGACAGGCATCATCTACTATGTGCCAGCCGGATTCA
CTTCCAAGATCTGCCCCGTGACCGGCTTTGTCAACCAGCTGTACCCTAAATATGAGT
CAGTGAGCAAGTCCCAGGAATTTTTCAGCAAGTTCGATAAGATCTGTTATAATCTGG
ACAAGGGGTACTTCGAGTTTTCCTTCGATTACAAGAACTTCGGCGACAAGGCCGCTA
AGGGGAAATGGACCATTGCCTCCTTCGGATCTCGCCTGATCAACTTTCGAAATTCCG
ATAAAAACCACAATTGGGACACTAGGGAGGTGTACCCAACCAAGGAGCTGGAAAAGC
TGCTGAAAGACTACTCTATCGAGTATGGACATGGCGAATGCATCAAGGCAGCCATCT
GTGGCGAGAGTGATAAGAAATTTTTCGCCAAGCTGACCTCAGTGCTGAATACAATCC
TGCAGATGCGGAACTCAAAGACCGGGACAGAACTGGACTATCTGATTAGCCCCGTGG
CTGATGTCAACGGAAACTTCTTCGACAGCAGACAGGCACCCAAAAATATGCCTCAGG
ATGCAGACGCCAACGGGGCCTACCACATCGGGCTGAAGGGACTGATGCTGCTGGGCC
GGATCAAGAACAATCAGGAGGGGAAGAAGCTGAACCTGGTCATTAAGAACGAGGAAT
ACTTCGAGTTTGTCCAGAATAGAAATAAC
PAM Position: 5′
PAM: TTN
Guide RNA Scaffold:
Nucleotide Sequence
(SEQ ID NO: 22)
TAATTTCTACTGTTGTAGAT
Reference: Zetsche, et al. (2015) Cell 163.3:759-71.
12. Variant Name: dAsCpf1
Variant DNA:
Nucleotide Sequence
(SEQ ID NO: 23)
ATGACCCAATTTGAAGGGTTTACCAACCTTTACCAAGTTAGCAAAACCCTGC
GTTTCGAGTTGATCCCACAAGGAAAAACCCTGAAACACATTCAGGAACAGGGATTCA
TTGAAGAGGATAAAGCTCGTAACGATCACTATAAGGAACTTAAGCCGATTATCGATC
GCATTTACAAAACGTATGCGGATCAGTGCCTGCAGCTTGTGCAACTGGACTGGGAAA
ACCTGAGCGCCGCCATTGATTCTTATCGCAAAGAAAAGACCGAAGAAACCCGTAACG
CCTTGATTGAGGAACAAGCCACGTATCGCAATGCCATTCATGATTATTTCATCGGCC
GTACGGACAATTTAACGGACGCAATTAACAAACGCCACGCTGAGATCTACAAGGGCC
TGTTTAAAGCTGAACTGTTTAATGGCAAAGTGCTCAAGCAACTGGGCACCGTCACCA
CCACCGAACATGAGAACGCTCTCCTGCGCAGTTTTGATAAATTCACCACATATTTTT
CGGGGTTCTATGAAAACCGCAAAAATGTTTTCTCAGCGGAAGATATTAGCACCGCGA
TCCCTCATCGTATCGTGCAGGATAACTTCCCTAAGTTCAAAGAGAACTGTCACATCT
TTACTCGCCTGATTACCGCTGTACCCTCGTTGCGCGAACACTTTGAAAACGTCAAAA
AAGCAATCGGCATTTTTGTATCGACCAGTATTGAAGAGGTTTTCAGCTTCCCGTTCT
ATAACCAGCTGCTGACGCAAACGCAAATCGATCTGTACAATCAGCTGTTAGGTGGCA
TTTCCCGCGAAGCGGGCACCGAAAAAATCAAAGGGCTGAATGAAGTACTGAACCTGG
CGATCCAAAAAAACGACGAAACGGCGCACATTATCGCCTCTCTGCCGCATCGCTTTA
TTCCCCTGTTCAAGCAGATCCTGAGCGATCGCAACACTCTGAGTTTCATTCTCGAAG
AATTCAAATCTGACGAAGAAGTTATTCAGTCTTTCTGTAAATATAAAACCCTGCTGC
GTAACGAGAATGTACTGGAAACCGCGGAAGCACTGTTTAACGAGCTGAATTCCATTG
ATCTGACCCATATTTTTATCTCTCACAAAAAACTGGAAACCATTAGCTCTGCGCTCT
GCGATCATTGGGACACGCTTCGCAACGCGCTCTACGAGCGCCGTATTTCCGAGTTGA
CGGGGAAAATCACCAAATCCGCTAAGGAAAAAGTTCAGCGTAGCTTGAAACATGAAG
ATATTAATCTGCAGGAGATCATTAGTGCGGCTGGTAAAGAACTTTCAGAAGCGTTTA
AACAGAAAACTTCGGAAATCTTATCGCACGCACATGCGGCTCTTGATCAGCCGCTTC
CAACGACCCTGAAAAAACAGGAGGAAAAAGAAATTCTGAAAAGTCAACTGGATAGTT
TATTGGGCCTGTACCACCTGCTTGACTGGTTTGCGGTGGATGAATCCAATGAAGTCG
ATCCCGAGTTTTCCGCACGCCTGACTGGGATCAAACTGGAAATGGAACCAAGCCTGT
CTTTTTACAACAAAGCGCGTAACTACGCGACCAAAAAGCCATACAGTGTTGAAAAGT
TTAAATTGAACTTTCAGATGCCAACCCTGGCGAGTGGCTGGGATGTGAATAAAGAAA
AAAATAATGGTGCAATCCTGTTTGTTAAAAATGGACTGTACTATCTGGGTATTATGC
CGAAACAAAAAGGTCGCTATAAAGCGCTGTCATTTGAGCCGACAGAAAAGACGAGTG
AAGGCTTTGATAAAATGTACTATGATTACTTTCCAGATGCAGCGAAAATGATTCCTA
AGTGTAGTACCCAGCTGAAGGCAGTAACAGCCCACTTCCAGACCCACACCACCCCGA
TTTTGTTAAGCAACAACTTTATTGAACCGCTGGAAATTACAAAAGAAATCTATGACC
TTAACAACCCAGAGAAGGAACCCAAGAAGTTCCAAACGGCGTATGCGAAAAAGACTG
GCGATCAAAAAGGGTATCGTGAGGCGCTGTGCAAATGGATTGATTTTACGCGCGATT
TTCTGAGCAAATATACAAAAACCACCTCGATTGATCTGAGTAGCCTGCGTCCTAGTA
GTCAGTATAAGGATTTGGGCGAATACTATGCGGAACTTAATCCGCTGCTGTACCATA
TTTCGTTCCAGCGTATCGCGGAAAAAGAGATTATGGATGCGGTAGAAACCGGGAAAT
TGTATCTGTTCCAGATTTACAATAAAGATTTCGCCAAGGGCCATCACGGTAAGCCCA
ACCTGCATACTTTATATTGGACAGGTTTATTCAGCCCAGAAAATTTAGCGAAAACAA
GCATCAAACTGAACGGCCAGGCTGAACTGTTCTATCGCCCGAAAAGTCGCATGAAAC
GCATGGCACATCGTTTAGGTGAAAAGATGTTGAACAAAAAACTTAAAGATCAGAAAA
CTCCGATTCCTGACACTCTCTACCAAGAACTGTACGATTACGTCAACCATCGTCTGA
GCCATGATCTGAGTGATGAAGCACGCGCTCTGCTGCCGAACGTGATTACCAAGGAAG
TCAGTCACGAAATTATTAAAGACCGCCGCTTCACATCCGATAAATTCTTCTTCCACG
TGCCAATTACGCTTAATTACCAGGCAGCAAACTCTCCTTCCAAATTTAATCAGCGCG
TTAATGCGTATCTGAAAGAGCACCCGGAAACACCGATCATTGGTATCGCCCGCGGGG
AACGTAATCTCATCTATATTACTGTTATTGATTCGACCGGCAAAATTCTGGAGCAGC
GCTCTCTTAATACGATCCAGCAGTTTGACTATCAAAAAAAGCTGGACAACCGCGAGA
AGGAACGCGTCGCGGCCCGCCAAGCCTGGAGCGTTGTTGGCACAATCAAAGATCTGA
AGCAGGGTTACCTTAGTCAAGTTATCCACGAAATTGTTGATCTTATGATTCATTATC
AGGCCGTTGTGGTGCTGGAAAACCTTAACTTTGGCTTTAAAAGCAAACGCACGGGGA
TCGCTGAAAAAGCGGTATACCAGCAGTTCGAAAAAATGCTGATTGATAAACTCAATT
GCCTCGTGCTGAAAGATTACCCGGCTGAAAAAGTGGGTGGTGTGCTGAATCCATATC
AACTGACAGATCAATTCACCTCTTTTGCTAAAATGGGCACTCAAAGCGGTTTTTTGT
TTTACGTCCCGGCGCCCTATACTAGCAAGATCGACCCGCTGACAGGTTTTGTGGATC
CGTTCGTTTGGAAAACCATCAAAAATCATGAATCGCGTAAACACTTTCTGGAGGGCT
TTGATTTCTTGCACTATGATGTGAAAACCGGCGATTTTATCCTGCATTTTAAGATGA
ACCGTAATTTGAGCTTCCAGCGCGGTCTGCCAGGTTTCATGCCCGCGTGGGACATTG
TTTTCGAAAAGAATGAGACACAGTTCGACGCCAAAGGAACCCCGTTTATCGCGGGTA
AACGCATTGTTCCGGTCATCGAGAATCATCGTTTTACCGGTCGTTACCGTGATTTGT
ATCCTGCAAACGAACTCATCGCATTGTTGGAAGAAAAGGGAATCGTGTTTCGTGATG
GTTCGAATATCCTGCCTAAACTGCTGGAGAACGATGACTCACACGCTATTGATACCA
TGGTCGCTCTGATCCGCTCGGTCTTACAGATGCGTAACAGCAACGCGGCTACGGGCG
AGGATTACATTAACTCGCCGGTTCGCGATCTGAACGGCGTCTGCTTTGACTCGCGCT
TTCAAAATCCAGAATGGCCGATGGATGCGGACGCGAATGGTGCGTACCACATCGCTC
TTAAAGGGCAGTTACTGTTAAACCATTTGAAAGAATCTAAAGACCTGAAGCTGCAAA
ACGGTATCAGCAACCAGGATTGGCTCGCGTATATCCAAGAACTGCGTAAC
PAM Position: 5′
PAM: TTTN
Guide RNA Scaffold:
Nucleotide Sequence
(SEQ ID NO: 24)
TAATTTCTACTCTTGTAGAT
Reference: Zetsche, et al. (2015) Cell 163.3:759-71
13. Variant Name: dLbCpf1
Variant DNA:
Nucleotide Sequence
(SEQ ID NO: 25)
ATGAGCAAGCTGGAGAAGTTTACAAACTGCTACTCCCTGTCTAAGACCCTGA
GGTTCAAGGCCATCCCTGTGGGCAAGACCCAGGAGAACATCGACAATAAGCGGCTGC
TGGTGGAGGACGAGAAGAGAGCCGAGGATTATAAGGGCGTGAAGAAGCTGCTGGATC
GCTACTATCTGTCTTTTATCAACGACGTGCTGCACAGCATCAAGCTGAAGAATCTGA
ACAATTACATCAGCCTGTTCCGGAAGAAAACCAGAACCGAGAAGGAGAATAAGGAGC
TGGAGAACCTGGAGATCAATCTGCGGAAGGAGATCGCCAAGGCCTTCAAGGGCAACG
AGGGCTACAAGTCCCTGTTTAAGAAGGATATCATCGAGACAATCCTGCCAGAGTTCC
TGGACGATAAGGACGAGATCGCCCTGGTGAACAGCTTCAATGGCTTTACCACAGCCT
TCACCGGCTTCTTTGATAACAGAGAGAATATGTTTTCCGAGGAGGCCAAGAGCACAT
CCATCGCCTTCAGGTGTATCAACGAGAATCTGACCCGCTACATCTCTAATATGGACA
TCTTCGAGAAGGTGGACGCCATCTTTGATAAGCACGAGGTGCAGGAGATCAAGGAGA
AGATCCTGAACAGCGACTATGATGTGGAGGATTTCTTTGAGGGCGAGTTCTTTAACT
TTGTGCTGACACAGGAGGGCATCGACGTGTATAACGCCATCATCGGCGGCTTCGTGA
CCGAGAGCGGCGAGAAGATCAAGGGCCTGAACGAGTACATCAACCTGTATAATCAGA
AAACCAAGCAGAAGCTGCCTAAGTTTAAGCCACTGTATAAGCAGGTGCTGAGCGATC
GGGAGTCTCTGAGCTTCTACGGCGAGGGCTATACATCCGATGAGGAGGTGCTGGAGG
TGTTTAGAAACACCCTGAACAAGAACAGCGAGATCTTCAGCTCCATCAAGAAGCTGG
AGAAGCTGTTCAAGAATTTTGACGAGTACTCTAGCGCCGGCATCTTTGTGAAGAACG
GCCCCGCCATCAGCACAATCTCCAAGGATATCTTCGGCGAGTGGAACGTGATCCGGG
ACAAGTGGAATGCCGAGTATGACGATATCCACCTGAAGAAGAAGGCCGTGGTGACCG
AGAAGTACGAGGACGATCGGAGAAAGTCCTTCAAGAAGATCGGCTCCTTTTCTCTGG
AGCAGCTGCAGGAGTACGCCGACGCCGATCTGTCTGTGGTGGAGAAGCTGAAGGAGA
TCATCATCCAGAAGGTGGATGAGATCTACAAGGTGTATGGCTCCTCTGAGAAGCTGT
TCGACGCCGATTTTGTGCTGGAGAAGAGCCTGAAGAAGAACGACGCCGTGGTGGCCA
TCATGAAGGACCTGCTGGATTCTGTGAAGAGCTTCGAGAATTACATCAAGGCCTTCT
TTGGCGAGGGCAAGGAGACAAACAGGGACGAGTCCTTCTATGGCGATTTTGTGCTGG
CCTACGACATCCTGCTGAAGGTGGACCACATCTACGATGCCATCCGCAATTATGTGA
CCCAGAAGCCCTACTCTAAGGATAAGTTCAAGCTGTATTTTCAGAACCCTCAGTTCA
TGGGCGGCTGGGACAAGGATAAGGAGACAGACTATCGGGCCACCATCCTGAGATACG
GCTCCAAGTACTATCTGGCCATCATGGATAAGAAGTACGCCAAGTGCCTGCAGAAGA
TCGACAAGGACGATGTGAACGGCAATTACGAGAAGATCAACTATAAGCTGCTGCCCG
GCCCTAATAAGATGCTGCCAAAGGTGTTCTTTTCTAAGAAGTGGATGGCCTACTATA
ACCCCAGCGAGGACATCCAGAAGATCTACAAGAATGGCACATTCAAGAAGGGCGATA
TGTTTAACCTGAATGACTGTCACAAGCTGATCGACTTCTTTAAGGATAGCATCTCCC
GGTATCCAAAGTGGTCCAATGCCTACGATTTCAACTTTTCTGAGACAGAGAAGTATA
AGGACATCGCCGGCTTTTACAGAGAGGTGGAGGAGCAGGGCTATAAGGTGAGCTTCG
AGTCTGCCAGCAAGAAGGAGGTGGATAAGCTGGTGGAGGAGGGCAAGCTGTATATGT
TCCAGATCTATAACAAGGACTTTTCCGATAAGTCTCACGGCACACCCAATCTGCACA
CCATGTACTTCAAGCTGCTGTTTGACGAGAACAATCACGGACAGATCAGGCTGAGCG
GAGGAGCAGAGCTGTTCATGAGGCGCGCCTCCCTGAAGAAGGAGGAGCTGGTGGTGC
ACCCAGCCAACTCCCCTATCGCCAACAAGAATCCAGATAATCCCAAGAAAACCACAA
CCCTGTCCTACGACGTGTATAAGGATAAGAGGTTTTCTGAGGACCAGTACGAGCTGC
ACATCCCAATCGCCATCAATAAGTGCCCCAAGAACATCTTCAAGATCAATACAGAGG
TGCGCGTGCTGCTGAAGCACGACGATAACCCCTATGTGATCGGCATCGCTAGGGGCG
AGCGCAATCTGCTGTATATCGTGGTGGTGGACGGCAAGGGCAACATCGTGGAGCAGT
ATTCCCTGAACGAGATCATCAACAACTTCAACGGCATCAGGATCAAGACAGATTACC
ACTCTCTGCTGGACAAGAAGGAGAAGGAGAGGTTCGAGGCCCGCCAGAACTGGACCT
CCATCGAGAATATCAAGGAGCTGAAGGCCGGCTATATCTCTCAGGTGGTGCACAAGA
TCTGCGAGCTGGTGGAGAAGTACGATGCCGTGATCGCCCTGGAGGACCTGAACTCTG
GCTTTAAGAATAGCCGCGTGAAGGTGGAGAAGCAGGTGTATCAGAAGTTCGAGAAGA
TGCTGATCGATAAGCTGAACTACATGGTGGACAAGAAGTCTAATCCTTGTGCAACAG
GCGGCGCCCTGAAGGGCTATCAGATCACCAATAAGTTCGAGAGCTTTAAGTCCATGT
CTACCCAGAACGGCTTCATCTTTTACATCCCTGCCTGGCTGACATCCAAGATCGATC
CATCTACCGGCTTTGTGAACCTGCTGAAAACCAAGTATACCAGCATCGCCGATTCCA
AGAAGTTCATCAGCTCCTTTGACAGGATCATGTACGTGCCCGAGGAGGATCTGTTCG
AGTTTGCCCTGGACTATAAGAACTTCTCTCGCACAGACGCCGATTACATCAAGAAGT
GGAAGCTGTACTCCTACGGCAACCGGATCAGAATCTTCCGGAATCCTAAGAAGAACA
ACGTGTTCGACTGGGAGGAGGTGTGCCTGACCAGCGCCTATAAGGAGCTGTTCAACA
AGTACGGCATCAATTATCAGCAGGGCGATATCAGAGCCCTGCTGTGCGAGCAGTCCG
ACAAGGCCTTCTACTCTAGCTTTATGGCCCTGATGAGCCTGATGCTGCAGATGCGGA
ACAGCATCACAGGCCGCACCGACGTGGATTTTCTGATCAGCCCTGTGAAGAACTCCG
ACGGCATCTTCTACGATAGCCGGAACTATGAGGCCCAGGAGAATGCCATCCTGCCAA
AGAACGCCGACGCCAATGGCGCCTATAACATCGCCAGAAAGGTGCTGTGGGCCATCG
GCCAGTTCAAGAAGGCCGAGGACGAGAAGCTGGATAAGGTGAAGATCGCCATCTCTA
ACAAGGAGTGGCTGGAGTACGCCCAGACCAGCGTGAAGCAC
PAM Position: 5′
PAM: TTTN
Guide RNA Scaffold:
Nucleotide Sequence
(SEQ ID NO: 26)
TAATTTCTACTAAGTGTAGAT
Reference: Zetsche, et al. (2015) Cell 163.3:759-71

All publications, patents and sequence database entries mentioned in the specification herein are hereby incorporated by reference in their entirety as if each individual publication or patent was specifically and individually indicated to be incorporated by reference. In case of conflict, the present application, including any definitions herein, will control.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

What is claimed is:

1. A method of controlling expression of a first output sequence, comprising:
introducing into a cell a genetic circuit comprising one or more polynucleotide sequences, wherein the genetic circuit comprises:
(a) a first output sequence;
(b) a first promoter/operator controlling transcription of the first output sequence;
(c) a first guide RNA targeting the first promoter/operator;
(d) a second promoter/operator controlling transcription of the first guide RNA, wherein the second promoter/operator is input-sensitive such that a first input signal is required for induction of transcription of the first guide RNA;
(e) a first catalytically inactive endonuclease that in combination with the first guide RNA binds to a sequence targeted by the first guide RNA and prevents transcription of the first output sequence;
(f) a third promoter/operator controlling transcription of the first catalytically inactive endonuclease, wherein the third promoter/operator is input-sensitive such that a second input signal is required for induction of transcription of the first catalytically inactive endonuclease; and
(g) one or more heterologous polymerases that specifically bind one or more of the first, second and/or third promoter/operator.

2. The method of claim 1, wherein the genetic circuit further comprises a fourth promoter/operator controlling transcription of one or more second output sequence, wherein the fourth promoter/operator is input-sensitive such that a third input signal is required for induction of transcription of the one or more second output sequence.

3. The method of claim 1 or claim 2, wherein the genetic circuit further comprises one or more endogenous polymerases that bind one or more of the first, second, third and/or fourth promoter/operator.

4. The method of claim 2 or claim 3, wherein the third input signal is one or more second guide RNA encoded by the first output sequence and wherein the one or more second output sequence is one or more third guide RNA.

5. The method of any one of claims 1-4, wherein at least two of the first, second and third input signals are the same.

6. The method of any one of claims 1-5, wherein any of the first, second, third and/or fourth promoter/operator comprises a T7 promoter and an operator.

7. The method of any one of claims 1-6, wherein the heterologous polymerase is a viral polymerase.

8. The method of any one of claims 1-7, wherein the heterologous polymerase is a T7 RNA polymerase.

9. The method of any one of claims 1-8, wherein the genetic circuit further comprises polynucleotide sequences encoding one or more decoy operators having the same, or substantially the same, sequence as one or more of the operator sequences of the first, second, third and/or fourth promoter/operators.

10. The method of any one of claims 1-9, wherein the genetic circuit further comprises one or more polynucleotide sequences encoding one or more small RNAs (sRNAs) that binds to and sequesters the guide RNA.

11. The method of any one of claims 1-10, wherein the first, second and/or third guide RNA is a nested guide RNA comprising two or more sequences that target two or more target sequences in the first, second, third and/or fourth promoter/operator, thereby causing promoter looping of the first, second, third and/or fourth promoter/operator.

12. The method of any one of claims 1-11, wherein the first, second and/or third input signal is a chemical, light, a polypeptide or a mechanical force.

13. The method of any one of claims 1-12, wherein the first, second and/or third input signal is isopropyl β-D-1-thiogalactopyranoside (IPTG), anhydrotetracycline (aTc), or 2,4-diacetylphloroglucinol (DAPG).

14. The method of any one of claims 1-13, wherein the first and/or second output sequence encode one or more first output molecule and the one or more first output molecule in turn becomes a fourth input signal required for controlling transcription of one or more third output sequence.

15. The method of claim 14, wherein the first second and/or third output sequence is a fourth guide RNA.

16. The method of any one of claims 1-15, wherein two or more input signals control transcription in a single promoter/operator.

17. The method of any one of claims 1-16, wherein the first catalytically inactive endonuclease is a RNA-guided DNA endonuclease.

18. The method of any one of claims 1-17, wherein the first catalytically inactive endonuclease is a catalytically inactive clustered regularly interspaced short palindromic repeat (CRISPR) endonuclease.

19. The method of any one of claims 1-18, wherein the first catalytically inactive endonuclease is catalytically inactive Cas9 or catalytically inactive Cpf1.

20. The method of any one of claims 1-19, wherein the first catalytically inactive endonuclease is selected from the group consisting of dSpCas9, dSpCas9(E), dSpCas9(VRER), dSpCas9(VQR), dSpCas9(EQR), desSpCas9, dSpCas9-HF1, dSaCas9, desSaCas9, dSt1Cas9, dFnCpf1, dAsCpf1, and dLbCpf1.

21. The method of any one of claims 1-20, wherein one or more nucleotide of the first, second, third and/or fourth guide RNA is mutated and the mutation of the first, second, third and/or fourth guide RNA does not decrease prevention of transcription of the first, second and/or third output sequence.

22. The method of any one of claims 1-21, wherein the cell is a prokaryotic cell.

23. The method of any one of claims 1-22, wherein the cell is a bacterial cell.

24. The method of claim 23, wherein the genetic circuit in the bacterial cell is optimized for bioreactor growth.

25. The method of any one of claims 1-24, wherein the cell is part of a microbiome.

26. The method of any one of claims 1-22, wherein the cell is a BL21(DE3) cell.

27. The method of any one of claims 1-21, wherein the cell is a eukaryotic cell.

28. The method of any one of claims 1-21, wherein the cell is a plant cell.

29. The method of any one of claims 1-21, wherein the cell is a human cell.

30. The method of any one of claims 1-29, wherein the first, second, third and/or fourth promoter/operator is sensitive to a guide RNA.

31. The method of any one of claims 1-29, wherein the first, second, third and/or fourth promoter/operator is sensitive to a chemical input.

32. The method of any one of claims 1-31, wherein the first, second, third and/or fourth promoter/operator comprises a polynucleotide sequence that encodes a T7 promoter and a polynucleotide sequence that encodes a PhlF operator.

33. The method of claim 32, wherein the T7 promoter and the PhlF operator control the transcription of guide RNA A2NT.

34. The method of claim 33, wherein the guide RNA A2NT controls the transcription of the A2NT operator.

35. The method of any one of claims 1-34, wherein the first, second and/or third output sequence is a DNA sequence.

36. The method of any one of claims 33-35, wherein the first, second and/or third output sequence encodes one or more second output molecule.

37. The method of claim 36, wherein the first and/or second output molecule controls a fifth heterologous promoter/operator controlling transcription of one or more fourth output sequence.

38. The method of claim 36 or claim 37, wherein the first and/or second output molecule is a protein.

39. The method of any one of claims 1-35, further comprising culturing the cell under conditions that allow expression of the genetic circuit in the cell.

40. A genetic circuit, comprising:
(a) a first output sequence;
(b) a first promoter/operator controlling transcription of the first output sequence;
(c) a first guide RNA targeting the first promoter/operator;
(d) a second promoter/operator controlling transcription of the first guide RNA, wherein the second promoter/operator is input-sensitive such that a first input signal is required for induction of transcription of the first guide RNA;
(e) a first catalytically inactive endonuclease that in combination with the first guide RNA binds to a sequence targeted by the first guide RNA and prevents transcription of the first output sequence;
(f) a third promoter/operator controlling transcription of the first catalytically inactive endonuclease, wherein the third promoter/operator is input-sensitive such that a second input signal is required for induction of transcription of the first catalytically inactive endonuclease; and
(g) one or more heterologous polymerases that specifically bind one or more of the first, second and/or third promoter/operator.

41. The genetic circuit of claim 40, wherein the genetic circuit further comprises a fourth promoter/operator controlling transcription of one or more second output sequence, wherein the fourth promoter/operator is input-sensitive such that a third input signal is required for induction of transcription of the one or more second output sequence.

42. The genetic circuit of claim 40 or claim 41, wherein the genetic circuit further comprises one or more endogenous polymerases that bind one or more of the first, second, third and/or fourth promoter/operator.

43. The genetic circuit of claim 41 or claim 42, wherein the third input signal is one or more second guide RNA encoded by the first output sequence and wherein the one or more second output sequence is one or more third guide RNA.

44. The genetic circuit of any one of claims 40-43, wherein at least two of the first, second and third input signals are the same.

45. The genetic circuit of any one of claims 40-44, wherein any of the first, second, third and/or fourth promoter/operators comprises a T7 promoter and an operator.

46. The genetic circuit of any one of claims 40-45, wherein the heterologous polymerase is a viral polymerase.

47. The genetic circuit of any one of claims 40-46, wherein the heterologous polymerase is a T7 RNA polymerase.

48. The genetic circuit of any one of claims 40-47, wherein the genetic circuit further comprises polynucleotide sequences encoding one or more decoy operators having the same, or substantially the same, sequence as one or more of the operator sequences of the first, second, third and/or fourth promoter/operators.

49. The genetic circuit of any one of claims 40-48, wherein the genetic circuit further comprises one or more polynucleotide sequences encoding one or more small RNAs (sRNAs) that binds to and sequesters the guide RNA.

50. The genetic circuit of any one of claims 40-49, wherein the first, second, and/or third guide RNA is a nested guide RNA comprising two or more sequences that target two or more target sequences in the first, second, third and/or fourth promoter/operator, thereby causing promoter looping of the first, second, third and/or fourth promoter/operator.

51. The genetic circuit of any one of claims 40-50, wherein the first, second and/or third input signal is a chemical, light, a polypeptide or a mechanical force.

52. The genetic circuit of any one of claims 40-51, wherein the first, second and/or third input signal is isopropyl β-D-1-thiogalactopyranoside (IPTG), anhydrotetracycline (aTc), or 2,4-diacetylphloroglucinol (DAPG).

53. The genetic circuit of any one of claims 40-52, wherein the first and/or second output sequence encode one or more first output molecule and the one or more first output molecule in turn becomes a fourth input signal required for controlling transcription of one or more third output sequences.

54. The method of claim 53, wherein the first, second and/or third output sequence is a fourth guide RNA.

55. The genetic circuit of any one of claims 40-53, wherein two or more input signals control transcription in a single promoter/operator.

56. The genetic circuit of any one of claims 40-55, wherein the first catalytically inactive endonuclease is a RNA-guided DNA endonuclease.

57. The genetic circuit of any one of claims 40-56, wherein the first catalytically inactive endonuclease is a catalytically inactive clustered regularly interspaced short palindromic repeat (CRISPR) endonuclease.

58. The genetic circuit of any one of claims 40-57, wherein the first catalytically inactive endonuclease is catalytically inactive Cas9 or catalytically inactive Cpf1.

59. The genetic circuit of any one of claims 40-58, wherein the first catalytically inactive endonuclease is selected from the group consisting of dSpCas9, dSpCas9(E), dSpCas9(VRER), dSpCas9(VQR), dSpCas9(EQR), desSpCas9, dSpCas9-HF1, dSaCas9, desSaCas9, dSt1Cas9, dFnCpf1, dAsCpf1, and dLbCpf1.

60. The genetic circuit of any one of claims 40-59, wherein one or more nucleotide of the first, second, third and/or fourth guide RNA is mutated and the mutation of the first, second, third and/or fourth guide RNA does not decrease prevention of transcription of the first, second and/or third output sequence.

61. The genetic circuit of any one of claims 40-60, wherein the first, second, third and/or fourth promoter/operator is sensitive to a guide RNA.

62. The genetic circuit of any one of claims 40-60, wherein the first, second, third and/or fourth promoter/operator is sensitive to a chemical input.

63. The genetic circuit of any one of claims 40-62, wherein the first, second, third and/or fourth promoter/operator comprises a polynucleotide sequence that encodes a T7 promoter and a polynucleotide sequence that encodes a PhlF operator.

64. The genetic circuit of claim 63, wherein the T7 promoter and the PhIF operator control the transcription of guide RNA A2NT.

65. The genetic circuit of claim 64, wherein the guide RNA A2NT controls the transcription of the A2NT operator.

66. The genetic circuit of any one of claims 40-65, wherein one or more of the first, second and/or third output sequence is a DNA sequence.

67. The genetic circuit of any one of claims 40-66, wherein the first, second and/or third output sequence encodes a second output molecule.

68. The genetic circuit of claim 67, wherein the first and/or second output molecule controls a fifth heterologous promoter/operator controlling transcription of one or more fourth output sequences.

69. The genetic circuit of claim 67 or claim 68, wherein the first and/or second output molecule is a protein.
